(19) United States
(12) Patent Application Publication
(10) Pub. No.: US 2009/0137662 A1
(43) Pub. Date: May 28, 2009

(54) SYNERGY OF DOPAMINE D2 AND ADENOSINE A2 RECEPTORS ACTIVATES PKA SIGNALING VIA BETA/GAMMA DIMERS

(75) Inventors: Adrienne S. Gordon, Emeryville, CA (US); Ivan F. Diamond, Palo Alto, CA (US); Lina Yao, Palo Alto, CA (US)

Correspondence Address: JONES DAY, 222 EAST 41ST ST, NEW YORK, NY, 10017, US

(73) Assignee: The Regents of the University of California

(21) Appl. No.: 10/550,331

(22) PCT Filed: Mar. 27, 2003
(86) PCT No.: PCT/US2003/009629

§ 371 Date: Feb. 22, 2007

Related U.S. Application Data
(60) Provisional application No. 60/368,417, filed on Mar. 27, 2002.

(51) Int. Cl. A61K 31/352; C12Q 1/02; C12Q 1/68

(52) U.S. Cl. 514/455; 435/29; 435/6

(57) ABSTRACT

This invention pertains to the discovery that a dopamine receptor agonist can activate PKA signaling and/or can act synergistically with an adenosine receptor to activate such signaling. In various embodiments, this invention exploits the synergy between the dopamine receptor pathway and an adenosine receptor pathway to provide methods of mitigating one or more symptoms produced by the chronic consumption of a substance of abuse or to mitigate one or more physiological and/or behavioral symptoms associated with cessation of chronic consumption of a substance of abuse. In certain embodiments, the methods involve administering to the mammal an effective amount of an adenosine receptor antagonist; and an effective amount of a dopamine receptor antagonist; where the effective amount of the adenosine receptor antagonist is lower than the effective amount of an adenosine receptor antagonist administered without said dopamine receptor antagonist.

### CROSS-REFERENCE TO RELATED APPLICATIONS

This application claims priority to and benefit of U.S. Provisional Application No. 60/368,417, filed on Mar. 27, 2002, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was supported in part by Grant Nos. NIH AA10030 and NTH AA10039 from the National Institutes of Health. The Government of the United States of America may have certain rights in this invention.

### FIELD OF THE INVENTION

The present invention is in the field of neurobiology. In particular, this invention provides methods of mitigating one or more symptoms associated with chronic consumption of substances of abuse and methods of screening for agents that mitigate such symptoms.

### BACKGROUND OF THE INVENTION

The abuse of ethanol and other substances of abuse remains a major public health problem in the U.S. and throughout the world. Chronic alcoholism causes functional and pathologic changes in many organs, particularly the brain, but the molecular mechanisms which account for these effects are not well understood.

It has been shown that ethanol-induced changes in cAMP signal transduction appear to play a role in the acute and chronic effects of ethanol. See, for example, Nagy et al. (1988) Proc. Natl. Acad. Sci. USA, 85: 6973-6976; Gordon et al. (1986) Proc. Natl. Acad. Sci., USA, 83: 2105-2108; Valverius et al. (1987) Mol. Pharmac. 32:217-227; and Charness et al. (1988) Biochem. Biophys. Res. Comm. 155: 138-143. Short term ethanol exposure increases receptor-stimulated cAMP levels in NG108-15 neuroblastoma×glioma hybrids (Gordon et al. (1986) supra.). In contrast, chronic exposure to ethanol causes a decrease in receptor-dependent cAMP levels (Gordon et al. (1986) supra, and Charness et al. (1988) supra.). This reduction appears to be significant in chronic alcoholism since cells from alcoholics exhibit decreases in both adenosine receptor-stimulated and PGE₁ receptor-stimulated cAMP levels (Nagy et al. (1988) supra.; and Diamond et al. (1987) Proc. Natl. Acad. Sci., USA, 84: 1413-1416.

It is also of interest to provide assays useful for the evaluation of drugs that may be used to treat one or more of the adverse effects of chronic over-consumption of ethanol or other addictive drugs. It is also of interest to provide methods for the detection of exposure in individuals to ethanol or other substances of abuse so as to monitor compliance with abuse treatment regimens.

### SUMMARY OF THE INVENTION

This invention pertains to the elucidation of metabolic pathways underlying the cellular response to consumption or chronic consumption of a substance of abuse (e.g. ethanol, opiates, barbiturates, cannabinoids, etc.). In particular, this invention pertains to the discovery that a dopamine receptor (D2) pathway is coupled to an adenosine receptor pathway (a PKA pathway). In particular, it is demonstrated herein that a D2 agonist can activate PKA signaling and/or can act synergistically with an adenosine receptor to activate such signaling.

In various embodiments, this invention exploits the synergy between the a dopamine receptor pathway and an adenosine receptor pathway to provide methods of mitigating one or more symptoms produced by the chronic consumption of a substance of abuse or to mitigate one or more physiological and/or behavioral symptoms associated with cessation of consumption (e.g. chronic consumption) of a substance of abuse. Thus, in certain embodiments, this invention provides a method of mitigating one or more symptoms associated with chronic consumption of a substance of abuse by a mammal. The methods involve administering to the mammal an effective amount of an adenosine receptor antagonist; and an effective amount of a dopamine receptor antagonist; where the effective amount of the adenosine receptor antagonist is lower than the effective amount of an adenosine receptor antagonist administered without said dopamine receptor antagonist.

In certain other embodiments, this invention provides methods of mitigating one or more symptoms associated with cessation of consumption (e.g. chronic consumption) of a substance of abuse by a mammal. These methods typically involve comprising:

administering to the mammal an effective amount of an adenosine receptor agonist; and an effective amount of a dopamine receptor agonist; where the effective amount of the adenosine receptor agonist is lower than the effective amount of an adenosine receptor agonist administered without said dopamine receptor agonist.

Also provided are compositions comprising an adenosine receptor antagonist and a dopamine receptor antagonist or an adenosine receptor agonist and a dopamine receptor agonist.

In certain embodiments, the invention relates, in part, to the discovery that exposure to ethanol alters dramatically the subcellular localization of the catalytic Cα subunit and the RIα and RIIβ regulatory subunits of the cAMP dependent protein kinase (PKA) and the δ- and ε-isozymes of protein kinase C (PKC). Exposure to other addictive drugs alters the subcellular localization of the catalytic Cα subunit of PKA, δ-PKC and ε-PKC and is likely to alter the subcellular localization of other proteins as well. For example, the catalytic Cα subunit of PKA, which is normally localized to the Golgi apparatus area, appears to translocate to the nucleus upon exposure of a cell to ethanol. Ethanol also has been shown to cause translocation of PKC activity from cytosolic to membrane fraction in astroglial cells and human lymphocytes and epidermal keratinocytes. The present invention further relates to the discovery that the detectable amount of the regulatory subunit RI of PKA decreases, and the amounts of the α-, δ- and ε-subunits of PKC increase in certain cell types, including but not limited to, NG108-15 cells (α-, δ-, and ε-subunit) or PC12 cells (δ- and ε-subunit), upon the exposure to ethanol. These discoveries provide the basis for assays that may be used to detect the exposure of cells to ethanol or other addictive drugs and further for assays that may be used for the screening of drugs or treatment to modulate the effects of consumption of ethanol or other addictive drugs.

One aspect of the invention is to provide assays that provide an indication of the exposure of a cell or an individual to an addictive drug by identifying at least one cell component, e.g., a protein, that has a cellular localization (distribution) that varies in correlation with the exposure of the cell to the addictive drug, and determining the distribution of that cell component within a cell of a sample to be tested. In one preferred embodiment, the cell component comprises a subunit of the cAMP dependent protein kinase, PKA, the Cα subunit being particularly preferred. In another preferred embodiment, the cellular component comprises an isozyme of protein kinase C, PKC, wherein the δ or the ε isozyme of protein kinase C is particularly preferred. The invention also provides assays for determining chronic exposure to an addictive drug and methods for determining whether a mammal has been chronically consuming an addictive drug.

Another aspect of the invention is to provide assays that provide an indication of exposure of a cell or an individual to an addictive drug by measuring the amount of protein that varies in amount in a relationship with the exposure of the cell to the addictive drug. In one preferred embodiment, the decrease of the detectable amount of the regulatory subunit RI of PKA in response to exposure is determined. In another preferred embodiment, the increase of the detectable amounts of α-PKC, δ-PKC, or ε-PKC in response to exposure is measured.

Another aspect of the invention is to provide assays for screening therapeutic compounds that modulate or mimic the effects of an addictive drug on a cell. Some of these screening assays measure the ability of a compound of interest to interfere with one or more of the cellular effects of an addictive drug described herein, e.g., changes in the localization of PKA Cα, PKA RIα, PKA RIIβ, δ-PKC, or ε-PKC, decreases in the amount of RI, increases in the amount of αPKC, δ-PKC, or ε-PKC, changes in the set of proteins phosphorylated by or expressed in response to Cα, δ-PKC and ε-PKC. For example, Cα may induce phosphorylation of (cAMP response element binding protein) CREB (cAMP response element binding protein) and thereby its activation, resulting in the induction of gene expression regulated by the cyclic AMP response element (CRE).

Another aspect of the invention is to provide kits for detecting the exposure of cells to an addictive drug or for identifying a substance that alters or mimics the effects of an addictive drug. Kits of the invention may include labeled antibodies capable of specifically binding to Cα, RIα and RIIβ subunits of PKA or to the α-, δ- or ε-isozymes of PKC.

Yet another aspect of the invention is to provide methods for reducing consumption of an addictive drug comprising administering a substance which alters or mimics the effects of the addictive drug on the cellular localization of, e.g., PKA Cα, PKA RIα, PKA RIIβ, δ-PKC, or ε-PKC.

Another aspect of the invention is to provide methods for reducing consumption of an addictive drug comprising administering two substances that modulate a receptor for the addictive drug and a factor that acts synergistically with the receptor in the alteration of the sub cellular of these proteins.

### DEFINITIONS

The term “substance of abuse” refers to a substance that is psychoactive and that induces tolerance and/or addiction. Substances of abuse include, but are not limited to stimulants (e.g. cocaine, amphetamines), opiates (e.g. morphine, heroin), cannabinoids (e.g. marijuana, hashish), nicotine, alcohol, substances that mediate agonist activity at the dopamine D2 receptor, and the like. Substances of abuse include, but are not limited to addictive drugs.

A “dopamine receptor antagonist” refers to a substance that reduces or blocks activity mediated by a dopamine receptor in response to the cognate ligand of that receptor. Thus, for example, a dopamine receptor antagonist will reduce or eliminate the activity of dopamine mediated by a dopamine receptor and associated pathway(s). The activity of the antagonist can be directly at the receptor, e.g., by blocking the receptor or by altering receptor configuration or activity of the receptor. The activity of the antagonist can also be at other points (e.g. at one or more second messengers, kinases, etc.) in a metabolic pathway that mediates the receptor activity.

An “adenosine receptor antagonist” refers to a substance refers to a substance that reduces or blocks activity mediated by an adenosine receptor in response to the cognate ligand of that receptor. Thus, for example, an adenosine receptor antagonist will reduce or eliminate the activity of adenosine mediated by an adenosine receptor and associated pathway(s). The activity of the antagonist can be directly at the receptor, e.g., by blocking the receptor or by altering receptor configuration or activity of the receptor. The activity of the antagonist can also be at other points (e.g. at one or more second messengers, kinases, etc.) in a metabolic pathway that mediates the receptor activity. Two distinct adenosine receptor classes exist, designated A₁ receptors (including subclasses which inhibit adenylate cyclase activity when activated) and A₂ receptors that stimulate adenylate cyclase activity when activated. In certain embodiments, adenosine antagonists of this invention are capable of combining with either or both of these receptors but are incapable of stimulating the normal activity that occurs when adenosine binds to such receptors. As a result, stimulation upon subsequent exposure to adenosine or adenosine agonists will be blocked or inhibited.

As used herein, the term “adenosine receptor agonist” means an agent capable of combining with A₁ and/or the A₂ receptor and capable of stimulating the associated receptor activity. The term adenosine agonist will also include partial adenosine agonists that are capable of partially stimulating adenosine receptor activity, i.e., providing a lesser activity than would be obtained with a like concentration of adenosine.

As used herein, the term “dopamine receptor agonist” means an agent capable of combining with D2 dopamine receptor and capable of stimulating the associated receptor activity. The term dopamine receptor agonist will also include partial dopamine receptor agonists that are capable of partially stimulating D2 activity, i.e., providing a lesser activity than would be obtained with a like concentration of dopamine.

An “ethanol indicative protein” is a protein whose subcellular location or detectable amount is different in a cell exposed to ethanol compared to a cell that is not exposed to ethanol. Similarly, a “drug indicative protein” is a protein whose subcellular location or detectable amount is different in a cell exposed to an addictive drug compared to a cell that is not exposed to such addictive drug.

An “ethanol-exposed cell” (“EEC”) is a cell which is or has been contacted with exogenous ethanol. An “ethanol-unexposed cell” (“EUC”) is a cell which has not been contacted with exogenous ethanol. By “exogenous ethanol” is meant ethanol which has been introduced from outside a cell or organism. For example, exogenous ethanol can be ethanol added to a sample (e.g., a cell culture) or ethanol consumed by a mammal from which a sample is obtained. Similarly, a “drug-exposed cell” (“DEC”) is a cell which is or has been contacted with an exogenous addictive drug, and a “drug-unexposed cell” (“DUC”) is a cell which has not been contacted with an exogenous addictive drug.

“Cellular localization” of an ethanol indicative protein refers to a cellular subregion in which the protein is present. A protein is “present” in a cellular subregion if it can be detected in the cellular subregion by any of the techniques known in the art and/or discussed herein.

A “cellular subregion” is a region within the cell that is detectably distinct from another region of the cell. Examples of cellular subregions include, but are not limited to, the nucleus, the perinucleus, the Golgi apparatus, and the cytoplasm. The “cytoplasm” is the region of the cell outside the perinucleus and can include structures present in the cytosol, such as, for example, the endoplasmic reticulum, mitochondria, lysosomes, peroxisomes, vacuoles, other cytoplasmic organelles, and other structures such as cytoskeletal filaments, etc. In smaller cells, such as lymphocytes, the Golgi apparatus may not be distinguishable from the rest of the cytoplasm. However, in larger cells such as NG108 cells, the Golgi apparatus and some of the structures described above may be visually distinguishable from the rest of the cytoplasm.

An “origin cellular subregion” is a cellular subregion in which a given ethanol indicative protein is predominantly present in an EUC. A “destination cellular subregion” is a cellular subregion in which an ethanol indicative protein is predominantly present in an EEC. In certain cells, some proteins may translocate to an intermediate destination cellular subregion and complete translocation to the destination cellular subregion as the duration of exposure increases. A “first cellular subregion” is typically the cellular subregion first discussed in a paragraph or a claim of this application. Depending on the context of the discussion, a first cellular subregion may be an origin cellular subregion or a destination cellular subregion. The phrase may also describe a plurality of cellular subregions where a protein is localized in more than one cellular subregions in a test cell and one or more cellular subregions in a control cell, or vice versa. For example, δ-PKC is predominantly present in the Golgi apparatus of EUCs and in both the perinucleus and nucleus of EECs. Thus, the nucleus is a destination subregion for δ-PKC and the perinucleus is also a destination subregion for δ-PKC.

A protein is “predominantly present” in a cellular subregion if a detectably greater amount of the protein is present in that cellular subregion compared to another cellular subregion.

A “detectably different amount (or number)” refers to a difference in an amount or number that can be detected when comparing cellular subregions within a cell, or when comparing cells, such as comparing a test cell(s) and a control cell(s). A detectably different amount means that more can be detected in one cellular subregion vs. another or in one cell compared to another. One can quantitate the amounts if desired, but quantitation is not necessary to practice many methods of the invention. Preferably, the difference is a statistically significant difference, more preferably a difference greater than about 5%, even more preferably a difference greater than about 25%, with a difference of greater than about 35% up to 100% being most preferred.

A substance that “mimics the effects of an addictive drug” or “that mimics the effect of a substance of abuse” on cellular localization affects the cellular localization of a protein in a similar manner as does the addictive drug or substance of abuse. For example, if the amount of the protein in a particular cellular subregion is (1) detectably greater in a DEC than a DUC, then it is detectably greater in a cell exposed to the substance than in a DUC; or (2) detectably less in a DEC than a DUC, then it is detectably less in a cell exposed to the substance. The amount of protein in the cellular subregion of a DEC need not be the same as the amount of protein in the corresponding cellular subregion of a cell exposed to the substance. Preferably, the amount of protein in the cellular subregion of a cell exposed to the substance differs from that in a DUC by a statistically significant amount, more preferably by more than about 5% and even more preferably approaches the amount of protein present in the cellular subregion of a DEC. For example, as discussed above, PICA Cα is present in a detectably greater amount in the nucleus of an EEC than in the nucleus of an EUC. Likewise, PKA Cα would also be present in a detectably greater amount in the nucleus of a cell exposed to a substance that mimics the effects of ethanol than in the nucleus of an EUC.

By “prior exposure” to ethanol or other substances of abuse (e.g., addictive drugs) is meant that a sample has been exposed to exogenous ethanol, or other substances of abuse, before a particular point in time, such as, for example, before testing for such exposure. Usually the sample has been exposed at most two weeks before testing, preferably less than a week, even more preferably within 48 hours before testing. An example of prior exposure is found in a sample obtained from a mammal that has a detectable blood alcohol level. However, the exposure need not be continuous and it need not occur immediately before testing. For example, many alcoholics have blood alcohol levels close to or equal to zero in the morning. Thus, the phrase “prior exposure” includes chronic and/or episodic exposure.

By “chronic exposure” to ethanol or other substances of abuse (e.g. addictive drugs) is meant that a sample or an organism has been exposed to exogenous ethanol or other substances of abuse chronically before a particular point in time. The sample might not have been exposed immediately before testing is performed, or even within 48 hours before testing, but it has been exposed on a recurrent or prolonged basis for a time sufficient for the cellular effects of such exposure to be detectable whether or not the substance of abuse (e.g. addictive drug) is present in the sample at a detectable level. An example of chronic exposure is found in a sample obtained from a mammal that has been chronically consuming alcohol whether or not the mammal has a detectable blood alcohol level at the time the sample is obtained.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “inhibit expression” is used with reference to inhibition of a beta/gamma dimer to refer to a reduction or blocking of beta/gamma dimer transcription, and/or translation, and/or formation or availability of active beta/gamma dimer.

The term a “beta/gamma dimmer nucleic acid” refers to a nucleic acid that encodes a polypeptide or a fragment of a polypeptide that comprises a beta gamma dimmer. Such a nucleic acid includes, but is not limited to genomic DNA, mRNA, cDNA, cRNA, or fragments thereof.

The term “specifically binds”, as used herein, when referring to a biomolecule (e.g., protein, nucleic acid, antibody, etc.), refers to a binding reaction that is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110: 4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett 4: 395; Jeffs et al. (1994) J. Biomolecular NMR 34:17; Tetrahedron Lett 37:743 (1996)) and non-ribose backbones, including those described in U.S. Pat. Nos. 5,235,033 and 5,034.506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part 1, chapt 2, Overview of principles of hybridization and the strategy of nucleic acid probe assays, Elsevier, NY (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term “database” refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The phrases “an amount [of an agent] sufficient to maintain changes in gene expression” or “an amount [of an agent] sufficient to induce changes in gene expression” refers to the amount of the “agent” sufficient maintain or induce those changes in the subject organism as empirically determined or as extrapolated from an appropriate model system.

### BRIEF DESCRIPTION OF THE DRAWINGS

FIGS. 1A-1D show micrographs indicating the location of the PKA catalytic subunit staining in NG108-15 cells after a forty eight (48) hour exposure to 200 mM ethanol. The images in FIGS. 1A and 1B were made with a confocal microscope, whereas those in FIGS. 1C and 1D were made with a light microscope with a fluorescein filter, as were the inserts shown in FIG. 1A and FIG. 1B. FIG. 1A shows control cells in which majority of staining exists in the perinuclear Golgi area. FIG. 1B shows test cells exposed to ethanol, for which the staining is primarily within the nuclear region. FIG. 1C shows the reversibility of the distribution of staining upon withdrawal of ethanol from the cells. FIG. 1D shows cells labeled with an anti-Cα antibody solution that had been preabsorbed by purified Cα. No staining resulted, indicating that the stain is specific for Cα.

FIG. 2 is a graph showing the dependence of the percentage of cells exhibiting nuclear staining as compared to Golgi staining on the concentration of ethanol to which the cells have been exposed.

FIGS. 3A-3D are a series of micrographs comparing the movement of the Cα subunit of PKA in cells exposed to ethanol and that of cells exposed to various other agents as indicated.

FIG. 4 shows the variation of the percentage of cells with Golgi staining over time, for cells exposed to ethanol, and for cells treated with forskolin.

FIGS. 5A and 5B show Western blot analysis of the Cα and RI PKA subunits in ethanol-exposed NG108-15 cells.

FIGS. 6A-6C depict immunohistochemical staining of δ-PKC in NG108-15 cells grown in defined medium in the presence or absence of EtOH or of PKC activation by phorbol myristate acetate (PMA).

FIGS. 7A-7C depict immunohistochemical staining of δ-PKC in NG108-15 cells after a four (4) day exposure to 25 mM ethanol. FIG. 7A-7C depict, respectively: control, non-ethanol exposed cells; test cells exposed to 25 mM ethanol; and a control showing the specificity of the staining when the anti-8 antibody is preabsorbed (“Pre.”) with the immunizing peptide before labeling of the cells.

FIGS. 8A-8C depict immunohistochemical staining of ε-PKC in NG108-15 cells grown in defined medium in the presence or absence of EtOH or of PKC activation by PMA.

FIGS. 9A-9C depict immunohistochemical staining of ε-PKC in NG108-15 cells after four (4) day exposure to 25 mM ethanol. FIGS. 9A-9C depict, respectively: control, non-ethanol exposed cells; test cells exposed to 25 mM ethanol; and a control showing the specificity of the staining when the anti-ε antibody is preabsorbed (“Pre.”) with the immunizing peptide before labeling of the cells.

FIGS. 10A-10D depict immunohistochemical staining of PKA RIα in human neutrophils isolated from nonalcoholics and alcoholics. FIG. 10A depicts untreated neutrophils from nonalcoholics, FIG. 10B depicts untreated neutrophils from alcoholics, FIG. 10C depicts neutrophils from nonalcoholics after in vitro exposure of the cells to 200 mM ethanol for 30 minutes, and FIG. 10D depicts neutrophils from alcoholics after in vitro exposure of the cells to 200 mM ethanol for 30 minutes.

FIGS. 11A and 11B depict immunohistochemical staining of PKA Cα in human lymphocytes isolated from nonalcoholics and alcoholics, respectively.

FIG. 12 depicts CRE-regulated luciferase expression in transfected cells exposed to ethanol.

FIG. 13 depicts relative CRE-mediated luciferase activity in the presence (filled squares) or absence (empty squares) of 200 mM ethanol at the indicated times.

FIG. 14 depicts percent increase over control of CRE-luciferase activity after a 4 hour and a 14 hour exposure to 200 mM ethanol in the presence of BW A1434U (10 μM) or Rp-cAMPS (20 μM).

FIG. 15 depicts percent increase over ethanol-unexposed control of CRE-luciferase activity after a 14 hour exposure to ethanol in cells (a) pretreated for 30 min in the absence or presence of the PKA inhibitor H-89 (10 μM.), 5 μM KN-62, 2 μM PD98059, 1 μM U0126 or 50 nM bisindolymaleimide I (GF) or (b) co-transfected with a dominant negative DN-RIα or DN-MEK construct.

FIG. 16A depicts relative CRE-luciferase activity in cells exposed to ethanol for 14 hours and co-transfected with constructs expressing dominant negative CREB (CREB M1). FIG. 16B depicts percent increase in CRE-luciferase activity over ethanol-unexposed control in cells that express the Gal4-CREB fusion protein and Gal4-luciferase. Such cells were pretreated in the absence or presence of 20 μM Rp-cAMPS for 2 h or with 5 μM KN-62 or 50 nM GF for 30 min. and were further incubated with ethanol for 14 hours.

FIG. 17 depicts a model for the interaction of the pathways triggered by treatment with ethanol or dopamine agonists.

FIG. 18 depicts western blot analysis of the localization of the Cα and Cβ catalytic subunits of PKA and the RI and RIIβ regulatory subunits of PKA in NG108-15 cells grown in the presence or absence of ethanol.

FIGS. 19A-19E show that dopamine D2 receptor activation of ethanol causes translocation of PKA Cα in NG108-15/D2 cells. (FIG. 19A), (FIG. 19B) NPA or ethanol-induced PKA Cα translocation. Cells expressing D2 were incubated in the absence or presence of 50 nM of the D2-specific agonist R(−)-2,10 μl-trihydroxy-N-propylnoraporphine hydrobromide (NPA) or 100 mM ethanol for 10 min. Where indicated, cells were preincubated either with the D2 antagonist spiperone (10 μM) for 30 min or 50 ng/ml PTX overnight before adding NPA or ethanol. The data are representative of at least three independent experiments. Staining intensity is indicated by the color bar, with orange indicating most intense staining (Scale bar, 10 μm) (Images are 400×). (FIG. 19C) Western blot analyses of nuclear (N), membrane (M) and cytosolic (Cy) fractions from cells treated in the absence or presence of NPA or ethanol for 10 min. (FIG. 19D) NPA-induced PKA Cα translocation as a function of time. Cells were incubated with 50 nM NPA for the indicated times, and the percentage of cells showing PKA Cα localization in the cytoplasm relative to the total number of cells determined. Cells were scored positive if staining extended more than one nuclear radius away from the nuclear envelope. Data are presented as the mean±S.E.M. (average of 15 cells/field, 3 fields/experiment, n= 3). (FIG. 19E) PKA Cα translocation as a function of NPA concentration. Cells were incubated with the indicated concentrations of NPA for 10 min and the percentage of cells with PKA Cα in the cytoplasm determined as in (D).

FIGS. 20A-20C show that NPA or ethanol-induced translocation of PKA Cα requires cAMP and induces CRE mediated gene expression. (FIG. 20A) Rp-cAMPS inhibition of NPA- and ethanol-induced PKA Cα translocation. NG108-15/D2 cells were pretreated with 20 μM Rp-cAMPS for 1.5 hr prior to incubation with 50 nM NPA or 100 mM ethanol as in FIG. 19. The data are representative of three or more separate experiments. (Scale bar, 10 μm; images are 400×). (FIG. 20B) NPA modulation of cAMP production. Cells were incubated at 37° C. with or without 50 nM NPA for the indicated times. cAMP levels were measured by radioimmunoassay (Gordon et al., 1986). cAMP levels in the absence of NPA did not change over the time course of the experiment. Data shown are the means±S.E.M of four experiments. * p< 0.05, compared to cells at time zero (one way ANOVA and Dunett test)). (FIG. 20C) NPA- and ethanol-induced luciferase gene expression. NG108-15/D2 cells were transiently transfected with a CRE-luciferase construct and preincubated overnight with or without 50 ng/ml PTX for 30 min, with or without 10 μM spiperone or 20 nM Rp-cAMPS, followed by addition of 50 nM NPA or 100 mM ethanol for 10 min. The data are represented as mean±S. E. M. of at least three separate experiments. *P< 0.01, compared with control (one way analysis of variance and Dunnett's test).

FIGS. 21A-21B show that sub-threshold concentrations of NPA and ethanol act synergistically to induce PKA Cα; translocation and CRE-mediated gene expression. (FIG. 21A) Synergy of PKA Cα translocation. NG108-15/D2 cells were incubated for 10 min with 0.5 nM NPA, 25 mM ethanol, or 0.5 nM NPA plus 25 mM ethanol and localization of PKA Cα determined. Control cells were incubated in media. Where indicated, the cells were pre-incubated overnight with 50 ng/ml PTX, for 30 min with 10 μM spiperone or for 1.5 hr with 20 μM Rp-cAMPS and then incubated in the presence of 0.5 nM NPA plus 25 mM ethanol. (FIG. 21B) Synergy of CRE-mediated luciferase activity. As described in FIG. 2C, the cells transfected with CRE-luciferase were preincubated with or without PTX, spiperone or Rp-cAMPS as in (A), followed by addition of 0.5 nM NPA or 25 mM ethanol alone or in combination for 10 min. *p<0.01 compared with control (one-way analysis of variance and Dunnett's test).

FIGS. 22A-22G show that βγ dimers and A2 are required for PKA Cα translocation, CRE-mediated gene expression and synergy. (FIG. 22A) Western blot analysis of AC II and IV in NG108-15/D2 cells. The data are representative of at least three independent experiments. Preabsorption of isozyme-specific antibody with peptide antigen (Gordon et al., 1997) blocked immunostaining by AC II or IV antibody. Whole cell lysates (40 μg of protein) were probed with anti-AC II or anti-AC IV antibody before (lane 2) and after pre-absorption with peptide antigen, (lane 1). (FIG. 22B) Overexpression of the carboxyl terminus of βARK1 (Minigene). Cells were incubated in the absence or presence of NPA (50 nM) or ethanol (100 mM) after transfection with Ad5βARK1 or Ad5 vector control and probed with PKA Cα antibody. Transfection efficiency was greater than 95% when visualizing cells transfected with Ad5GFP. Results are representative of 6 independent experiments. (Scale bar, 10 nm; images are 400×) (FIG. 22C) Overexpression of the AC II QEHA peptide. Cells were incubated in the absence or presence of ethanol (100 mM), NPA (50 nM) or forskolin (1 μM) after transfection with Ad5QEHA or Ad5 vector control and probed with PKA Cα antibody. Results are representative of four independent experiments. (FIG. 22D) Overexpression of dominant negative βγ scavenger peptides. Cells were transfected with Ad5βARK1, Ad5QEHA or Ad5 vector control, incubated in the absence or presence of NPA (0.5 nM) and/or ethanol (25 mM) and probed with PKA Cα antibody. Results are representative of five independent experiments (scale bar, 10 μM; images are 400×). (FIG. 22E) NPA- and ethanol-induced PKA Cα translocation and synergy. NG108-15/D2 cells were preincubated in the presence or absence of 10 μM BW A1434U for 30 min. and further incubated in the presence or absence of NPA and/or ethanol. Results are representative of three independent experiments (scale bar, 10 μm; images are 400×). (FIG. 22F). NPA- and ethanol induced CRE-mediated gene expression. Cells transiently transfected with CRE-luciferase were preincubated overnight with or without Ad5 QEHA, or for 30 min with or without 10 μM BW A1434U followed by addition of 50 nM NPA or 100 mM ethanol for 10 min. (FIG. 22G). Synergy of CRE-mediated gene expression. Transiently transfected cells were preincubated with or without Ad5 QEHA or BW A1434U followed by addition of 0.5 nM NPA or 25 mM ethanol alone or in combination for 10 min.

FIGS. 23A-23D show that the D2 agonist NPA and ethanol cause translocation of PKA Cα in primary cultures of hippocampal neurons. (FIG. 23A) A1, A2, and D2 blockade and PKA translocation. Neurons were incubated in the absence or presence of 50 nM NPA or 75 mM ethanol for 10 min. Where indicated, the cells were preincubated for 30 min with or without the D2 antagonist spiperone (10 μM), the adenosine receptor antagonist BW A1434U (10 μM), the A1 antagonist DPCPX (100 nM) or the A2 antagonist DMPX (10 μM) before and during the experiment. PKA is indicated by green and the neuron-specific marker (NeuN) by red staining, respectively. Results are representative of three independent experiments (scale, 10 μM; image, 400×). (FIG. 23B) Rp-cAMPS, PTX, QEHA and PKA translocation. Neurons were pretreated for 1.5 hours with or without 20 μM Rp-cAMPS or overnight with or without 50 ng/ml PTX or Ad5QEHA and then incubated in the presence or absence of 50 nM NPA or 75 mM ethanol as in FIG. 5A. Data are representative of at least three independent experiments (scale bar, 10 μM; image 400×). (FIG. 23C) The percentage of cells with Golgi, cytoplasmic and nuclear staining of PKA Cα in primary hippocampal neurons. Data are mean±S.E.M. (average of 15 cells/field, 3 fields/experiment, n=3)* p< 0.01, compared with control (one way analysis of variance and Dunnett's test). (FIG. 23D) Synergy of PKA translocation. Neurons were preincubated with or without spiperone, BW A1434 U, PTX or Ad5QEHA as in FIGS. 5A and 5B and further incubated for 10 min with 0.5 nM NPA and/or 25 mM ethanol. Results are representative of three independent experiments, (scale bar, 10 nM; image 400×).

FIGS. 24A and 24B show that expression of the dominant negative βARK1 βγ scavenger peptide in the nucleus accumbens (NAc) reduces ethanol intake by rats. The mean daily consumption of 10% ethanol and of water was measured following bilateral microinjection of the Ad5Bark1 (n= 7) or Ad5LacZ (n=7) viral vector into the nucleus accumbens. FIG. 24A. Mean (+s.e.m.) ethanol intake (g/kg) is presented for the baseline period (average of 7 days prior to vector injection) and 7 and 14 days after vector injection. *significantly different from Ad-LacZ control, p<0.004. # significantly different from Ad5βARK1, Baseline and Day 14 means, p<0.01. FIG. 24B. Mean (+s.e.m.) water intake (mls).

FIG. 25 illustrates the synergy of PKA signaling in a schematic representation of D2 and ethanol-induced PKA Cα translocation and CRE-mediated gene expression. A central role for βγ subunits in mediating Gi/o and Gs signaling pathways is proposed. D2 signaling is indicated by red arrows; ethanol signaling is indicated by blue arrows. Ethanol inhibits adenosine uptake, leading to an increase in extracellular adenosine and subsequent activation of A2. This increases cAMP and activates PKA signaling. D2 activation releases βγdimers, which activate AC II and/or IV. Ethanol also induces the release of βγ dimers directly, bypassing the D2 receptor. The final common pathway is activation of PKA and release of the catalytic subunit from PKA. PKA Cα translocates to the nucleus, phosphorylates CREB, and increases CRE-mediated gene expression.

### DETAILED DESCRIPTION

### I. Introduction

The subject invention pertains to the elucidation of metabolic pathways underlying the cellular response to consumption or chronic consumption of a substance of abuse (e.g. ethanol, opiates, barbiturates, cannabinoids, etc.). In particular, this invention pertains to the discovery that a dopamine receptor (D2) pathway is coupled to an adenosine receptor pathway (a PKA pathway). In particular, it is demonstrated herein that a D2 agonist can activate PKA signaling and/or can act synergistically with an adenosine receptor to activate such signaling.

Without being bound to a particular theory, it is believed that the mechanism involves D2 coupling to Gi/o, release of βγ dimers, activation of AC II and/or IV, translocation of PKA Cα to the nucleus and subsequent PKA-dependent increases in gene expression.

In addition, we have identified a remarkable synergy between D2 and ethanol-induced activation of PKA. Sub-threshold concentrations of NPA or ethanol (or other substances of abuse) that have no effect alone, when added together induce translocation of PKA and activation of CRE-mediated gene expression. Synergy appears to be due to βγ stimulation of AC II and/or IV concomitant with ethanol/A2 activation of AC. The functional significance of PKA translocation induced by subthreshold levels of NPA and ethanol is suggested directly by synergistic increases in CRE mediated gene expression and indirectly by decreases in ethanol consumption caused by expression of a dominant negative βγ scavenger peptide in the nucleus accumbens (NAc).

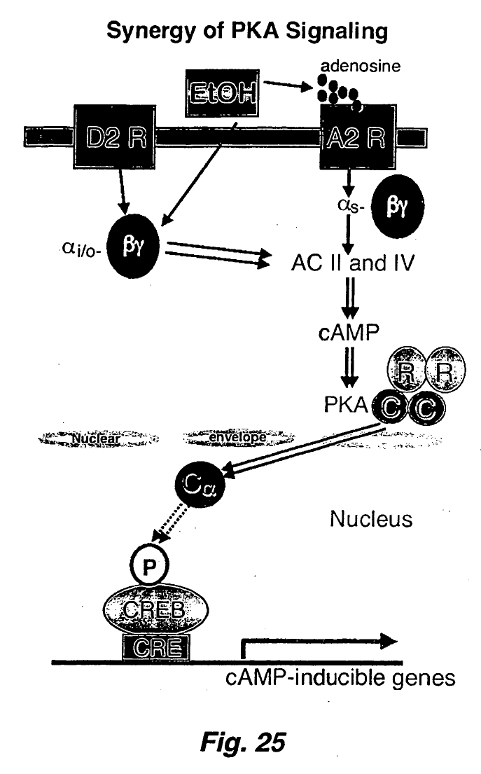

FIG. 25 illustrates a proposed interrelationship between D2 and ethanol activation of PKA signaling. D2 and A2 receptors activate AC via βγ and Gαs, respectively. Ethanol also promotes the release of βγ dimers from Gi/o while activating the A2 Gs-coupled pathway. Increased cAMP levels lead to release of the catalytic subunit(s) from the regulatory subunit(s) of the PKA holoenzyme and translocation of Cα to the nucleus where it phosphorylates CREB and initiates gene transcription from CRE.

The discovery of a synergistic interaction between a dopamine receptor pathway and an adenosine receptor pathway provides new therapeutic modalities for the treatment/amelioration of one or more symptoms associated with chronic consumption of a substance of abuse.

It is believed that during chronic ethanol consumption, adenosine is released activating the A1 and/or A2 receptor(s) and subsequent activation PKA signaling (see, e.g., FIG. 25). The presence of endogenous dopamine acts synergistically, via the D2 receptor, to further augment PKA signaling. Conversely where a substance of abuse other than ethanol is consumed (e.g. a barbituate, an opiate, a cannabinoid, etc.) dopamine levels are increased activating PKA via beta/gamma dimer release from Gi/o. The tonic release of endogenous adenosine synergizes this pathway again resulting in increased PKA activation.

This mechanism can be exploited to treat one or more symptoms associated with chronic consumption of a substance of abuse. Thus, for example, in certain embodiments, this invention contemplates treatment of chronic substance abuse by administering to a mammal (e.g. a human, a non-human mammal including, but not limited to horses, cattle, goats, sheep, canines, felines, largomorphs, rodents, murines, non-human primates, pigs, and the like) an adenosine receptor antagonist and a dopamine receptor antagonist. It is believed that by antagonizing both the dopamine receptor pathway (D2) and the adenosine receptor pathway, the synergistic effect of these pathways can be eliminated. It is believed that by antagonizing both the adenosine receptor pathway and the dopamine receptor pathway, a high level of efficacy can be achieved by significantly lower dosages of adenosine receptor antagonist and/or dopamine receptor antagonist than would otherwise be necessary. This facilitates a treatment regimen that avoids various undesirable side-effects associated with high levels of dopamine receptor antagonists and/or adenosine receptor antagonists. Adverse symptoms associated with the use of dopamine receptor antagonists include, but are not limited to tardive dyskensia, dystonia, and neuroendocrine (hormonal) disturbances. Adverse symptoms associated with the use of adenosine receptor antagonists include, but are not limited to sleep disorders, elevated heart rate, arrhythmia, and the like.

The synergy between the D2 receptor and adenosine receptor pathways can also be used to mitigate one or more symptoms associated withdrawal of a mammal from chronic consumption of a substance of abuse. In such embodiments, a dopamine receptor agonist is administered along with an adenosine receptor (A₁ and/or A₂) agonist. The dosage of one or both agonists (e.g. the combination of agonists) is, in certain embodiments, sufficient to increase activation of PKA, and more preferably bring about cellular changes (e.g. expression of certain genes, expression and/or translocation of certain proteins) characteristic of chronic consumption of a substance of abuse. Because of the synergy between the dopamine receptor pathway and the adenosine receptor pathway, the dosage of dopamine agonist and/or adenosine receptor agonist can be reduced to avoid undesired side effects associated with administration of higher doses of one or both agonists.

The present invention is not limited to compositions and methods that utilize both D2 receptor and adenosine receptor pathways. It was also a discovery of this invention that alterations in activity of PKA can result in behavioral changes associated with chronic consumption of a substance of abuse or associated withdrawal from such chronic consumption. In particular, it was a discovery of this invention that consumptive behavior or craving after withdrawal from a substance of abuse can be reduce or mitigated by increasing effective adenosine levels or activity. This can be accomplished by exploiting the synergy between dopamine D2 receptor activity and adenosine receptor activity, or simply by effectively upregulating adenosine receptor/pathway activity.

This can be accomplished by administering to a mammal an agent that increases effective adenosine levels or activity of an adenosine receptor in a concentration sufficient to mitigate the consumptive behavior or craving. Such agents include, but are not limited to adenosine agonists, adenosine reuptake inhibitors, adenosine deaminase inhibitors or an adenosine kinase inhibitors, and the like.

In certain embodiments, this invention also provides methods of screening for agents that modulate (e.g. upregulate or downregulate) the effect of a substance of abuse on PKA activation in a mammalian cell. In certain embodiments, the screening methods involve contacting a mammalian test cell with a test agent; and detecting the expression or activity of a beta/gamma dimer of (in) the test cell wherein a difference in beta/gamma dimer expression or activity in the test cell as compared to beta/gamma dimer expression or activity in a control cell indicates that the test agent modulates the effect of a substance of abuse on PKA activation.

In other embodiments, this invention relates to discoveries concerning the effects of ethanol or other addictive drugs on the cellular localization and abundance of specific proteins as a consequence of exposure of cells to ethanol or other addictive drugs. Specifically, the invention relates to the discovery that some proteins are predominantly present in different cellular subregions in cells exposed to ethanol or other substances of abuse such as addictive drugs compared to cells that have not been so exposed. Such proteins include, but are not limited to, the Cα catalytic subunit of cAMP-dependent PKC (PKA Cα), the RIα regulatory subunit of PKA (PKA RIα), the RIIβ regulatory subunit of PKA (PKA RIIβ), the α-isozyme of protein kinase C (α-PKC), the δ-isozyme of PKC (δ-PKC), and the ε-isozyme of PKC (e —PKC). Without being bound to a particular theory, the inventors believe that the exposure of certain cells to addictive drugs or other substances of abuse induces translocation of such proteins from one cellular subregion to another.

For example, in EECs of some cell types, (1) the Cα catalytic subunit of cAMP-dependent protein kinase (PKA) (also referred to herein as PKA-Cα) moves from the Golgi region to the nucleus; and (2) the RIα regulatory subunit of PKA (also referred to herein as PKA-RIα) moves from the cytoplasm to the nucleus. It is also believed that in certain cells exposure of cells to ethanol or other substances of abuse induces translocation of the δ-isozyme of PKC from the Golgi region to the perinucleus and the nucleus, while inducing translocation of the ε-isozyme of PKC from the perinucleus to the cytoplasm. As discussed in greater detail below, some of these proteins are localized differently in various cell types and some proteins do not translocate in some cell types. The localization of some proteins useful in the methods of the invention are shown in Table 1.

Additionally, the invention relates to the discovery that the detectable amount of type I (RI) regulatory subunit of PKA found in cells decreases in response to ethanol exposure, while the detectable amounts of α-PKC, δ-PKC, and ε-PKC increase in response to short term as well as long term exposure to ethanol.

The cellular changes in response to exposure to ethanol or other substances of abuse (e.g., addictive drugs) have numerous consequences beyond the effects of ethanol on cellular localization of Cα and δ-PKC and ε-PKC. For example, the dissociation of the catalytic subunit Cα from the regulatory subunit frees the Cα subunit to phosphorylate proteins. Furthermore, upon translocating to the nucleus, the Cα subunit of PKA may phosphorylate a different set of proteins than those available in the Golgi apparatus or elsewhere in the cytoplasm. Moreover, the translocation of the Cα subunit may also alter the extent of phosphorylation of different proteins phosphorylated by Cα, such as CREB, as well as it may alter the CRE-regulated gene expression. The Cα-mediated changes in protein phosphorylation may also have detectable effects on gene expression; such PKA effects may also be used to monitor ethanol exposure. Similar effects of δ-PKC and ε-PKC translocation may be determined. Generally, proteins whose cellular localization or detectable amount are altered by ethanol exposure are referred to herein as ethanol indicative proteins.

The methods of the invention can be employed to monitor withdrawal of ethanol or other addictive drugs from a subject. Specifically, after a chronic alcoholic withdraws from alcohol, it is expected that, in the cell types in which altered localization occurs, Cα and RIα will leave the nucleus and return to the cytoplasm. In some instances, the observed protein will physically return to the origin subregion. Usually, however, the observed protein remains in the destination subregion in the ethanol-exposed cells. But, in newly synthesized, ethanol-naive cells, the protein will be present in the origin subregion, giving the appearance of having “left” the destination subregion. Thus, the techniques described may be used to monitor the withdrawal from ethanol. Aspects of the invention discussed below focus on ethanol as the addictive drug. However, the methods of the invention are readily applicable to other addictive drugs. Exposure to such addictive drugs alters the subcellular localization of a cellular protein. Addictive drugs other than ethanol suitable as subjects for the methods described herein include, but are not limited to, opioids (e.g., heroin), cannabinoids (e.g., marijuana), nicotine, cocaine, addictive drugs that mediate agonist activity at the dopamine D2 receptor, and the like.

### II. Uses of Adenosine and Dopamine Receptor Agonists and Antagonists

In certain embodiments, this invention provides for methods and compositions that mitigate one or more symptoms associated with chronic use of a substance of abuse (e.g., ethanol, opiates, barbiturates, cannabinoids, etc.) or withdrawal from chronic use of a substance of abuse. In certain embodiments, this invention exploits the discovery that dopamine receptor agonists can act synergistically with adenosine receptor agonists to activate PKA. Thus, this invention contemplates the use of dopamine receptor agonists in combination with adenosine receptor agonists to mitigate one or more symptoms (physiological symptoms and/or behavioral symptoms) associated withdrawal or cessation from chronic consumption of a substance of abuse.

In certain other embodiments, this invention contemplates the combined use of dopamine receptor antagonists in combination with adenosine receptor antagonists to block synergistic activation of PKA and thereby to mitigate one or more symptoms (e.g. consumptive behavior) associated with chronic consumption of a substance of abuse.

This invention, however, is not limited to the use of dopamine receptor agonists or antagonists in combination with adenosine receptor antagonists or agonists, but also contemplates the use of adenosine receptor agonists or antagonists alone. It was a discovery of this invention that adenosine receptor antagonists can effect significant behavioral changes on subjects engaged in chronic consumption of a substance of abuse (e.g. ethanol). In particular, it is demonstrated herein, that antagonizing an adenosine receptor can inhibit craving and/or consumptive behavior.

Similarly, the inventors believe that by agonizing (upregulating) and adenosine receptor pathway in mammals undergoing withdrawal from chronic consumption of a substance of abuse cravings and consumptive behavior can be reduced or eliminated.

This invention contemplates the mitigation of one or more symptoms of chronic consumption of a substance of abuse by the use of one or more adenosine receptor antagonists alone, or preferably in combination with one or more dopamine receptor antagonists. This invention also contemplates the mitigation of one or more symptoms of withdrawal from chronic consumption of a substance of abuse (e.g. ethanol) by the use of one or more adenosine receptor agonists alone, or preferably in combination with one or more dopamine receptor agonists.

A) Uses of Antagonists.

In certain embodiments, the methods involve administering to a mammal (e.g., a human, or a non-human mammal) an adenosine receptor antagonist (e.g. an A1 and/or A2 receptor antagonist and/or a non-selective adenosine receptor antagonist) alone, or, in certain embodiments, in combination with a dopamine receptor antagonist (e.g. a selective D2 receptor antagonist and/or a non-selective D2 receptor antagonist). As explained herein, it is believed that the combination of one or more adenosine receptor antagonists with one or more dopamine receptor antagonists permits the use of the receptor antagonists at a lower dosage, while producing greater efficacy in mitigating one or more symptoms associated with chronic consumption of a substance of abuse. Given the synergy between the two active ingredients, the effective amount of each, when administered together, will be less than when either is administered alone. Reduction in effective dosages of the dopamine receptor antagonist(s) and/or the adenosine receptor antagonist(s) permits the use of these agents with fewer characteristic undesired side-effects. The symptoms that can be mitigated include physiological symptoms (e.g. nausea, tremors, cellular distribution or levels of various proteins, etc.) and/or behavioral (e.g. consumptive behavior, depression, anxiety, etc.).

The adenosine receptor antagonists and other substances identified by some of the screening methods described herein, alone and preferably in combination with one or more dopamine receptor antagonist(s) can be used to help addicts refrain from consuming an addictive drug or other substance of abuse. We have demonstrated in vivo efficacy of such antagonists for ethanol and other substances of abuse (e.g. opiates and cannabinoids). Although other mechanisms may apply, the inventors believe that some of the effects of the addictive drug or substance of abuse is due to activation of certain receptors, such as adenosine receptors and/or dopamine receptors. Blocking that activation should reduce the effects of the drug. Therefore, in the presence of the antagonists, the addictive drug/substance of abuse may not give the consumer the desired response. This should decrease the motivation to consume, thereby reducing consumption.

In certain preferred methods, consumption is reduced by administering to an animal (human or non-human mammal) a antagonist(s) that inhibit the effects of the additive drug on the subcellular localization of a protein having the properties discussed herein. Preferably, the animal has chronically consumed the addictive drug but has ceased consumption prior to the administration of the substance. Even more preferably, the animal is not suffering from withdrawal syndrome. Preferably, the substance inhibits the effects of the addictive drug/substance of abuse, or inhibits the effects of a receptor agonist exerting the same effects on subcellular localization as an addictive drug/substance of abuse, in some of the screening methods described herein.

B) Uses of Agonists.

In certain embodiments, the methods involve administering to a mammal (e.g., a human, or a non-human mammal) an adenosine receptor agonist (e.g. an A1 and/or A2 receptor agonist and/or a non-selective adenosine receptor agonist) alone, or more preferably with a dopamine receptor agonist (e.g. a selective D2 receptor agonist and/or a non-selective D2 receptor agonist). It is expected that among other symptoms of withdrawal from such chronic consumption such methods will mitigate/reduce cravings and/or consumption of a substance of abuse (e.g. an addictive drug), preferably ethanol. As discussed herein, various receptor agonists exert the same effects on the subcellular localization of several proteins as does ethanol, such as, for example, adenosine receptor agonists. Moreover, it is shown herein that adenosine receptor agonists and dopamine receptor agonists act synergistic with respect to these cellular effects (e.g. activation of PKA).

Thus, in one embodiment, known adenosine A₁ receptor agonists, and/or adenosine A₂ receptor agonists, and/or non-selective adenosine receptor agonists, and other substances identified by some of the screening methods described herein, alone, but preferably in combination with dopamine receptor agonists can be used to help alcoholics or consumers of substances of abuse (e.g. cannabinoids, opiates, etc) maintain abstinence and/or reduce their consumption of the addictive drug/substance of abuse and/or mitigate other symptoms, particularly physiological and/or behavioral symptoms associated withdrawal.

Without being held to a particular theory, the inventors believe that because such receptor agonists cause some of the same responses as the addictive drug, the agonists can substitute for the addictive drug. This is similar to the use of methadone to maintain abstinence in heroin addicts. In a preferred method, consumption is reduced by administering to an animal, preferably a human, an effective amount of a substance that mimics or enhances the effects of the addictive drug on the subcellular localization of a protein which is distributed differently among the cellular subregions of a DEC versus a DUC. Preferably, the substance mimics or enhances the effects of an addictive drug in some of the screening methods described above.

More particularly, the it is believed that the administration of an adenosine receptor agonist, preferably in combination with a dopamine receptor agonist, during a period of deprivation from consumption of a substance of abuse (e.g. alcohol), will reduce the likelihood of relapse from the cessation. Thus the use of agonists as described herein, will reduce the likelihood of patients relapsing when giving up chronic consumption of a substance of abuse and will facilitate elimination of dependency (physiological and/or psychological) on such substances of abuse.

While, in certain embodiments, adenosine receptor agonists are preferred substances, in other embodiments, a combination of one or more adenosine receptor agonists with one or more dopamine agonist(s) are preferred. As described in greater detail in Example 7, agonists of dopamine D2 receptors, δ-opioid receptors, cannabinoid receptors, adenosine A1 receptors and adenosine A2 receptors each cause the same translocation of PKA-Cα, δ-PKC and ε-PKC as does ethanol. Thus, the administration of any one of these agonists, or in certain embodiments combinations of these agonists, can be used to reduce consumption of ethanol because each mimics the effects of ethanol on translocation of ethanol-indicative proteins. Where the goal is to reduce ethanol consumption by inhibiting its cellular effects, the administration of agonists for at least one of these receptors, more preferably for at least two of these receptors (e.g. adenosine receptor and dopamine D2 receptor) is preferred. Reducing consumption of an addictive drug other than ethanol can similarly be achieved by administering an agonist or antagonist of any of these receptors.

As explained herein, certain embodiments, of the present invention are based on the inventor's discovery that the following pairs of receptor agonists act synergistically upon the translocation of PKA-Cα: dopamine D2 receptor agonists and δ-opioid receptor agonists; and cannabinoid receptor agonists and dopamine D2 receptor agonists, and most particularly D2 receptor agonists and adenosine A1 and/or A2 receptor agonists. The following receptor agonists act in synergy with ethanol to translocate PKA-Cα: dopamine D2 receptor agonists; δ-opioid receptor agonists; and cannabinoid receptor agonists. Thus, consumption of an addictive drug can be reduced by administering two active ingredients, one of which inhibits or activates a receptor that is normally activated by the addictive drug of interest and the other of which inhibits or activates (in same direction as other active ingredient) the other member of a synergistic pair (e.g. a D2 receptor agonist and an adenosine receptor agonist). Given the synergy between the two active ingredients, the effective amount of each, when administered together, will be less than when either is administered alone.

The adenosine agonists used in this invention need not be limited to agonists that act directly via an adenosine receptor. Other agents that increase effective adenosine levels or activity are contemplated alone or in combination with receptor antagonists are contemplated. Thus, for example agents that inhibit adenosine reuptake (e.g. dipyrrimidole) can be used alone and/or in combination with an adenosine receptor agonist, and/or in combination with a dopamine receptor agonist. Similarly, agents that adenosine deaminase inhibitors or an adenosine kinase inhibitors (e.g., erythro-9[2-hydroxyl-3-nonyl]adenine (EHNA), 2′-deoxycoformycin (DCF), 2′-deoxycoformin, 5′amino-5′-deoxyadenosine, 5′-deoxy-5-iodotubercidin, 5′-iodotubericidin, iodotubericidin (16), iodotubericidin, GP515 (17), 4-(N-phenylamino)-5-phenyl-7-(5′-deoxyribofuranosyl)pyrrolo[2,3-d]pyrimidine (GP683)) can be used alone and/or in combination with an adenosine receptor agonist, and/or a dopamine receptor agonist.

C) Methods and Pharmaceutical Compositions for Reducing Consumption of an Addictive Drug.

As explained herein, one or more symptoms associated with the chronic consumption of a substance of abuse (e.g. ethanol) can be mitigated by administration of one or more adenosine receptor antagonists alone, or preferably with the administration of one or more dopamine (D2) receptor antagonists. Similarly, one or more symptoms associated withdrawal from the chronic consumption of a substance of abuse (e.g. ethanol) can be mitigated by administration of one or more adenosine receptor agonists alone or preferably with the administration of one or more dopamine (D2) receptor agonists.

The dopamine receptor antagonists can be administered before, after, or simultaneously with the adenosine receptor antagonists. Similarly the dopamine receptor agonists can be administered before, after, or simultaneously with the adenosine receptor agonists. In certain embodiments, it is preferred that the dosage timing/concentration be sufficient that the effect of the agents on their respective pathways (adenosine receptor pathway/dopamine receptor pathway) at least partially overlaps. This is readily accomplished by coadministration of the active agents and in certain embodiments the active agents are in a single formulation (e.g. a single unit dosage formulation).

Agonists and antagonists of dopamine receptors, opioid receptors, cannabinoid receptors and adenosine receptors are well known to those of skill in the art. For example, without limitation, adenosine receptor antagonists include, but are not limited to caffeine, PD115,199; ZM 241385, quinazoline, 3-(3-hydroxyphenyl)-5H-thiazolo[2,3b]-quinazoline, 1,3-diethyl-8-phenylxanthine, other substituted phenylxanthines, and the like.

Adenosine receptor agonists include, but are not limited to adenosine, adenosine analogues, CGS21680 (Ciba-Geigy), 2-phenylaminoadenosine (CV1808), N,6-cyclohexyl-adenosine, and N,6-cyclopentyladenosine, and the like.

Dopamine receptor agonists include but are not limited to abromocriptine, Bromocriptine, CY 208-243, SKF 83959, ABT-431, SKF 38393, SKF 81297, LY 171555, R(−)-10,11-dihydroxy-N-n-propylnorapomorphine (NPA), and the like.

Dopamine receptor antagonists include, but are not limited to butaclamol, chlorpromazine, domperidone, fluphenazine, haloperidol, heteroaryl piperidines, metoclopramide, olanzapine, perospirone hydrochloride hydrate, phenothiazine, pimozide, quetiapine, risperidone, sertindole, sulpiride, ziprasidone, zotepine, and the like.

Sulpiride is a dopamine receptor antagonist; methadone is an opioid receptor agonist; naltrexone is an opioid receptor antagonist; and caffeine is an adenosine receptor antagonist.

Yet another aspect of the invention is a pharmaceutical composition comprising a substance identified by some of the screening methods described herein. Preferably, the substance is formulated to comprise, in unit dosage form, an amount effective to reduce consumption of ethanol or other addictive drug during the period of time in which the dosage is active in the body of the animal to which it is delivered.

Pharmaceutically useful substances identified by the methods of this invention may be useful in the form of the free acid, in the form of a salt and as a hydrate. All forms are within the scope of the invention. Basic salts may be formed and are simply a more convenient form for use; in practice, use of the salt form inherently amounts to use of the acid form. The bases which can be used to prepare the salts include preferably those which produce, when combined with the free acid, pharmaceutically acceptable salts, that is, salts whose anions are non-toxic to the animal organism in pharmaceutical doses of the salts, so that the beneficial properties inherent in the free acid are not vitiated by side effects ascribable to the cations. Although pharmaceutically acceptable salts of the acid compound are preferred, all salts are useful as sources of the free acid form even if the particular salt per se is desired only as an intermediate product as, for example, when the salt is formed only for purposes of purification and identification, or when it is used as an intermediate in preparing a pharmaceutically acceptable salt by ion exchange procedures.

Such substances can be administered to a mammalian host in a variety of forms, i.e., they may be combined with various pharmaceutically acceptable inert carriers in the form of tablets, capsules, lozenges, troches, hard candies, powders, sprays, elixirs, syrups, injectable or eye drop solutions, and the like depending on the chosen route of administration, e.g., orally or parenterally. Parenteral administration in this respect includes administration by the following routes: intravenous, intramuscular, subcutaneous, intraocular, intrasynovial, transepithelial (including transdermal, ophthalmic, sublingual and buccal), topical (including ophthalmic, dermal, ocular, rectal, nasal inhalation via insufflation and aerosol), and rectal systemic. Oral administration is preferred.

Active compounds may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 6% of the weight of the unit. The amount of active compound in such therapeutically useful compositions is such that a suitable dosage will be obtained. Preferred compositions or preparations according to the present invention are prepared so that an oral dosage unit form contains between about 1 and 1000 mg of active compound.

The tablets, troches, pills, capsules and the like may also contain the following: a binder such as polyvinylpyrrolidone, gum tragacanth, acacia, sucrose, corn starch or gelatin; an excipient such as calcium phosphate, sodium citrate and calcium carbonate; a disintegrating agent such as corn starch, potato starch, tapioca starch, certain complex silicates, alginic acid and the like; a lubricant such as sodium lauryl sulfate, talc and magnesium stearate; a sweetening agent such as sucrose, lactose or saccharin; or a flavoring agent such as peppermint, oil of wintergreen or cherry flavoring. Solid compositions of a similar type are also employed as fillers in soft and hard-filled gelatin capsules; preferred materials in this connection also include lactose or milk sugar as well as high molecular weight polyethylene glycols. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye, flavoring such as cherry or orange flavor, emulsifying agents and/or suspending agents, as well as such diluents as water, ethanol, propylene glycol, glycerin and various like combinations thereof. Of course, any material used in preparing any dosage unit form should be pharmaceutically pure and substantially non-toxic in the amounts employed. In addition, the active compound may be incorporated into sustained-release preparations and formulations.

The active compound may also be administered parenterally or intraperitoneally. For purposes of parenteral administration, solutions in sesame or peanut oil or in aqueous propylene glycol can be employed, as well as sterile aqueous solutions of the corresponding water-soluble, alkali metal or alkaline-earth metal salts previously enumerated. Such aqueous solutions should be suitable buffered, if necessary, and the liquid diluent first rendered isotonic with sufficient saline or glucose. Solutions of the active compound as a free base or a pharmacologically acceptable salt can be prepared in water suitably mixed with a surfactant such as hydroxypropylcellulose. A dispersion can also be prepared in glycerol, liquid polyethylene glycols and mixtures thereof and in oils. Under ordinary conditions of storage and use, these preparations contain a preservative to prevent the growth of microorganisms. These particular aqueous solutions are especially suitable for intravenous, intramuscular, subcutaneous and intraperitoneal injection purposes. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from the previously sterile-filtered solution thereof.

For purposes of topical administration, dilute sterile, aqueous solutions (usually in about 0.1% to 5% concentration), otherwise similar to the above parenteral solutions, are prepared in containers suitable for drop-wise administration to the eye. The therapeutic compounds of this invention may be administered to a mammal alone or in combination with pharmaceutically acceptable carriers. As noted above, the relative proportions of active ingredient and carrier are determined by the solubility and chemical nature of the compound, chosen route of administration and standard pharmaceutical practice. The dosage of the present therapeutic agents which will be most suitable for prophylaxis or treatment will vary with the form of administration, the particular compound chosen and the physiological characteristics of the particular patient under treatment. Generally, small dosages will be used initially and, if necessary, will be increased by small increments until the optimum effect under the circumstances is reached. Oral administration requires higher dosages. The compounds are administered either orally or parenterally, or topically as eye drops. Dosages can be readily determined by physicians using methods known in the art, using dosages typically determined from animal studies as starting points.

Where the adenosine receptor antagonist or antagonist is used in combination with a dopamine receptor antagonist or antagonist, the effective amount of the adenosine receptor antagonist or antagonist is lower than the effective amount of an adenosine receptor antagonist or agonist administered without the dopamine receptor antagonist or agonist. Similarly, in certain preferred embodiments, the effective amount of a dopamine receptor antagonist or agonist is lower than the effective amount of a dopamine receptor agonist or antagonist administered without the adenosine receptor antagonist or agonist. In certain embodiments, the dopamine receptor antagonist or agonist and/or the dopamine receptor antagonist or agonist are administered at a standard therapeutic dosage, more preferably at a substandard therapeutic dosage, still more preferably at about a threshold dosage, and most preferably at a sub threshold dosage, where the threshold dosage or subthreshold dosage is the threshold or subthreshold dosage for the respective agonist or antagonist administered alone. In certain particularly preferred embodiments, the adenosine and/or dopamine receptor agonists or antagonists are administered at a dosage lower than that dosage known to produce one or more adverse side-effects.

### III. Screening and/or Prescreening for Agents that Mitigate One or More Symptoms of Chronic Consumption of a Substance of Abuse or of Withdrawal from Such a Substance

As indicated above, in one aspect, this invention pertains to the discovery that adenosine receptor agonists and dopamine receptor agonists can act synergistically to mediate activation of PKA and associated downstream events (e.g. gene expression) characteristic of chronic consumption of substances of abuse. In certain embodiments, this invention provides methods of screening for antagonists that mitigate one or more symptoms of such chronic consumption or for agonists that mitigate one or more symptoms associated withdrawal from such chronic consumption.

The screening methods can involve screening for an agent that modulates the effect of a substance of abuse on PKA activation in a mammalian cell. It was a discovery of this invention that dopamine receptor synergism of the adenosine receptor pathway can be mediated by beta/gamma dimer activity at adenylate cyclase 2 or 4 in the presence of alpha-s or PKC.

Thus, in certain embodiments, the screening methods of this invention can involve contacting a mammalian test cell with a test agent; and detecting the expression or activity of a beta/gamma dimer of said test cell wherein a difference in beta/gamma dimer expression or activity in said test cell as compared to beta/gamma dimer expression or activity in a control cell indicates that said test agent modulates the effect of a substance of abuse on PKA activation. In certain embodiments, the screening methods can also involve detecting alterations of subcellular location of a protein in a cell exposed to the test agent and/or to a substance of abuse and/or detecting cellular events associated (e.g. protein phosphorylation, gene expression, etc.) with the test agent alone or in combination with a substance of abuse.

Expression levels of a gene can be altered by changes in the transcription of the gene product (i.e. transcription of mRNA), and/or by changes in translation of the gene product (i.e. translation of the protein), and/or by post-translational modification(s) (e.g. protein folding, glycosylation, etc.). Thus preferred assays of this invention include assaying for level of transcribed mRNA (or other nucleic acids derived from nucleic acids that encode a polypeptide comprising a beta/gamma dimer), level of translated protein, activity of translated protein, etc. Examples of such approaches are described below. These examples are intended to be illustrative and not limiting.

A) Nucleic-Acid Based Assays.

1) Target Molecules.

Changes in expression levels of a beta/gamma dimers can be detected by measuring changes in mRNA and/or a nucleic acid derived from the mRNA (e.g. reverse-transcribed cDNA, etc.) that encodes a polypeptide comprising a beta/gamma dimmer. In order to measure the beta./gamma dimer expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism or a cell or tissue culture.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part 1. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+mRNA is isolated by oligo dT column chromatography or by using (dT)n magnetic beads (see, e.g., Sambrook et al. Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see, e.g. Innis, et al., (1990) PCR Protocols. A guide to Methods and Application. Academic Press, Inc. San Diego), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 6: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of beta/gamma dimer in a sample, the nucleic acid sample is one in which the concentration of the beta/gamma dimer mRNA transcripts), or the concentration of the nucleic acids derived from the beta/gamma dimer polypeptide mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the sample comprising a nucleic acid encoding a beta/gamma dimmer polypeptide the total mRNA or a total cDNA isolated and/or otherwise derived from a biological sample. The nucleic acid may be isolated from the sample according to any of a number of methods well known to those of skill in the art as indicated above.

2) Hybridization-Based Assays.

Using the known nucleic acid sequences encoding polypeptides comprising beta/gamma dimers, detecting and/or quantifying transcript(s) of these nucleic acids can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed beta and/or gamma dimmer mRNA), typically fragmented and separated on an electrophoretic gel, is hybridized to a probe specific for that nucleic acid. Comparison of the intensity of the hybridization signal from the “test” probe with a “control” probe (e.g. a probe for a “housekeeping gene) provides an estimate of the relative expression level of the target nucleic acid.

Alternatively, the mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target mRNA. Appropriate controls (e.g. probes to housekeeping genes) provide a reference for evaluating relative expression level.

An alternative means for determining the beta/gamma dimmer expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

3) Amplification-Based Assays.

In another embodiment, amplification-based assays can be used to measure beta/gamma dimmer expression (transcription) level. In such amplification-based assays, the target nucleic acid sequences (i.e., a nucleic acid encoding a beta/gamma dimmer polypeptide or fragment thereof) act as template(s) in amplification reaction(s) (e.g. Polymerase Chain Reaction (PCR) or reverse-transcription PCR (RT-PCR)). In a quantitative amplification, the amount of amplification product will be proportional to the amount of template (e.g. beta/gamma polypeptide-encoding mRNA) in the original sample. Comparison to appropriate (e.g. healthy tissue or cells unexposed to the test agent) controls provides a measure of the transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y.

4) Hybridization Formats and Optimization of Hybridization Conditions.

a) Array-based hybridization formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high-density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high-density arrays is also described in U.S. Pat. Nos. 5,744,305, 5,800,992 and 5,445,934.

b) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides as described herein.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g. down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, die wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of beta/gamma dimmer expression levels can be full length or less than the full length of the polypeptides comprising the beta/gamma dimer. Shorter probes are empirically tested for specificity. Preferred probes are sufficiently long so as to specifically hybridize with the target nucleic acid(s) under stringent conditions. The preferred size range is from about 20 bases to the length of the target mRNA, more preferably from about 30 bases to the length of the target mRNA, and most preferably from about 40 bases to the length of the target mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens that can be employed include those molecules and compounds which absorb light in a distinctive range of wavelengths so that a color can be observed or, alternatively, which emit light when irradiated with radiation of a particular wave length or wave length range, e.g., fluorescein.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, lucifetins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-Based Assays.

1) Assay Formats.

In addition to, or in alternative to, the detection of nucleic acid expression level(s), alterations in expression or activity of a beta/gamma dimer can be detected and/or quantified by detecting and/or quantifying the amount and/or activity of a translated beta/gamma dimer polypeptide.

2) Detection of Expressed Protein

The polypeptide(s) comprising a beta/gamma dimer can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, a beta/gamma dimer polypeptide is detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of a beta/gamma dimer. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to a domain of the antibody.

In preferred embodiments, a beta/gamma dimer polypeptide is detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (beta/gamma polypeptide(s)). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111:1401-1406, and Akerstrom (1985) J. Immunol., 135:2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (beta/gamma dimer polypeptide) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and die sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind beta/gammer dimer polypeptide(s), either alone or in combination. In the case where the antibody that binds the target polypeptide(s) is not labeled, a different detectable marker, for example, an enzyme-labeled antibody capable of binding to the monoclonal antibody which binds the target polypeptide, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutinatidn with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds beta/gamma dimer polypeptide(s) is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein are commercially available or can be produced as described below.

3) Antibodies to Beta/Gamma Dimer Polypeptides.

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides.

If desired, the immunizing peptide may be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g. Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

Preferably, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′}, and/or single-chain antibodies (e.g. scFv) which are capable of binding an epitopic determinant. Also, in this context, the term “mab's of the invention” refers to monoclonal antibodies with specificity for a beta/gamma dimer.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mAb's can be accomplished using relatively routine screening techniques (such as the enzyme-linked immunosorbent assay, or “ELISA”) to determine the elementary reaction pattern of the mAb of interest.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1:M to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

C) Assay Optimization.

The assays of this invention have immediate utility in screening for agents that modulate the beta/gamma dimer expression and/or activity in a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription of a beta/gamma dimer nucleic acid, nucleic acid based assays are preferred.

Routine selection and optimization of assay formats is well known to those of ordinary skill in the art.

D) Pre-Screening for Test Agents that Bind Beta/Gamma Dimer Nucleic Acids or Beta/Gamma Dimer Polypeptide(s).

In certain embodiments it is desired to pre-screen test agents for the ability to interact with (e.g. specifically bind to) a nucleic acid that encodes a polypeptide comprising a beta/gamma dimer (a beta/gamma dimer nucleic acid) or to a beta/gamma dimer polypeptide. Specifically, binding test agents are more likely to interact with and thereby modulate beta/gamma dimer expression and/or activity. Thus, in some preferred embodiments, the test agent(s) are pre-screened for binding to beta/gamma dimer nucleic acids or to beta/gamma dimer proteins before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the beta/gamma dimer protein or nucleic acid is immobilized and exposed to a test agent (which can be labeled), or alternatively, the test agent(s) are immobilized and exposed to a beta/gamma dimer protein or to a beta/gamma dimer nucleic acid (which can be labeled). The immobilized moiety is then washed to remove any unbound material and the bound test agent or bound beta/gamma dimer nucleic acid or protein is detected (e.g. by detection of a label attached to the bound molecule). The amount of immobilized label is proportional to the degree of binding between the beta/gamma dimer protein or nucleic acid and the test agent.

E) Scoring the Assay(s).

The assays of this invention are scored according to standard methods well known to those of skill in the art. The assays of this invention are typically scored as positive where there is a difference between the activity seen with the test agent present or where the test agent has been previously applied, and the (usually negative) control. In certain preferred embodiments; the change/difference is a statistically significant change/difference, e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the difference/change is statistically significant at a greater than 80%, preferably greater than about 90%, more preferably greater than about 98%, and most preferably greater than about 99% confidence level. Most preferred “positive” assays show at least a 1.2 fold, preferably at least a 1.5 fold, more preferably at least a 2 fold, and most preferably at least a 4 fold or even a 10-fold difference from the negative control.

F) Agents for Screening: Combinatorial Libraries (e.g., Small Organic Molecules).

Virtually any agent can be screened according to the methods of this invention. Such agents include, but are not limited to nucleic acids, proteins, sugars, polysaccharides, glycoproteins, lipids, and small organic molecules. The term small organic molecules typically refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library such as a polypeptide (e.g., mutein) library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) J. Med. Chem. 37(9): 1233-1250).

Preparation of Combinatorial Chemical Libraries is Well Known to Those of Skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al. (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include, but are not limited to, automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist and the Venture™ platform, an ultra-high-throughput synthesizer that can run between 576 and 9,600 simultaneous reactions from start to finish (see Advanced ChemTech, Inc. Louisville, Ky.)). Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

G) High Throughput Screening

Any of the assays for compounds modulating the accumulation or degradation of metabolic products described herein are amenable to high throughput screening. Preferred assays detect increases or decreases in beta/gamma dimer transcription and/or translation in response to the presence of a test compound.

The cells utilized in the methods of this invention need not be contacted with a single test agent at a time. To the contrary, to facilitate high-throughput screening, a single cell may be contacted by at least two, preferably by at least 5, more preferably by at least 10, and most preferably by at least 20 test compounds. If the cell scores positive, it can be subsequently tested with a subset of the test agents until the agents having the activity are identified.

High throughput assays for various reporter gene products are well known to those of skill in the art. For example, multi-well fluorimeters are commercially available (e.g., from Perkin-Elmer).

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detectors) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

H) Modulator Databases.

In certain embodiments, the agents that score positively in the assays described herein (e.g. show an ability to modulate beta/gamma dimer expression) can be entered into a database of putative and/or actual modulators of beta/gamma dimer expression or modulators of an organisms response to a substance of abuse or to withdrawal therefrom. The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

### IV. Methods Relating to Alteration of Subcellular Location of a Protein in a Cell Exposed to an Addictive Drug

In the embodiments described below, methods are explained for examining the effects of ethanol and other addictive drugs and screening for therapeutic agents which affect these effects either in vivo or in vitro. It will be readily apparent to one skilled in the art that these techniques may be applied to a number of problems.

The invention is not intended to be limited to the detection of the proteins mentioned above, but one skilled in the art can use other proteins whose behavior in the presence of ethanol or other addictive drugs is similar to those described above. It is known in the art that certain proteins, e.g., PKA Cα, translocate in response to some cellular stimulus. Using the techniques described herein one of skill in the art can identify those proteins that also translocate in response to cellular exposure to ethanol or other addictive drugs. Other proteins that may translocate upon various cellular stimuli include, but are not limited to, various isozyme-specific receptors for activated PKC (RACKs), such as a δ-PKC specific RACK, RACK 2 (also referred to as β′ COP, which is ε-PKC specific), various scaffolding proteins, such as AKAP 79 (A. Kinase Anchoring Protein) (see, for example, Disatnik et al., Exp. Cell Res (1994) 210:287-97; Mochly-Rosen et al., Science (1995) 268:247-251; Mochly-Rosen et al., Biochem Soc Trans. (1995) 23:596-600; Pawson et al., Science (1997) 278:2075-2080). Accordingly, the scope of the invention is not to be limited to the described embodiments, but rather includes methods that involve detecting any protein localized in different cellular subregions or in a different amount in a given cellular subregion in drug exposed cells (DECs) compared to drug unexposed cells (DUCs), or in cells exposed to other addictive drugs compared to unexposed cells.

The localization of various proteins can be determined in a variety of ways as described below. Generally, cells are examined for evidence of (1) a decrease in the amount of the protein in an origin cellular subregion; (2) an increase in the amount of the protein in a destination cellular subregion (or in an intermediate destination cellular subregion); or (3) a change in the distribution of the protein in the cellular subregions of the cell. The evidence can be direct or indirect. An example of indirect evidence is the detection of a cellular event mediated by the protein including, but not limited to, the cellular events discussed below.

A) Detecting Subcellular Distribution of a Protein.

Determination of the localization of the RIα-subunit of PKA, RIIβ-subunit of PKA, Cα-subunit of PKA, δ-PKC, ε-PKC, or any other ethanol indicative proteins or other proteins which are localized differently in cells exposed to other addictive drugs can be carried out in any of a number of ways. A preferred way is by detection of a colorimetric change, for example, by visual observation. Various methods of visual observation can be used, such as light microscopy, fluorescence microscopy, and confocal microscopy. If desired, an epifluorescence microscope with a CCD camera can be used to measure translocation in the assays described below. This procedure may be automated, for example, by computer-based image recognition. The intracellular distribution of the protein can be determined by staining a cell with a stain specific for the protein. The stain comprises a specific binding substance which binds specifically to the targeted protein. Examples of such a stain include, but are not limited to, antibodies that specifically bind to the protein. A stain specific for, e.g., the ethanol-indicative protein, such as for PKA Cα, PKA RIα, PKA RIIβ, α-PKC, δ-PKC or ε-PKC can be prepared using known immunocytochemistry techniques. Stains specific for other proteins having cellular locations or quantities that may be correlated with ethanol or other addictive drug exposure may be similarly prepared. Preferably, the stain further comprises a labeling moiety. Suitable antibodies may be prepared using conventional antibody production techniques. The antibodies may be monoclonal or polyclonal. Antibody fragments, such as, for example Fab fragments, Fv fragments, and the like, are also contemplated. The antibodies may also be obtained from genetically engineered hosts or from conventional sources. Antibodies may be prepared in response to the ethanol indicative protein, e.g., Cα, α-PKC, δ-PKC, ε-PKC, RIIβ or RIα, or immunologically reactive fragments thereof. Techniques for antibody production are well known to the person of ordinary skill in the art and examples of such techniques can be found in Harlow and Lane, Antibodies: A Laboratory Manual. Cold Spring Harbor Laboratory Press, Cold Spring Harbor (1988), Birch and Lennox, Monoclonal Antibodies: Principles and Applications. Wiley-Liss, New York (1995). Examples of suitable antibodies include, but are not limited to, those available from Santa Cruz Antibodies, preferably Lot Nos. B095, B266, G275 and D047 of antibodies that recognize ε-PKC, Lot Nos. 1225 and B226 of antibodies that recognize δ-PKC, and Lot No. 1267 of antibodies that recognize PKA-Cα The labeling moiety will be visibly observable in conventional immunohistochemical detection techniques being, for example, a fluorescent dye such as fluorescein, a chemiluminescence reagent, a radioisotope, a colloidal label, such as colloidal gold or colored latex beads, an enzyme label, or any other known labeling complex. Such stains may be prepared by conventional techniques, for example as described in Manson (1992) Immunochemical Protocols Methods in Molecular Biology Vol. 10, Humana Press, Totowa, N.J., and Beesley (1993) Immunocytochemistry: A Practical Approach, IRL Press, Oxford, England.

Fusion proteins can also be used to track the localization of a protein. The fusion partner can be detectable directly, such as the green fluorescent protein (GFP), or can be detected indirectly using antibodies specific for the fusion partner or by detecting the enzymatic products of a fusion partner such as β-galactosidase. Cells which express a fusion protein can be prepared by transfecting a host cell with a polynucleotide encoding the fusion protein. Preferably, the fusion protein is expressed at levels low enough to avoid expression in vast excess of other cellular factors which may be required for subcellular localization of the protein. For example, if a 100-fold molar excess of the fusion protein is expressed relative to a factor required for translocation from the origin subregion to the destination subregion, translocation upon exposure to, e.g., ethanol, may not be detectable because most of the fusion protein would remain unbound in the origin subregion. This goal can be achieved by not using strong promoters, enhancers or origins of replication giving rise to high copy numbers of plasmids, and by transfecting with smaller amounts of DNA. Preferred fusion proteins include GFP fused to a protein for which its localization is of interest, such as, for example, PKA Cα, PKA RIIβ, δ-PKC, and ε-PKC. GFP can be fused to either the amino terminus or the carboxy terminus of the protein of interest. A tag, such as a histidine tag, may be included, if desired.

Another preferred way to detect a colorimetric change is to use more than one stain. Preferably, the combination of the stains results in a different color than either stain alone. For example, a cell can be stained with a first stain specific for a particular cellular subregion to be examined and a second stain specific for a particular ethanol indicative protein that migrates to or from that cellular subregion in a cell exposed to ethanol. Examples of such staining systems are known in the art and can be adapted for use in the methods described below. A preferred staining system involves the use of a fluorescence indicator, such as, for example, fluorescein, Cy3, Cy5, Texas Red, rhodamine, and the like. For example, ethanol-treated cells can be stained with antibodies to PKA Cα and secondary antibodies conjugated to fluorescein, which would stain the nuclei green. If the cells are further stained with a red nuclear-specific dye (such as, for example, TOTO-3), the nuclei with PKA Cα will appear yellow instead of red. Other dyes for specific cellular subregions include, but are not limited to, Golgi markers such as mannosidase II and BODIPY TR ceramide (Molecular Probes), nuclear markers such as Neu N, and conjugated antibodies recognizing proteins specific to a particular subregion such as Golgi marker enzymes, histones, and the like.

The particular protein and cellular subregion(s) selected for examination can vary depending on the cell type to be used in a particular method. Cells used in the methods of the invention are of a cell type in which the selected protein is predominantly present in a different amount in a particular cellular subregion of ethanol-exposed cells compared to ethanol-unexposed cells. A difference in intracellular distribution of a particular protein in ethanol-exposed cells can be observed in certain cell types but not others. For example, PKA Cα translocates from the cytoplasm to the nucleus in cultured neural cells, e.g., the neuroblastoma/glioma cell line NG108-15, exposed to ethanol for 10 minutes or for 12-48 hours, CHO cells exposed to ethanol for 10 minutes, lymphocytes exposed to ethanol for 30 minutes or for 12-48 hours, and brain Purkinje cells exposed to ethanol for 72 hours, but not in neutrophils. Table 1 provides a list of the observed intracellular distribution of some ethanol indicative proteins in some cell types. One of skill in the art can readily determine the intracellular distribution of other ethanol indicative proteins by staining ethanol-exposed and ethanol-unexposed cells of various cell types with stains (e.g., antibodies) specific for such proteins. Other useful ethanol indicative proteins are likely to be proteins that are known to translocate in response to some cellular stimulus, such as, for example, activation by cAMP, cGMP, phorbol myristate acetate (PMA), phosphorylation, or inactivation by dephosphorylation. Most preferred are kinases and phosphatases, such as, for example, calcium/calmodulin dependent kinases, and protein phosphatases 1, 2A, 2B, and MAP kinase. Other proteins that translocate include calcium/calmodulin and transcription factors.

TABLE 1
Localization of Ethanol - Indicative Proteins

| | Ethanol- | Cellular Subregion in which the | |
|---|---|---|---|
| | Indicative | Protein is Predominantly Present | |
| Cell Type | Protein | EUC (Origin) | EEC (Destination) |
| NG108 | PKA Cα | Golgi | Nucleus &cytoplasm |
| | | | (10′ exposure) |
| | | | Nucleus |
| | | | (12-48 hr exposure) |
| | | | Perinucleus and nucleus |
| | δ-PKC | Golgi | Cytoplasm |
| | ε-PKC | Perinucleus | Nucleus |
| | RIIβ | Golgi | Cytoplasm |
| | RACK 2 | Perinucleus | Cytoplasm |
| | (β′COP) | | |
| | Protein | Nucleus | |
| | Phosphatase | | |
| | I and IIB | | |
| Lymphocyte | PKA Cα | Cytoplasm | Nucleus |
| | PKA RIα | Cytoplasm | Nucleus |
| | PKA RIIβ | Cytoplasm | Cytoplasm |
| | δ-PKC | Cytoplasm | Cytoplasm |
| | ε-PKC | Cytoplasm | Cytoplasm |
| Neutrophil | PKA Cα | Cytoplasm | Cytoplasm |
| | PKA RIα | Cytoplasm | Nucleus |
| CHOp | PKA Cα | Golgi | Cytoplasm |
| | | | (10′ exposure) |
| | | | Nucleus |
| | | | (12-48 hr exposure) |
| | δ-PKC | Golgi | Perinucleus and nucleus |
| | ε-PKC | Perinucleus | Cytoplasm |
| | | | (10′ exposure) |
| | RACK 2 | Perinucleus | Cytoplasm |
| | (β′COP) | | (10′ exposure) |
| Purkinje | PKA Cα | Cytoplasm | Not detectable |
| cells in | | | (72 hr exposure) |
| brain section | ε-PKC | Cytoplasm | Not detectable |
| | | | (72 hr exposure) |

*Unless otherwise indicated, the protein is predominantly present in the destination cellular subregion in cells exposed to ethanol for either 10 minutes or for 12-48 hours.

B) Detecting Cellular Events Induced by an Ethanol-Indicative Protein.

A change in the cellular localization of a protein in a cell exposed to ethanol or other addictive drugs can trigger certain cellular events that can be detected. Examples of such events include phosphorylation of substrate proteins, gene regulation, changes in cytoskeletal structure, release of synaptic vesicles and the like. Such cellular events can be examined in a variety of ways as discussed in greater detail below.

1. Phosphorylation.

Another aspect of the invention is to provide methods for detecting the effects of ethanol or other addictive drugs on cells by measuring the phosphorylation of proteins that are differentially phosphorylated in the presence and absence of ethanol. As previously discussed, exposure of cells to ethanol results in the translocation of Cα to the nucleus, where the Cα catalytic subunit may phosphorylate serine and/or threonine targets in a set of proteins that differs from the set of proteins available for phosphorylation in the cytoplasm, plasma membrane or Golgi. One such protein is CREB.

The identity of other proteins that are differentially phosphorylated in response to ethanol exposure may readily be determined using conventional assay techniques known to the person of skill in the art. For example, radioactively labeled phosphate may be added to cultured cells grown in both the presence and absence of ethanol. Proteins from the labeled cells may then be extracted and separated on a one or two dimensional gel system. Isolated phosphorylated proteins may then be visualized by autoradiography and related techniques. After separation and visualization, changes in the level of phosphorylation of different proteins may be determined by comparing the results obtained from cells exposed to ethanol with the results obtained from cells not exposed to ethanol. Preferably, proteins of interest are immunoprecipitated. Proteins that are differentially phosphorylated by an ethanol indicative protein in response to ethanol may be identified by amino terminus amino acid residue sequencing.

A more sensitive detection method involves the use of phosphoantibodies, for example, antibodies that recognize phosphorylated forms of specific proteins, or antibodies that recognize a phosphorylated amino acid residue, such as phosphothreonine or phosphoserine antibodies. Another useful detection method is back-phosphorylation, which is safer than direct phosphorylation assays but less sensitive. Cell extracts are incubated with radiolabeled ATP and Mg⁺⁺ and subjected to gel electrophoresis. Since ethanol alters phosphorylation, a different amount of radiolabeled phosphate will be incorporated into individual proteins of cells exposed to ethanol than in cells which have not been so exposed, resulting in a different pattern of bands on a gel.

Proteins that are differentially phosphorylated in response to cellular ethanol exposure may be used in assays for the exposure of cells to ethanol. Furthermore, these differentially phosphorylated proteins may be used as the targets when screening for compounds that modulate the cellular effects of ethanol. Such assays include assays involving the steps of measuring the phosphorylation of differentially phosphorylated proteins. Compounds could be screened by measuring their effects on phosphorylation of these differentially phosphorylated proteins.

Phosphorylation of such proteins by an ethanol indicative protein in response to cellular exposure to ethanol can be determined in a variety of ways known in the art, such as, for example, by using phospho-specific antibodies specific for various proteins in the signal transduction pathway. Such antibodies are available commercially (e.g. New England Biolabs (NEB), Inc., 32 Tozer Road, Beverly, Mass. 01915). Examples of suitable phospho-specific antibodies include, but are not limited to, anti-phospho CREB antibodies, such as anti-phospho CREB (Ser 133) polyclonal antibody (NEB #9192).

2. Gene Expression

Some proteins which are localized differently in cells exposed to ethanol or other addictive drugs can affect gene regulation, either directly or indirectly. For example, upon migration away from the Golgi, PKA Cα causes the phosphorylation and activation of CREB, which regulates the expression of genes having regulatory regions which contain a cAMP response element (CRE). CRE-regulated genes include, but are not limited to, genes encoding receptors for γ-aminobutyric acid (GABA receptors) and receptors for N-methyl-D-aspartate (NMDA receptors), which are thought to underlie some behavioral patterns observed in alcoholics. Other factors involved in transcription may also be activated upon migration of PKA Cα. For example, it is thought that, upon binding to phosphorylated CREB, CREB binding protein (CBP) sets the transcription machinery in motion. Other proteins that migrate to the nucleus include δ-PKC, PKA RIIβ, PKA RIα, MAP kinase and calcium/calmodulin dependent kinases, such as CaM kinases I, II and IV. For purposes of the methods described below, the gene is preferably regulated by a factor phosphorylated or dephosphorylated (whether directly or indirectly) by the protein. Phosphorylation or dephosphorylation of the factor alters the expression of the gene. For example, expression can be upregulated by certain factors when such factors are phosphorylated. Some factors, when dephosphorylated no longer increase expression. For example, CREM (CREB modulator) and CaM kinase negatively regulate gene expression by inhibiting CREB mediated expression.

Preferably, the regulated gene is a reporter gene, the expression of which is preferably controlled by CRE. Control of expression by CRE can be enhanced by increasing the number of binding sites for phosphorylated CREB and/or decreasing the number of CRE-independent transcription control sequences present in the vicinity of the reporter gene. Examples of reporter genes, include, but are not limited to chloramphenicol acetyl transferase (CAT) (Alton et al., Nature (1979) 282:864-869), beta-galactosidase, firefly luciferase (deWet et al., Mol. Cell. Biol. (1987) 7:725-737), bacterial luciferase (Engebrecht et al., Proc. Natl. Acad. Sci. USA (1984) 1:4154-4158; Baldwin et al., Biochemistry (1984) 23:3663-3667, alkaline phosphatase (Toh et al., J. Biochem. (1989) 182:231-238; Hall et al., J. Mol. Appl. Gen. (1983) 2:101, and green fluorescent protein (GFP) (Meyer et al., Diabetes (1998) 47(12): 1974-1977), a GFP-luciferase fusion protein (Day et al. Biotechniques 1998 25(5):848-850, 852-854, 856), and other genes encoding a detectable gene product. Detection of gene expression can be achieved in a variety of ways depending on the reporter gene used. For example, a fluorescence or chemiluminescence detection system can be used to detect expression of luciferase and GFP. A CREB-dependent GFP construct is preferred. Alternatively, an antibody that recognizes the gene product encoded by a reporter gene can be used to detect expression of many reporter genes as well as many endogenous CRE-regulated genes. Visual observation of a colorimetric change can be used to detect expression of beta-galactosidase or alkaline phosphatase. A reporter gene can be inserted into the cells by various techniques known in the art and described herein. Transient expression is preferred. However, the reporter gene can be present on a vector that is stably integrated into the genome of the cells.

The expression of genes in response to the presence of an ethanol-indicative protein in the nucleus can be monitored by any of a number of ways known in the art and described herein, such as, for example, by Northern analysis, polymerase chain reaction (PCR), Western analysis, radioimmunoassays (RIA), enzyme linked immunoassays (ELISA or EIA), fluorescence activated cell sorting (FACS) analysis, enzyme-substrate assays such as chloramphenicol transferase (CAT) assays, and the like. Preferably, expression of such genes in response to cellular exposure to an addictive drug is determined by detecting a signal at least about 1.5 times that of control cells which have not been exposed to the addictive drug, preferably greater than about 2×.

C. Methods for Determining Exposure to a Substance of Abuse (e.g. an Addictive Drug).

In one aspect, the present invention provides methods for detecting prior and chronic exposure of a sample to ethanol or other addictive drugs. Such methods are useful for a variety of purposes, such as for determining whether an individual has been actively consuming alcohol or other addictive drugs recently or over an extended period of time. Such determinations can be valuable for monitoring compliance with a rehabilitation program for alcoholism or other addictive disease, or for an extension of a drug testing program in the workplace. In addition, a diagnostic test is useful in evaluating all patients undergoing medical care as ambulatory or hospitalized patients. For example, this could be used in a medical screening clinic since 7-10% of the population are alcoholics. Among hospitalized patients, it is estimated that 25-65% have an alcohol-related diagnosis. Some methods of the invention can be used to distinguish between chronic and current consumption in an individual, usually where there is an undetectable level of the addictive drug in the sample.

The methods for detection of exposure to, e.g., ethanol, fundamentally involve detecting the presence or absence of an ethanol indicative protein in a particular cellular subregion of a test cell in the sample, the test cell being of a cell type in which the protein is present in the cellular subregion in a detectably different amount in an EEC than in an EUC. Usually, the protein is distributed differently between an EEC versus an EUC. This amount is compared to the amount of the protein present in the cellular subregion in a control cell of a control sample which has not been exposed to ethanol. The detection of a difference relative to the control is indicative of ethanol exposure. An alternate control is one comprising a cell that has been exposed to ethanol. The detection of a similar amount of the protein relative to the alternate control is indicative of ethanol exposure. These methods are described below and involve detection methods described in greater detail above.

1. Methods for Determining Cellular Exposure to an Addictive Drug

In one aspect, the present invention provides a method for determining prior exposure to ethanol or other addictive drugs of a sample containing at least one cell, comprising identifying a protein having a localization that is substantially affected by exposure to the addictive drug, and determining the distribution of the protein within the cell, thereby producing an indication of the exposure of the cell to the addictive drug. In one specific embodiment, the protein is PKA Cα or PKA RIα. In another specific embodiment, the protein is the δ- or ε-isozyme of the protein kinase C (PKC). Preferably, the sample contains a plurality of cells, and the determination is carried out for several of the plurality of cells. Preferably, the cells analyzed are derived from blood samples, e.g., lymphocytes, granulocytes, etc. The cell type, protein and drug chosen should be compatible in that the cell type should be one in which the protein to be analyzed translocates in response to the drug of interest. For instance, if one desires to determine cellular exposure to ethanol by determining the localization of δ-PKC or ε-PKC, fibroblasts would be a preferred cell type and human lymphocytes would not be advisable since neither δ-PKC nor ε-PKC translocate in these cells following ethanol exposure. Similarly, it would not be advisable to use human blood samples to determine exposure to cocaine since these cells lack norepinephrine transporters. In contrast, human blood cells do contain opioid, cannabinoid and dopamine receptors and can be used to determine exposure to addictive drugs that act through such receptors. Human blood cells may contain nicotinic receptors that would allow them to be used to determine exposure to nicotine.

The step of identifying preferably comprises staining the cell with a staining complex having specific binding affinity for the protein. The step of determining the location of the protein preferably includes imaging or observing the cell using conventional imaging techniques, e.g., a microscope. Nuclear accumulation of a protein, such as an ethanol indicative protein, e.g., the Cα of PKA, δ-PKC or ε-PKC can also be assessed by observing Cα-, RIα-, RIIβ-, δ-PKC-, or ε-PKC-induced cellular events. For example, it may be possible to observe the chronic activation of CREB transcription factor and other nuclear substrates in response to Cα-activation. Preferably chronic activation of such nuclear substrates is detected by using phospho antibodies that specifically bind to the activated substrates discussed above, or by monitoring the expression of endogenous genes regulated by the nuclear substrates, such as, for example, NMDA receptor, enkephalin, glucose transporters, phosphodiesterase, muscarinic (M3) acetylcholine receptor, β₃-adrenergic receptor, α₁ adrenergic receptor, and the like. Such expression studies should be carried out in cells in which the encoded proteins are normally present. For example, it would not be advisable to use blood cells to assess expression changes of the NMDA receptor or enkephalin because these proteins are not normally present in blood cells. While both α and β adrenergic receptors are present on blood cells, their types would need to be confirmed before using such cells to monitor expression of genes regulated by the β₃-adrenergic receptor or α₁ adrenergic receptor. Some of these proteins may also be regulated by other signaling cascades that are in turn regulated by ethanol. Therefore, although these proteins have CREs and the expression of these proteins would be increased by just increasing the cAMP levels, their expression could by altered by ethanol in a PKA-independent manner as well. Suitable detection methods are described in greater detail above. Methods for determining exposure to ethanol are described in detail below and can readily be applied to determining exposure to other addictive drugs as well.

The invention provides methods for determining exposure of cells to ethanol comprising the step of applying a stain using specific affinity for the ethanol indicative protein, e.g., the Cα-subunit of PKA, or the δ- or ε-PKC isozyme to the sample so as to identify the region or regions of the cell that contain Cα, δ-PKC, or ε-PKC, respectively. After the regions of the cell containing PKA or PKC have been identified, the cell or cells are classified as to the distribution of the stain within the cells, wherein localization of Cα stain in the cell nucleus, δ-PKC in the perinucleus and the nucleus, and G-PKC stain in the cytoplasm is indicative of prior exposure of the cell to ethanol. Cells that contain significant, i.e., greater than control cells, detectable amounts of stain for Cα of PKA in the nucleus, the δ-subunit of PKC in the perinucleus and nucleus, or ε-PKC in the cytoplasm, respectively, are indicative as being exposed to ethanol.

Advantageously, the determination of cellular localization of the ethanol indicative protein, e.g., Cα, δ-PKC or ε-PKC, comprises identifying first and second regions within each cell, and classifying cells as a first type if the protein is predominately present in a first region, and as a second type if the protein is predominately present in the second region. The number of cells of the first type may be compared to the number of cells of the second type for a determined number of cells within a sample. A number dependent on the proportion of cells of the first and second types, usually the ratio or percentage, may be correlated with a control derived from reference data to obtain a qualitative determination of whether exposure to ethanol of the sample has exceeded a certain threshold, or to obtain a semi-quantitative determination of the exposure to ethanol of the sample. In the case of Cα, the first region will preferably be the nucleus of the cell, and the second region will preferably be the perinuclear Golgi apparatus. In the case of δ-PKC, the first region will preferably be the perinucleus and the nucleus of the cell, and the second region will preferably be the perinuclear Golgi apparatus. In the case of ε-PKC, the first region will preferably be the cytoplasm of the cell, and the second region will preferably be the perinucleus and the nucleus of the cell. The control will be of the type of cells being examined, and usually fewer than about 25-35% of the cells display localization in the first region, preferably fewer than about 10-15%, with less than about 1-5% being most preferred. Stated another way, if more than about 25-35% of the cells display localization in the first region, the sample will be positive for localization in the first region.

It will be readily appreciated by one of skill in the art that, depending on the cell type examined, a sample can be classified as positive for ethanol exposure based on lower or higher percentages of cells displaying localization in the first region. One factor affecting the observed percentages is the half-life of a particular cell type. More cells of an immortalized cell line, such as a neuroblastoma cell line, CHO cell line, and the like, are expected to display altered localization than primary cells such as lymphocytes, neutrophils, and the like. Furthermore, the half-life of lymphocytes is about two weeks, whereas the half-life of neutrophils is about eight hours. Thus, the absolute number of cells displaying localization changes can vary depending on the amount of time that has passed since the last exposure to ethanol. However, a detectably greater number of cells exposed to ethanol will display localization in the first region, preferably a statistically significant greater number, more preferably at least about 10% more cells, even more preferably at least about 25% more cells, with more than about 40% cells being most preferred.

The classification step includes the step of identifying the cellular location of the stain and hence the location of the ethanol indicative protein, e.g., the Cα-subunit of PKA, δ-PKC or ε-PKC or any other proteins with comparable localization behavior. The precise means of identifying the cellular location of the ethanol-indicative protein, e.g., Cα, δ-PKC or δ-PKC will vary with label used in the stain. Generally, a variety of methods may be used for each type of label selected. For example, a radioisotope label may be detected through film (autoradiography), Charge Coupled Devices (CCDs) and the like.

When analyzing the results of the subject assays in which a sample containing a plurality of cells is stained with a specific stain for an ethanol indicative protein, e.g., Cα, δ-PKC or ε-PKC, the percentage of cells that show altered location of the ethanol indicative protein must be considered. In some instances, not every ethanol exposed cell in a sample will show altered location of the ethanol indicative protein. However, significantly more cells with altered location of the ethanol indicative protein will be found in multiple cell containing samples that have been exposed to ethanol as opposed to control samples. Furthermore, the percentage of cells showing altered location of the ethanol indicative protein, i.e., translocation to the nucleus in the case of Cα, translocation to the perinucleus and the nucleus in the case of δ-PKC, or translocation to the cytoplasm in the case of ε-PKC, is expected to increase with increasing duration of exposure to ethanol and with increasing amount of ethanol to which the cells are exposed. Statistical analysis may be used to develop quantitative correlations between the percentage of cells in a sample sharing altered location of the ethanol indicative protein and the amount of exposure. Other factors to consider when making such correlations include the age and condition of the source of the cell sample, the particular cell type being analyzed, and the like. The sample may be taken from a live subject, for example a human whose ethanol consumption is to be determined. Alternatively, cells cultured in vitro may be used in those embodiments of the invention that are directed to the monitoring of ethanol in subjects, e.g., screening for therapeutic agents. Where the sample is taken from a live subject, the sample is preferably a blood-sample, containing nucleated cells, such as granulocytes and lymphocytes.

2. Methods for Determining Chronic Cellular Exposure to an Addictive Drug

The invention also provides methods of determining a subject's chronic cellular exposure to ethanol or other addictive drugs. Such methods relate to the discovery in cells obtained from a subject that, unlike cells that have not been chronically exposed to ethanol, cells which have been chronically exposed to ethanol continue to exhibit the effects of ethanol on cellular localization of some ethanol indicative proteins regardless of the presence of ethanol in the sample before it is prepared for analysis (see Example 4).

The methods involve determining cellular exposure to an addictive drug of a sample by detecting the amount of an ethanol indicative protein in a particular cellular subregion(s) of at least one test cell in the test sample as described above, and comparing the results to those obtained with a control sample which has not been exposed to ethanol. Many alcoholics are able to achieve blood ethanol concentrations approaching or exceeding those required in vitro for altered localization. Once achieved, the state of altered localization is maintained for a time even through the blood level of ethanol drops below the threshold level. Thus, when comparing samples from an alcoholic and a nonalcoholic having a blood ethanol level of less than about 200 mM, preferably less than about 25 mM, the alcoholic, but not the nonalcoholic, is expected to exhibit-altered localization.

3. Methods for Determining Consumption of an Addictive Drug.

The consumption of an addictive drug by a subject, particularly a human, could in principle be determined by assaying the cells obtained from the subject. Cells for analysis in the subject assays for addictive drug exposure may come from a variety of locations within the body. Cell containing samples may be obtained from organs or non-organ tissue. Preferably, cell containing samples are obtained from easily removed tissues such as blood and skin. Because of the transient and reversible effects of ethanol on ethanol indicative proteins, e.g., Co, δ-PKC, ε-PKC, it is important that cellular samples be fixed in, e.g., methanol, acetone, formaldehyde or paraformaldehyde prior to analysis, or be analyzed with the assays of the invention as soon as possible after the sample is removed from a subject for analysis. Localization of ethanol indicative proteins or addictive drug indicative proteins such as the PKA Cα or PKA RIα (or other proteins localized differently in cells exposed to addictive drugs compared to unexposed cells) in granulocytes and/or lymphocytes can be investigated, provided that the receptor for the addictive drug of interest is expressed in such cells. Both of these cell types can be conveniently obtained from blood samples. For example, to determine the effect of ethanol consumption in a particular individual, a comparison can be made between the proportion of cells having, e.g., predominately nuclear localization of Cα to that obtained from a reference sample. To monitor progress of the individual over time, a number of samples can be taken, and variations in the localization of staining can be monitored. The technique can be used to determine the effects of a treatment on a live subject, by monitoring changes in the subject when provided with the treatment. For example, since the half-life of lymphocytes is about two weeks, PKA Cα should be detected in the nucleus of only about half as many lymphocytes in a sample taken two weeks after a first sampling if there has been no further exposure to ethanol.

4. Methods for Determining Chronic Consumption of a Substance of Abuse (e.g. an Addictive Drug).

The methods described above for determining chronic cellular exposure to an addictive drug can be applied to determining whether a mammal has been chronically consuming an addictive drug. For example, a mammal, such as a human, that has been chronically consuming ethanol is frequently an alcoholic.

Several methods are used in the art to characterize an individual as having a substance use disorder, e.g., being an alcoholic or other addict. A person suffering from such disorders suffers from substance dependence and/or substance abuse, which are defined in the art as a maladaptive use of the substance, leading to clinically significant impairment or distress, as manifested by certain conditions, occurring at any time in the same 12-month period. Substance dependence is diagnosed by three or more of the following conditions: (1) tolerance; (2) withdrawal; (3) often taking the substance in larger amounts or over a longer period than was intended; (4) persistent desire or unsuccessful efforts to cut down or control substance use; (5) a great deal of time is spent in activities necessary to obtain the substance, use the substance, or recover from its effects; (6) important social, occupational, or recreational activities are given up or reduced because of substance use; (7) the substance use is continued despite knowledge of having a persistent or recurrent physical or psychological problem that is likely to have been caused or exacerbated by the substance. Tolerance is defined by either a need for markedly increased amounts of the substance to achieve intoxication or desired effect, or a markedly diminished effect with continued use of the same amount of the substance. Withdrawal is manifested by the characteristic withdrawal syndrome for the substance, or the same or closely related substance is taken to relieve or avoid withdrawal symptoms. Alcohol withdrawal, for example, is manifested by two or more symptoms of autonomic hyperactivity (e.g., sweating or pulse rate greater than 100); increased hand tremor; insomnia; nausea or vomiting; transient visual, tactile, or auditory hallucinations or illusions; psychomotor agitation; anxiety; and grand mal seizures. Substance abuse is diagnosed by one or more of the following conditions: (1) recurrent substance use resulting in a failure to fulfill major role obligations at work, school, or home; (2) recurrent substance use in situations in which it is physically hazardous; (3) recurrent substance-related legal problems; or (4) continued substance use despite having persistent or recurrent social or interpersonal problems caused or exacerbated by the effects of the substance.

The most preferred method of characterizing an individual as having a substance use disorder is a psychological evaluation, for example, an evaluation using an ICD-10 questionnaire such as the one shown in Table 3. Another preferred method is by determining the level of consumption of ethanol by the individual (Watson et al., J. Clin Psych (1995) 51:676-684; Dawson et al, Addiction (1994) 89:345-350; Hasin et al., Alcoholism: Clin Exp Res (1998) 22-580-584; Dawson, J. Studies on Alcohol (1998) 59:191-197; Tivis et al. Alcoholism: Clin Exp Res (1999) 22:902-907). For example, a human who consumes about 168 drinks in 4 weeks (about 90 g/day) or who has an average daily consumption of about 6 drinks/day can be characterized as an alcoholic. A preferred method comprises obtaining a cellular test sample from the mammal, detecting the amount of an ethanol indicative protein in a particular cellular subregion(s) of at least one test cell in the test sample as described above, and comparing the results to those obtained with a control sample which has not been exposed to ethanol. In one embodiment, the blood alcohol level of this mammal is undetectable by conventional methods.

Preferably, if the sample is a blood sample, the ethanol indicative protein examined is PKA Cα or PKA RIα. The half-life of neutrophils is about 8 hours, whereas the half-life of lymphocytes is about 2 weeks. Thus, as the length of time from the last exposure to ethanol increases, the number of cells exhibiting ethanol-mediated cellular localization effects decreases. A preferred assay examines the amount of PKA Cα present in the nucleus of lymphocytes.

D) Methods for Identifying Substances that Alter or Mimic the Effects of a Substance of Abuse.

Another aspect of the invention is to provide screening methods for substances that can alter or mimic the effects of ethanol or other addictive drugs on the cellular localization of a protein. Substances that alter or mimic the effects of ethanol on cellular localization of proteins would be useful for a variety of purposes. One such purpose is as a research tool to study the role of cellular localization of a protein in various aspects of alcoholism or other addictive disease, such as behavior, dependency, etc. Another purpose is to develop drugs for the treatment of alcoholism or other addictions or the prevention of certain manifestations of the diseases. For example, an inhibitor may permit consumption of alcohol without experiencing some of the negative effects of such consumption. An enhancer or a mimic might substitute for ethanol without the detrimental effects, somewhat analogous to the effects of methadone for a heroin addict.

Substances that can be screened for such activity include elements and compounds. Examples of test compounds include, but are not limited to, inorganic compounds, small organic compounds prepared by combinatorial chemistry, known pharmaceutical compounds, combinatorial peptide libraries, compounds or peptides produced by rational drug design, nucleic acid-based compounds, such as, for example, antisense compounds, antibodies or antibody fragments, polysaccharides, and the like. Compounds known to affect pathways believed to be involved in translocation of ethanol indicative proteins can be tested for their ability to alter or mimic the effects of ethanol. Some preferred classes of compounds include agonists and antagonists of adenosine A1 receptors, adenosine A2 receptors, dopamine D2 receptors, δ-opiate receptors, cannabinoid receptors, PKA, phospholipase C (PLC) and PKC, as well as inhibitors and activators of adenylyl cyclase, of Gᵢ, Gₒ and G_{z} mediated functions and of translocation of PKC (for example, those described in U.S. Pat. Nos. 5,519,003 and 5,783,405) or PKA. Also preferred are compounds which are structurally related to those described above.

The detection methods described above can be used in such screening methods to determine whether the substances tested have the desired activity. Such methods can include assays in which PKA RIα, PKA RIIβ, PKA Cα, α-PKC, δ-PKC or ε-PKC cellular localization is determined and/or measured.

The sample can be a tissue sample, such as neural tissue or skin tissue, and is preferably a cell culture. Cells for use in the subject screening assays for compounds that modulate the effects of ethanol are of a cell type in which an ethanol indicative protein is present in a particular cellular subregion in a detectably different amount in an ethanol-exposed cell than in an ethanol-unexposed cell. The cells may be primary cells derived directly from a subject or may be cells from a cell line. Primary cells are cells which can be propagated for only a finite number of cell divisions, i.e., cells which are not immortal. Some preferred primary cells include neuronal cells, such as, for example, glial cells and Purkinje cells and blood cells such as lymphocytes and granulocytes. Cells for use in the assays of the invention may be obtained from cells cultured in vitro. Preferably, the cells used in the assay are from cell line cells. Immortalized cell lines are preferred for use in the subject screening assays in part because they provide more consistency between assays. Examples of immortalized cell lines include tumor cell lines or cell lines derived from fusion of a non-tumor cell with a tumor cell. More preferably the cells are of a cell type derived from brain or neural tissue, such as a neuroblastoma, particularly NG108-15 neuroblastoma×glioma cells, are from a fibroblast cell line, or are from cell lines derived from lymphocytes or granulocytes, such as, for example, HL-60 cells, Jurkat cells, S49 mouse lymphoma cells, and a Friend human leukemia cell line. Most preferred are cells that can be easily cultured in a defined media, preferably a serum-free media (in part because serum contains factors which can interfere with the assay), and are sufficiently adherent so as to remain attached to a surface during wash steps in the assay, such as, for example, NG108-15 cells or Chinese hamster ovary (CHO) cells which have been adapted to grow well for at least about 48 hours in serum-free media or serum-depleted media, respectively.

A preferred assay for detecting the cellular localization of a particular ethanol-indicative protein in the screening methods of the invention comprises detecting a colorimetric change, as described above. Where such assays involve staining or labeling of the cells with a stain or label specific for the protein the test cells and/or control cells can be stained or labeled before exposure to ethanol and/or the substance if the staining or labeling would not interfere with cellular localization in response to exposure to ethanol and/or the substance. Usually the cells are stained or labeled after exposure to ethanol and/or the substance. In one embodiment, the detection assay comprises observing the distribution of a stain specific for the protein by observing the accumulation of stain in one cellular subregion compared to a second cellular subregion. Alternatively, cells can be examined for the presence of the stain in a particular cellular subregion without regard for the presence of the stain in a different cellular subregion.

In yet another embodiment, the localization of a protein of interest can be observed in cells transfected with a polynucleotide encoding the protein of interest fused to a polynucleotide encoding a marker polypeptide which permits detection of the fusion protein in a particular cellular subregion. Preferably, the cells are stably transfected. For example, visual inspection of, e.g., CHO cells expressing a PKA Cα-GFP fusion protein would reveal fluorescence in the Golgi apparatus of cells not exposed to ethanol and fluorescence in the nucleus of cells exposed to ethanol for 12-48 hours. One could examine these cells for changes in this pattern when treated with a test substance.

In another embodiment, the cells can be stained or labeled with a first substance specific for the target cellular subregion and a second substance specific for a particular ethanol indicative protein. Preferably, the first and second substances are substances that produce a colorimetric change when combined, such as, for example, substances used in fluorescence resonance energy transfer (FRET).

Another preferred detection assay comprises detecting the expression of a gene regulated by cellular events mediated by a particular protein, such as an ethanol indicative protein, as described above. Particularly preferred is an assay detecting the expression of a reporter gene regulated by CREB, such as a heterologous luciferase gene operably linked to a CRE recognition sequence as described in greater detail in Example 6. Another preferred reporter system is one in which cells are co-transfected with a GAL4-CREB fusion construct and with a second construct containing the DNA binding site for GAL4 fused to luciferase as a reporter gene, such as the PathDetect™ trans-acting system (see Example 6). Suitable cells for use in a reporter system include neuroblastoma cells, such as NG108-15, Chinese hamster ovary (CHO) cells, HEK 293 cells, other fibroblast cells, primary cultures of neuronal cells, such as glia or neurons, other primary cells such as lymphocytes, and other cells in which migration of an ethanol indicative protein to the nucleus either promotes or inhibits gene expression, with NG108-15 and CHO cells being most preferred. Other suitable reporter genes are described in greater detail above.

1. Methods for Screening for Substances that Alter the Effects of an Addictive Drug on Cellular Localization

In a preferred embodiment, the invention provides a method for identifying a substance that alters the effects of ethanol or other addictive drugs on the cellular localization of a protein. Typically, the method comprises exposing a test sample comprising at least one test cell to an addictive drug or a receptor agonist exerting the same effects on the localization of a protein as an addictive drug, exposing the test cell to the substance, and detecting the presence or absence of a protein in a particular cellular subregion. This amount can be compared to the amount of the protein present in the corresponding cellular subregion of a control cell of a control sample which has been exposed to the addictive drug but not to the substance. If a different amount of the protein is detected in the cellular subregion in a test cell exposed to the substance and the addictive drug compared to a control cell exposed to the addictive drug alone, the substance is considered to have altered the effects of the addictive drug on the cellular localization of the protein.

In part because of the experimental evidence discussed in Example 7, the inventors believe that addictive drugs may involve the same or similar signal transduction pathways. In addition, as shown in Table 6, agonists of several classes of receptors exert the same effects on the subcellular localization of some proteins as is observed with ethanol. Thus, receptor agonists suitable for the screening methods of the invention include, but are not limited to, adenosine receptor agonists, dopamine receptor agonists, δ-opiate receptor agonists, and cannabinoid receptor agonists.

The test sample can be exposed sequentially or simultaneously to the addictive drug or agonist and the substance. When exposed sequentially, the sample is preferably exposed to the substance before the addictive drug or agonist. Data from the control cell can be obtained during the same experimental period as the test cell. Alternatively, the protein can be detected in the control cell in advance of experiments testing a specific substance or during the course of experiments testing a different substance. Those data can provide a reference standard against which to compare data obtained from experiments testing the effects of various substances.

In a preferred embodiment, the test sample comprises a plurality of test cells and the control sample comprises a plurality of control cells. Where the sample comprises a plurality of cells, the method can comprise detecting a minimum percentage of the test cells having a detectably different amount of the protein in the cellular subregion examined. The percentage is a detectably different percentage from that of control cells having a detectably different amount of the protein in the cellular subregion. Preferably, the substance is considered to have altered the effects of ethanol on the cellular localization if the difference in amount is detected in at least a statistically significant greater number of test cells compared to control cells, preferably greater than 5%, more preferably greater than 15%, even more preferably greater than 25%, with greater than 35%, up to 100%, being most preferred, depending on a variety of factors, including, but not limited to, the detection method used.

The test cells can be exposed to the addictive drug and the test substance either simultaneously or separately. When exposed separately to the addictive drug and the substance, the test cells are preferably exposed to the substance before the drug.

2. Examining the Destination Cellular Subregion.

Depending on the particular ethanol indicative protein, cellular subregion, and cell type studied, the protein can be present in the cellular subregion in a detectably greater amount in, e.g., EECs than in EUCs. This can occur, for example, when the protein translocates to the cellular subregion in cells exposed to ethanol. Usually, the protein has translocated from an origin cellular subregion to a destination cellular subregion such that the protein is present in a detectably greater amount in the destination cellular subregion than in the origin cellular subregion in EECs, as shown in the Examples.

The substance can be considered an inhibitor of the effects of ethanol on the cellular localization of the protein if the amount of protein in the destination cellular subregion of the test cells is less than that of control cells which have been exposed to ethanol but not to the substance. Conversely, the substance can be considered an enhancer of the effects of ethanol on the cellular localization of the protein if the amount of protein in the destination cellular subregion of the test cells is greater than that of the control cells.

A substance can also be classified as an inhibitor or an enhancer depending on the number of test cells in which the protein is present in the first cellular subregion. For example, a substance can be classified as an inhibitor if the protein is present in the destination cellular subregion in a detectably lesser number of the test cells than in the control cells. Conversely, the substance can be classified as an enhancer if the protein is present in the destination cellular subregion in a detectably greater number of the test cells than in the control cells.

3. Examining the Origin Cellular Subregion

Depending on the particular ethanol indicative protein, cellular subregion, and cell type studied, the protein can be present in the cellular subregion in a detectably lesser amount in, e.g., EECs than in EUCs. This can occur, for example, when the protein is no longer detectable in the cellular subregion in cells exposed to ethanol. In some instances, the protein translocates to a second detectable cellular subregion (a destination cellular subregion). In other instances, the protein can be detected in EUCs, but is undetectable by some detection methods in any other cellular subregion in EECs. The protein may have been degraded, may have assumed an altered conformation, or may have translocated to a region of the cell where it is not detectable by the detection method used. This is the case, for example, for PKA Cα and ε-PKC in Purkinje cells of cerebellum tissue sections. These proteins are detectable in the cytoplasm in EUCs, but are not detectable at all by immunofluorescent microscopy in EECs.

The substance can be considered an inhibitor of the effects of ethanol on the cellular localization of the protein if the amount of protein in the origin cellular subregion of the test cells is greater than that of control cells which have been exposed to ethanol but not to the substance. Conversely, the substance can be considered an enhancer of the effects of ethanol on the cellular localization of the protein if the amount of protein in the origin cellular subregion of the test cells is less than that of the control cells.

A substance can also be classified as an inhibitor or an enhancer depending on the number of test cells in which the protein is present in the origin cellular subregion. For example, a substance can be classified as an inhibitor if the protein is present in the origin cellular subregion in a detectably greater number of the test cells than in the control cells. Conversely, the substance can be classified as an enhancer if the protein is present in the origin cellular subregion in a detectably lesser number of the test cells than in the control cells.

4. Methods for Screening for Substances that Mimic the Effects of an Addictive Drug on Cellular Localization.

The invention also provides a method for identifying a substance that mimics the effects of an addictive drug on the cellular localization of a protein. Typically, the method comprises exposing a sample comprising at least one test cell to the substance but not to the addictive drug, and detecting the amount of the protein in a particular cellular subregion. This amount can be compared to the amount of the protein present in the corresponding cellular subregion of a control cell which has been exposed to the addictive drug but not to the substance. If a similar amount or distribution of the protein is detected in the cellular subregion in a test cell exposed to the substance compared to a control cell, the substance is considered to have mimicked the effects of the addictive drug. Data from the control cell can be obtained as discussed above in the previous section.

In a preferred embodiment, the sample comprises a plurality of test cells and a plurality of control cells. Where the sample comprises a plurality of cells, the method comprises detecting a percentage of the test cells having an amount of the protein in a particular cellular subregion being examined or a distribution of the protein similar to that of EECs. Detection of a minimum percentage can be indicative of the substance having mimicked the effects of, e.g., ethanol on the cellular localization of the protein. The minimum percentage is preferably a detectably greater percentage than a percentage of cells of a second control which have not been exposed to either ethanol or the substance.

As discussed above in the previous section, the method can involve examining either the cellular subregion in which more of the protein is present in ethanol-exposed cells, or examining the cellular subregion in which more of the protein is present in ethanol-unexposed cells. The distribution of the protein between different cellular subregions within a cell can also be examined, if desired.

### V. Methods Relating to the Alteration of Cellular Levels of a Protein in a Cell Exposed to an Addictive Drug

In another aspect, the present invention provides a method of measuring the exposure to ethanol or other addictive drugs of a sample containing at least one cell, the method comprising measuring, either quantitatively or qualitatively, the amount of a protein or polypeptide, which amount is dependent on exposure of the cell to ethanol or other addictive drugs, and determining the amount of the protein in the cell. In one preferred embodiment, the polypeptide may be the type I regulatory subunit (RI) of PKA where a reduction, for example a 20% to 50% reduction compared to unexposed control cells, is indicative of exposure to ethanol or other addictive drugs. Alternatively, the detectable amount of the protein heat stable protein kinase inhibitor (PKI) may be measured and correlated with exposure to an addictive drug. In another preferred embodiment, the increase of the detectable amount of the α-, δ-, or the ε-subunit of PKC may be determined and correlated with exposure. The determination may be carried out by any of a variety of measurement methods well known to persons of ordinary skill in the art of molecular biology, such methods include ELISA, radioimmunoassay, western blot analysis, and the like.

Substances can be screened for their ability to alter or mimic the effects of ethanol or other addictive drugs on the cellular levels of a protein, such as an ethanol indicative protein by performing assays in which the quantity of e.g., PKA RI, α-PKC, δ-PKC, ε-PKC, or PKI is measured.

### V. Kits

Another aspect of the invention is to provide kits for carrying out the subject methods. Kits generally contain one or more reagents necessary or useful for practicing the methods of the invention. Reagents may be supplied in pre-measured units so as to provide for uniformity and precision in test results. The invention provides kits for determining the exposure of cells to ethanol by means of measuring the amount of RI, αPKC, δ-PKC, or ε-PKC. These RI, αPKC, δ-PKC, or ε-PKC measurement kits comprise a stain specific for RI, αPKC; δ-PKC, or E-PKC. The RI, αPKC, δ-PKC, or ε-PKC measurement kits may further comprise one or more of the following items' additional reagents for the detection of RI, αPKC, δ-PKC, or ε-PKC complexed with the stain; positive controls for RI, αPKC, δ-PKC, or ε-PKC; negative controls for RI, αPKC, δ-PKC, or ε-PKC; RI, αPKC, δ-PKC, or ε-PKC solutions of known concentration; equipment for obtaining tissue samples, and the like.

Kits for determining the exposure of cells to ethanol or other addictive drugs by means of determining the intracellular localization of, e.g., an ethanol indicative protein, such as, for example, PKA Cα, PKA RIα, PKA RIIβ, α-PKC, δ-PKC or ε-PKC preferably comprise a stain specific for the protein or for a gene product the transcriptional regulation of which is mediated by the protein. Such kits may further comprise one or more of the following items: additional reagents required for the detection of a protein that has complexed with the stain, a reference standard(s), stain specific for a cellular subregion, equipment for obtaining tissue samples, instructions for use, and the like. Suitable reference standards include positive controls, negative controls, photographs of such controls, tabulated or graphed data of such controls, and the like. The kits may further comprise instructions for carrying out the methods described above, preferably printed instructions. For example, the instructions can describe detecting the amount of an ethanol indicative protein in a first cellular subregion of a test cell. Suitable stains include, but are not limited to, stains specific for PKA Cα, PKA RIα, PKA RIIβ, δ-PKC, and ε-PKC.

The invention also provides kits for testing compounds for their ability to modulate cellular responses to ethanol using the assay methods of the invention. Such kits are essentially the same as the kits described above for the measurement of cellular localization or protein levels. However, such kits may further comprise a polynucleotide comprising a reporter gene and/or a host cell line useful for the detection of changes in localization. Suitable cell lines are described above. Preferably the host cell line has been transfected with the reporter gene as discussed above. Preferably, a kit using a stain to (1) detect cellular localization of a protein in a cell exposed to an addictive drug, or (2) identify a substance that alters or mimics the effects of an addictive drug on cellular localization, further comprises printed instructions for comparing the localization of the stain in the test cell to the localization of the stain in a control cell of the same cell type. The control cell can be an EEC or an EUC or both. The printed instructions can include a description of localization of one or more ethanol indicative proteins in one or more different cell types. For example, the instructions can include information in the form of a table, such as Table 1. The instructions can also include photographs of such cells, if desired.

Kits using a stain specific for a gene product, the production of which is regulated by a protein which is localized in a different cellular subregion in an EEC than an EUC (e.g., PKA-Cα), or a polynucleotide encoding the gene product, preferably include printed instructions for comparing the production levels of the gene product in a test cell to the production levels in a control cell. Such kits may also include other reagents necessary for detecting the stain, such as, for example, reagents required for the detection of luciferase activity, such as luciferin, ATP and Co-enzyme A, or, where the stain is a polynucleotide probe, a radioisotope or biotin.

In certain embodiments, the kits comprise a container containing a dopamine receptor antagonist and an adenosine receptor antagonist or a dopamine receptor agonist and an adenosine receptor agonist. The agonists or antagonists can be provided individually or combined, can optionally be provided in a pharmaceutically acceptable excipient and can optionally provided on one or more unit dosage formulations. In certain embodiments, the kits further comprise instructional materials providing preferred dosage regimes, preferably preferred dosage regimes for utilization of an adenosine receptor agonist or antagonist in combination with a dopamine receptor agonist or antagonist. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Ethanol Induced Translocation of the Cα Catalytic Subunit of PKA

NG108-15 cells were plated onto single chamber slides in a defined medium at a density of approximately 40,000 cells/slide. The techniques and media used for growing the cells are not critical, and are known to those skilled in the art. Suitable techniques are described in Gordon et al, 1986, Proc. Natl. Acad. Sci. USA 83:2105. The cells were maintained for an additional forty eight (48) hours in the defined media or the defined media containing various concentrations of ethanol (e.g., 25, 50, 100, 200 mM ethanol). The media were replaced by fresh media (with or without ethanol) daily and the slides were wrapped in parafilm to prevent ethanol evaporation. The cells were fixed with methanol on a cooled surface for two (2) to three (3) minutes, and the slides were then immersed twice for five (5) minutes each in phosphate buffered saline (PBS) on ice. After that, the cells were incubated with blocking buffer (1% normal goat serum in PBS containing 0.1% Triton-X-100) at 4° C. for six (6) to twelve (12) hours and then incubated with primary antibody solution for forty eight (48) hours at 4° C. in a humidified chamber. The primary antibody solution was prepared from primary antibodies raised in response to Cα (available from Transduction Laboratories and other companies) diluted in PBS containing 0.1% Triton X-100 and 2 mg/ml fatty acid free bovine serum albumin. The slides were washed as before and incubated in the appropriate FITC (fluoresceinisothiocyanate)-conjugated secondary antibody diluted in the same solution at 1:1000.

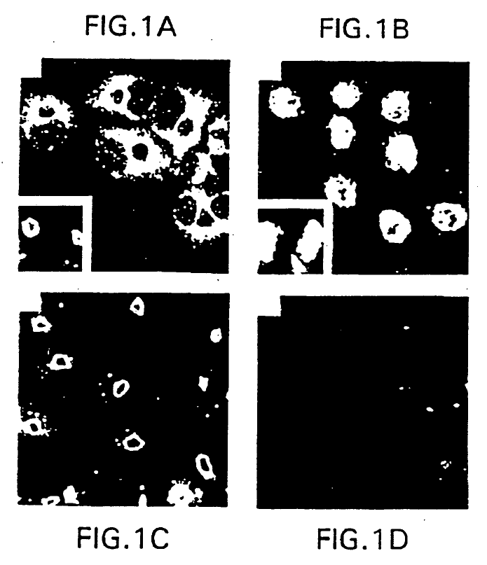

After forty-eight (48) hours, the slides were washed and coverslipped using Vectashield™ mounting medium (Vector Labs). The images in panels A and B of FIG. 1 were made using a BIORAD™ 1024 confocal microscope. The images in panels C and D of FIG. 1 were made using a Leica™ DMBR microscope equipped with a fluorescein filter.

To determine reversibility of exposure to ethanol (results shown in panel C of FIG. 1), the cells were exposed to media containing 200 mM ethanol for forty eight (48) hours then washed three times with fresh media containing no ethanol and incubated without ethanol for an additional forty eight (48) hours. To determine specificity of binding of the stain (results shown in panel D of FIG. 1), 0.1 mg/ml purified catalytic subunit of PKA was added to the primary antibody solution two (2) hours prior to incubation with the fixed cells.

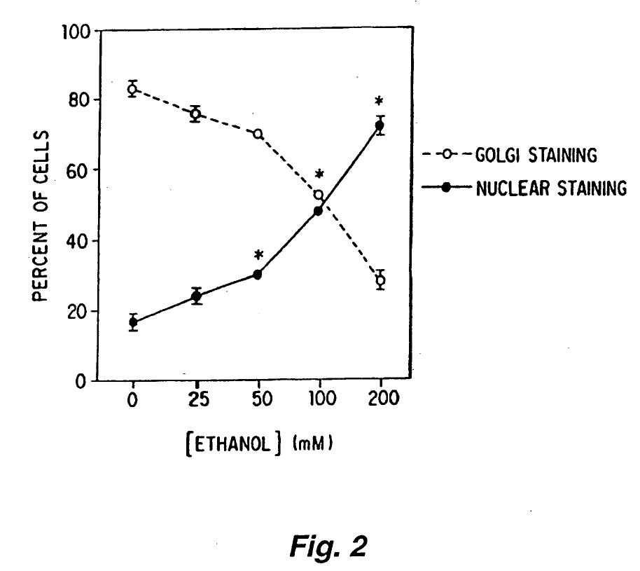

To obtain the numerical data shown in FIG. 2, random fields were selected on the slide and the cells within the field classified as either having primarily Golgi staining or primarily nuclear staining for Cα. Although using only two types of classification simplifies scoring of cells, it may be desirable to classify cells into a plurality of classification groups dependent on the degree of staining in each of several locations. In some instances, it may even be desirable to provide continuous variable classification of each cell, for example, if image intensity is measured at a given point in a cell. At least five (5) fields were classified for a total of at least one hundred (100) cells per slide. The observer was blind to the experimental condition of the slides. Data points are the mean+/−SEM (standard error of the mean copy) of four (4) experiments, *p< 0.05.

The results obtained can be summarized as follows: NG108-15 cells, forming a control which had not been exposed to ethanol, were stained with the specific stain. Cα was found in the perinuclear Golgi area in approximately 80% of the control cells, as depicted in panel A of FIG. 1. In the remaining 20% of the control cells, Cα was found predominately in the nucleus and cytoplasm. This localization of the Cα subunit to the Golgi has been previously observed by Nigg and co-workers. Nigg et al., 1985, EMBO J. 4:2801-2806. Cellular localization of Cα was further confirmed by co-localization with Golgi-specific markers, including mannosidase II, and ceramide. Nigg et al., supra.

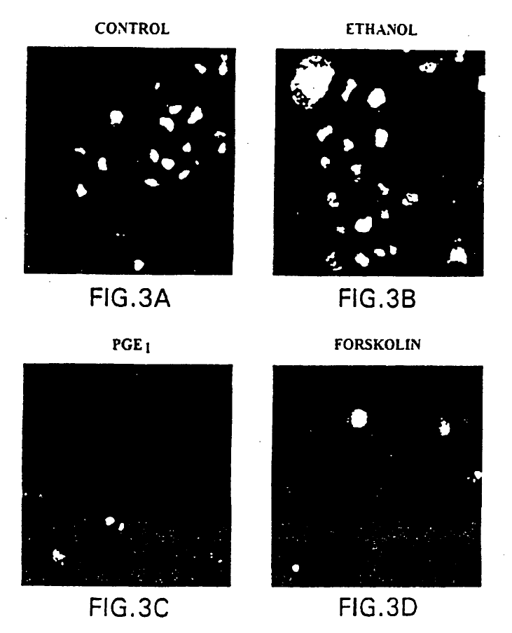

Other cell samples were exposed to ethanol of varying concentrations (25, 50, 100, 200 mM), for a period of forty eight (48) hours, and the assay repeated. The results are shown in FIG. 2. As can be seen from FIG. 2, 75% of cells treated with 200 mM ethanol showed predominant localization of Cα in the nucleus. A micrograph of these cells is shown in panel B of FIG. 3. The results of these investigations serve as a reference to which a sample of cells suspected of being exposed to ethanol can be compared. To provide a screen for a drug or therapeutic agent to discover whether it has any effect on the cellular effects of ethanol, the above procedure can be repeated with the drug present in the growth medium in addition to alcohol. The cells can be pre-incubated with the drug, if desired. Since 100 mM ethanol is found to have a noticeable effect, and 200 mM ethanol is found to have a marked effect on the localization of Cα PKA, a screen for a drug can be provided employing a single growth medium preferably having a ethanol concentration of the order of at least 100 mM, and more preferably of 200 mM, or more. Of course, several growth media containing differing amounts of ethanol may be used, as described above, to investigate efficacy of the drug at varying levels of ethanol concentrations. This may be particularly useful for investigating the effects of a drug on long-term low-level exposure to ethanol.

The results from a control sample can be stored so that when screening for a particular drug, it is not necessary to conduct a control experiment each time. However, it is often desirable to conduct a control experiment whenever a drug is screened, to compensate for variations in other factors which may affect the results.

The above procedure is sufficient to form the basis for a screen. Additional factors having an effect on the localization of PKA Cα and activity of other PKA subunits have been identified. The following information may therefore be of assistance in assessing how the screening method may be affected by external factors.

Reversibility of the localization is demonstrated by panel C of FIG. 1, which is a micrograph of a similar sample forty eight (48) hours after withdrawal from the ethanol. As can be seen, the majority of the Cα had returned to the Golgi apparatus. In a screen for a drug, therefore, it is important to classify the cells relatively soon after removal from the medium, usually within forty eight (48), preferably within twelve (12) hours, preferably immediately. Similarly, samples taken from patients should be classified soon after removal.

To test for specificity of the stain, the stain was preabsorbed by purified Cα prior to staining, and as can be seen from panel D of FIG. 1, virtually no staining resulted, indicating that the polyclonal antibody stain is specific to Cα.

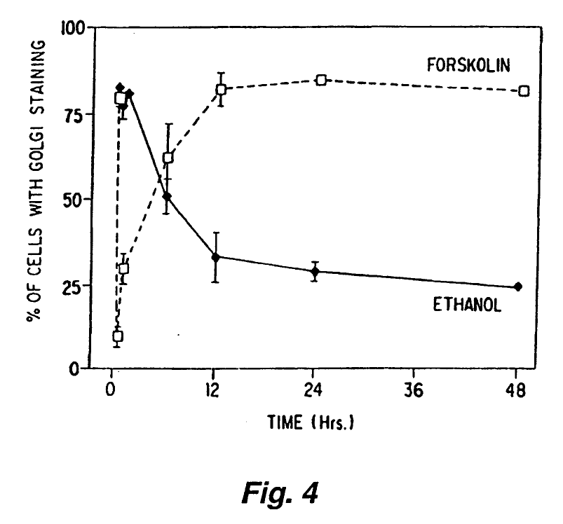

The time dependence of the localization of the Cα and the effects of other substances were investigated (FIG. 4) as follows: NG108-15 cells were cultured as described above. After two (2) days in defined media, cells received media containing either 200 mM ethanol or 1 μM forskolin for various lengths of time, as described below. Control cells received fresh media alone at the same time points. All slides were fixed four (4) days after plating and stained for Cα as described above. Similar experiments were performed using 25 mM and 50 mM ethanol over a period of four (4) to five (5) days; comparable results were obtained.

To obtain the numerical results, depicted in FIG. 4, fields were selected as described above, and cells were scored as either having Cα staining confined primarily to the Golgi or extensive staining outside of the Golgi. Data points are the mean+/−SEM of three (3) experiments.

The results will now be discussed with reference to FIG. 3 and FIG. 4. Panel A of FIG. 3 shows that the Cα was localized at the Golgi in the control cells, as before, and panel B of FIG. 3 shows that after forty eight (48) hours exposure to ethanol, Cα staining was found in the nucleus. This confirms the earlier described results.

Stimulation by 10 μM PGE, resulted in diffuse staining throughout the cell, as shown in panel C of FIG. 3. Similar results were achieved by treatment with 1 μM forskolin, as shown in panel D of FIG. 3. Maximal translocation of Cα away from the Golgi occurred after about thirty (30) minutes, with forskolin or PGE, which is when the micrographs shown in panels C and D of FIG. 3 were taken. This was followed by a return of staining to the Golgi for forskolin but not for ethanol (FIG. 4).

This is clearly contrasted with the effects of exposure to ethanol, where (see FIG. 4) after a relatively brief exposure to ethanol (from 30-60 minutes), little change in the localization of Cα was detected. After six (6) hours exposure to ethanol, translocation from the Golgi to the nucleus of the Cα was apparent, and after twelve (12) hours, most of the cells had developed prominent nuclear staining, with a corresponding decrease in Golgi staining. This staining remained through forty eight (48) hours of exposure to ethanol.

Thus, in screening for the effect of drugs on ethanol-induced altered long-term localization of Cα, best results will be obtained if the cells are left in the growth medium for at least about twelve (12) hours, and more preferably about forty eight (48) hours (two (2) days). The effects of ethanol exposure can be distinguished from the effects of other substances which produce a relatively temporary reversible change in Cα localization. Ethanol also causes an increased amount of Cα to be detected in the nucleus about 10 min. after exposure, which disappears by about 30 min.

The investigation of movement and activity of other PKA subunits particularly type I (RI) and type II (RII) regulatory subunits was investigated. Stains using monoclonal antibodies, prepared by conventional immunocytochemistry techniques, analogous to those described above for the Cα subunit were used. The RII subunit and the RI subunit were detected primarily on the Golgi apparatus. Ethanol was not found to have any significant effect on the localization of the RI subunit within the NG108-15 cell, but the RII subunit translocated to the nucleus.

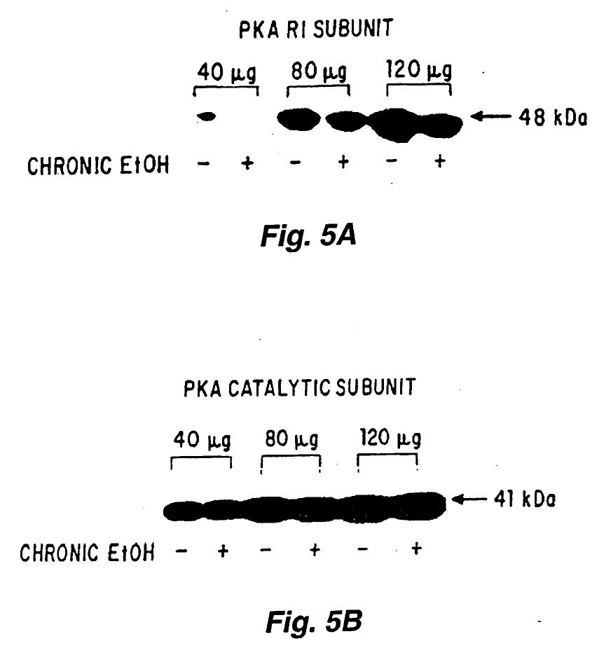

It was, however, found that the amount of RI subunit was decreased by exposure to ethanol. Results of a western blot analysis showing that exposure to 200 mM ethanol for forty eight (48) hours had no effect on the amount of Cα subunit, but produced a decrease of about 40% (43+/−3%) in the RI subunit are shown in FIG. 5. Thus, an alternative assay procedure can be provided by measuring the amount of PKA RI.

With the above results, the effects of ethanol on NG108-15 cells can thus be clearly identified. To produce a screen for therapeutic agents or drugs that modulate the cellular effects of exposure to ethanol, these cells can be exposed to ethanol in the presence of a drug whose activity is to be investigated, and the results compared to those obtained from cells exposed to ethanol in the absence of the drug.

### Example 2

### Ethanol Induced Translocation of δ-PKC and ε-PKC

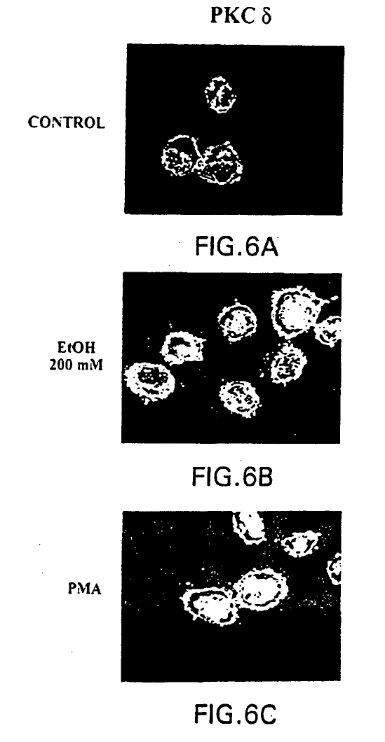

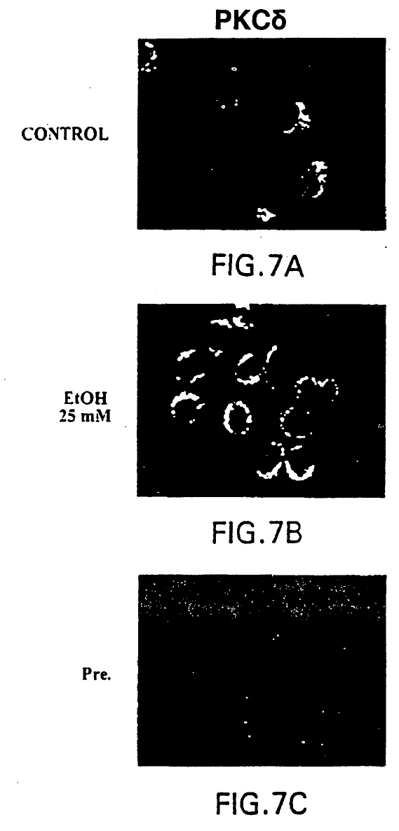

Immunohistochemistry of δ-PKC in NG108-15 cells, grown in defined medium, shows predominant Golgi staining (FIG. 6); approximately 70% of these cells show Golgi staining (Table 2). After 48 hours ethanol exposure (200 mM EtOH), δ-PKC is localized to the perinucleus and nucleus and absent from the Golgi (FIG. 6 and FIG. 7). More than 90% of the cells show perinuclear and nuclear staining (Table 2). The specificity of the fluorescence staining for δ-PKC is indicated by the absence of staining when the anti-5 antibody is preabsorbed with the immunizing peptide before labeling of the cells (FIG. 7). These results suggest that ethanol exposure causes translocation of δ-PKC from the Golgi to the perinucleus and nucleus since there is little δ-PKC remaining in the Golgi area (Table 2) after ethanol exposure.

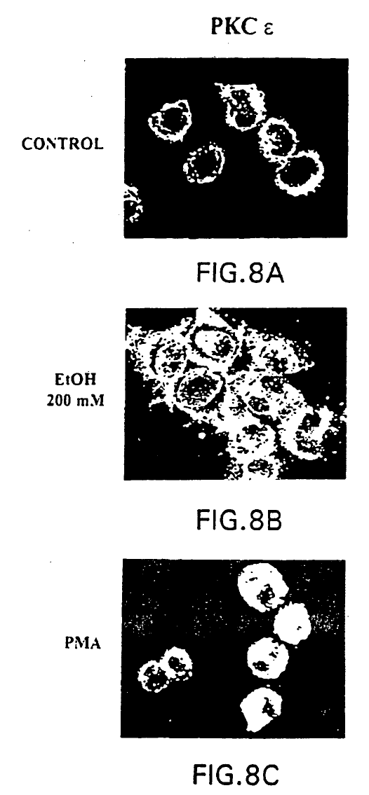

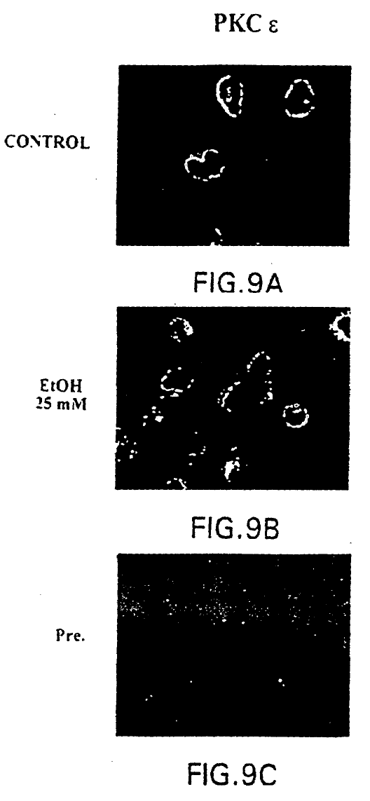

Ethanol also alters localization of ε-PKC. In naive cells, e —PKC is localized to the perinucleus in more than 90% of the cells (FIG. 8, FIG. 9 and Table 2), with no measurable cytoplasmic staining. After ethanol exposure, ε-PKC staining is observed throughout the cytoplasm in greater that 90% of the cells (FIG. 8, FIG. 9 and Table 2); perinuclear staining is still present in more that 90% of the cells (FIG. 8, FIG. 9 and Table 2). The staining for ε-PKC appears to be specific since no staining is observed when the anti-e antibody is preabsorbed with immunizing peptide (FIG. 9). Ethanol-induced altered localization of δ-PKC and ε-PKC is also observed after exposure to 25 mM ethanol for four (4) days (FIG. 7 and FIG. 9).

Ethanol-induced altered localization of PKC isozymes could be similar to that induced by phorbol esters or hormones or to sites different from these latter activators. Naive NG108-15 cells were, therefore, incubated in for ten (10) min. in 100 nM PMA, to then determine localization of δ-PKC and ε-PKC. On activation by PMA, the 8 isozyme is mainly translocated to the perinucleus (FIG. 6), suggesting that ethanol-induced translocation of δ-PKC is to sites similar to those occupied after activation by PMA. In contrast, translocation of ε-PKC due to PMA activation results in nuclear and perinuclear localization of this isozyme, different from ethanol-induced translocation to the cytoplasm (FIG. 8).

The effects of ethanol on NG108-15 cells can thus be clearly identified by determination of the localization of δ-PKC and ε-PKC. To produce a screen for therapeutic agents or drugs that modulate the cellular effects of exposure to ethanol, these cells can be exposed to ethanol in the presence of a drug whose activity is to be investigated, and the results compared to those obtained from cells exposed to ethanol in the absence of the drug.

TABLE 2

| δ-PKC (% cells) | | | | ε-PKC (% cells) | | | |
|---|---|---|---|---|---|---|---|
| Golgi Staining | | Perinuclear/nuclear staining | | Perinuclear staining | | Cytoplasmic staining | |
| Control | EtOH | Control | EtOH | Control | EtOH | Control | EtOH |
| 74 ± 7 | 2 ± 2 | 2 ± 2 | 93 ± 5 | 95 ± 2 | 94 ± 3 | 5 ± 2 | 94 ± 2 |
| (n = 5) | (n = 5) | (n = 5) | (n = 5) | (n = 5) | (n = 5) | (n = 5) | (n = 5) |

The percentage of particular immunostaining was obtained from five (5) experiments, each experiment was counted in four (4) fields per slide, three (3) or four (4) different slides in the same experience.

### Example 3

### Altered Localization of PKA Subunits in Lymphocytes and Neutrophils Exposed to Ethanol in Vitro

Non-alcoholic controls and chronic alcoholics completed an ICD10 questionnaire (a standard means for diagnosis of alcoholism) and were interviewed about their lifetime alcohol consumption. A blank questionnaire is provided in Table 3. Subjects who used other addictive drugs were excluded from the study. Blood alcohol levels were measured at the time of sampling using Alco-screen dipsticks. Routine CBC, triglyceride levels and liver function tests were carried out by the Clinical Hematology Laboratory at San Francisco General Hospital.

TABLE 3
Alcohol Dependence Questionnaire.

Have you experienced any of the following more than twice in the past year:
Compulsion

| | | | |
|---|---|---|---|
| 1) | Had a strong desire or urge to drink | Yes | No |
| 2) | Felt powerless over your drinking | Yes | No |
| 3) | Needed a drink so badly you couldn't think of anything else | Yes | No |

Impaired Control

| | | | |
|---|---|---|---|
| 4) | Tried to cut down or stop drinking and found you couldn't do it | Yes | No |
| 5) | Wanted to cut down or stop your drinking and found you couldn't do it | Yes | No |
| 6) | Ended up drinking much more than you intended to | Yes | No |
| 7) | Found it difficult to stop drinking once you had started | Yes | No |
| 8) | Kept on drinking for a longer period than you had intended to | Yes | No |
| 9) | Started drinking even though you hadn't intended to | Yes | No |

Withdrawal

| | | | |
|---|---|---|---|
| 10) | Been sick or vomited after drinking or the morning after | Yes | No |
| 11) | Felt depressed, irritable or nervous after drinking or the morning after | Yes | No |
| 12 | Found yourself sweating heavily or shaking after drinking or the morning | Yes | No |
| | after | | |
| 13) | Heard or seen things that weren't really there after drinking or the | Yes | No |
| | morning after | | |
| 14) | Taken a drink to keep yourself from shaking or feeling sick either after | Yes | No |
| | drinking or the morning after | | |

Tolerance

| | | | |
|---|---|---|---|
| 15) | Found that the same amount of alcohol had less of an effect than before | Yes | No |
| 16) | Found that you had to drink more than you once did to get the same effect | Yes | No |
| | Given up activities for drinking | | |
| 17) | Given up or cut down on activities or interests like sports or associations | Yes | No |
| | with friends, in order to drink | | |
| 18) | Lost ties with or drifted apart from a family member or friend because of | Yes | No |
| | your drinking | | |
| 19 | Had your chances for promotion, raises or better jobs hurt by your | Yes | No |
| | drinking | | |

Continued use despite harmful consequences

| | | | |
|---|---|---|---|
| 20) | Continued to drink alcohol even though it was a threat to your health | Yes | No |
| 21) | Kept drinking even though it caused you emotional problems | Yes | No |

Clinician: In order for a diagnosis of alcohol dependence (based on the ICD - 10) to be made, at least one item in each of three separate categories must be endorsed.

Isolation of Lymphocytes and Neutrophils

Blood (50 ml) from non-alcoholic controls and alcoholics were drawn into heparinized test tubes and cell types separated on a Ficoll Histopaque step density gradient (1.077/1.119 g/ml) according to English et al., J. Immunol. Methods 5:249-252 (1974). The tubes were centrifuged at 700× g for 30 min at room temperature. Two bands were collected; the upper band on top of the Ficoll contained platelets, monocytes and lymphocytes (lymphocyte fraction) and the lower band at the interface of the two Ficoll layers contained granulocytes and erythrocytes (neutrophil fraction). Contaminating erythrocytes were eliminated from the cells in the lower band by hypotonic shock. This treatment did not affect localization of PKA subunits. Cells in each band were diluted 1:10 in phosphate-buffered saline (PBS) and washed three times by centrifugation at 200× g. This step removed contaminating platelets from the lymphocyte fraction. 95% of the cells in each subpopulation were neutrophils or lymphocytes, respectively. Cell viability, determined by 0.4% Trypan Blue staining, was greater than 98% for both populations just after isolation and approximately 85% six hours later.

Cell Treatment.

Suspensions of lymphocytes (8×10⁵ cells/ml) or neutrophils (5×10⁵ cells/ml) from non-alcoholics were incubated in the absence or presence of various concentrations of ethanol for the indicated times. 150 μl of cell suspension was added to individual cytofunnels and the incubation stopped by centrifuging the cells in a Shandon II cytospin centrifuge for 3 min at 1000 rpm onto Falcon double chamber glass tissue culture slides in two stages. Neutrophils were spread at the bottom and lymphocytes on the top of the slides. The slides were then separated from the funnels and air dried before the cells were fixed with methanol at room temperature for 15 min.

Immunocytochemistry.

Blocking solution (1% normal goat serum, 0.1% Triton x-100 in PBS) was added to fixed cells and the slides incubated for two hours at room temperature. Buffer was then aspirated and cells were incubated overnight at 4° C. with primary antibody to either RIα, RIIα, RIβ, Cα, Cβ or Cγ (diluted 1:100 in PBS containing 0.2% fatty acid free bovine serum albumin (FAF-BSA)), and 0.1% Triton x-100. Slides were then washed 3 times with PBS, placed in humid chambers, and goat-anti-rabbit antibody conjugated to fluorescein isothiocyanate (diluted 1:250 in PBS containing FAF-BSA) for two hours at room temperature. After washing the slides 3 times in PBS, buffer was aspirated and one drop of Vectashield fluorescent mounting solution was added to each slide before covering and incubating overnight at 4° C.

Intracellular localization of PKA subunits was evaluated in 0.5 mm sections using a Bio-Rad 1024 con focal microscope (60× objective, total magnification 600×) and LaserSharp software. Images were further analyzed using NIH Image.

Localization in Neutrophils.

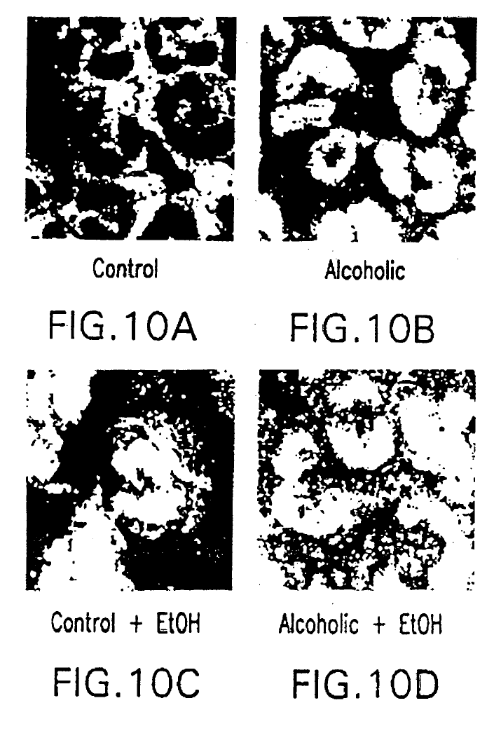

All three catalytic subunits of PKA (Cα, Cβ, Cγ) were present in the cytoplasm of neutrophils; addition of either 200 mM ethanol or 100 nM forskolin had no effect on localization of the catalytic subunits. RIα was also localized to the cytoplasm. However, after acute treatment with ethanol or forskolin, RIα translated from the cytoplasm to the nucleus. See, for example, FIG. 10A and FIG. 10C. After acute ethanol exposure, some RIα diffused out of the cells, most likely due to cellular membrane disruption as a result of the high sensitivity of neutrophils to in vitro treatments. This did not occur in control cells not receiving ethanol or forskolin. In contrast to RIα, ethanol and forskolin had no effect on the localization of RIIα. Ethanol or forskolin did not alter RIβ localization in neutrophils. No staining was observed for any antibodies after preincubation for two hours in the presence of a ten-fold excess of the respective immunogenic peptide prior to incubation with the leukocytes, indicating the specificity of staining.

Translocation of RIα to the nucleus in neutrophils was observed after 10 min of exposure to 200 mM ethanol. At lower, more physiological concentrations of ethanol, translocation of RIα occurred after 2 hours with 50 mM ethanol or after 4 hours with 25 mM ethanol.

Localization in Lymphocytes.

Ethanol exposure caused a dramatic translocation of Cα from the cytoplasm to the nucleus in lymphocytes. Translocation of Cα to the nucleus was also observed at low concentrations of ethanol (25 and 50 mM ethanol), beginning after two hours with 50 mM ethanol or after three hours of incubation with 25 mM ethanol. At lower concentrations of ethanol, a specific pattern of Cα staining in the nucleus can be seen, suggesting that localization in the nucleus is not due merely to diffusion but to binding to specific sites in the nucleus. Localization of Cβ, Cγ, and RIα in lymphocytes was not altered by ethanol. In human lymphocytes, we did, however, observe a partial translocation of the RIβ subunit from cytoplasm to nucleus. The RIIα subunit is localized in the nucleus of lymphocytes from non-alcoholic controls even without any treatment. Thus, in vitro ethanol exposure alters the localization of RIα and Cα in neutrophils and lymphocytes respectively.

### Example 4

### Altered Localization of PKA Subunits in Lymphocytes and Neutrophils from Actively Drinking Alcoholics

Lymphocytes and neutrophils were isolated from actively drinking alcoholics and were examined by immunocytochemistry, as described above.

RIα was localized in the nuclei of untreated neutrophils from alcoholics as shown in FIG. 10B. This is in agreement with the localization of PKA RIα in cells from non-alcoholic controls exposed to ethanol in vitro and suggests that, in neutrophils of alcoholics, PKA RIα is translated to the nucleus in vivo due to ethanol exposure. Further in vitro ethanol treatment of neutrophils from alcoholics did not result in any change in localization of RIα, as shown in FIG. 10D. RIIα and RIβ were localized to the cytoplasm in leukocytes from alcoholics either in the absence or presence of in vitro ethanol treatment, similar to results from non-alcoholic controls.

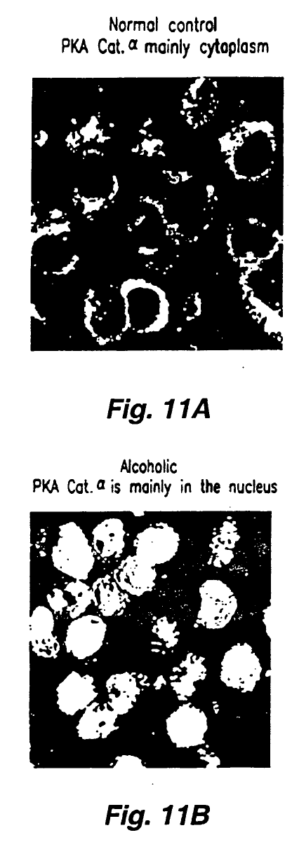

In vivo ethanol exposure also caused translocation of Cα. As shown in FIG. 11B, Cα was localized to the nucleus in untreated lymphocytes of alcoholics, similar to its localization in lymphocytes from non-alcoholic controls following acute in vitro ethanol exposure. Cα in lymphocytes from alcoholics remained in the nucleus after in vitro treatment with ethanol. Hence, there is a marked difference in the localization of RIα in neutrophils and Cα in lymphocytes between alcoholics and non-alcoholic controls.

Correlation Between Alcoholic State and PKA Subunit Localization

In order to determine whether there is a correlation between alcoholic state and PKA subunit localization, state, daily alcohol consumption, lifetime alcohol consumption, blood alcohol levels, total triglycerides and liver function profile of all participants in the study were obtained (Table 4). A correlation between alcoholic state and nuclear localization of RIα and Cα in neutrophils and lymphocytes, respectively, was found in seven out of eight alcoholics; in one alcoholic, nuclear localization of Cα and not RIα was correlated. One putative alcoholic showed no correlation, but he did not meet either of the ethanol consumption criteria. All non-alcoholic controls showed only cytoplasmic localization of RIα and Cα in both cell types. The correlation between nuclear localization of RIα and Cα and alcoholic state was statistically significant as evaluated by frequency distribution tests using Fisher's exact P value (P<0.0032 for neutrophils and P<0.0014 for lymphocytes.

TABLE 4
Correlation between alcoholic consumption and enzyme translocation

| | | | Alcohol | | Daily | | | | | Lifetime | | | Cα in |
|---|---|---|---|---|---|---|---|---|---|---|---|---|---|
| Patient | Alco | | consumption | | alcohol | | Liver | Triglyceride | | alcohol | | RIα in | nucleus |
| ID | screen | Alcoholic* | last 12 h | | consumption | | functions † | level | | consumption | | nucleus | ( ) # |
| 247 | − | ? | None | | 140 | ml | all | 160 | mg/dl | 597 | Kg | − | − |
| | | | | | | | normal | | | | | | |
| 351 | + | + | 240 | ml | 307 | ml | all | 560 | mg/dl | 1267 | Kg | + | + (2) |
| | | | | | | | abnormal | | | | | | |
| 255 | + | + | 235 | ml | 350 | ml | all | 172 | mg/dl | 969 | Kg | + | + (2) |
| | | | | | | | abnormal | | | | | | |
| 253 | + | + | 151 | ml | 276 | ml | all | 274 | mg/dl | 2106 | Kg | + | + |
| | | | | | | | normal | | | | | | |
| 259 | − | + | 52 | ml | 310 | ml | all | 71 | mg/dl | 1042 | Kg | + | + (3) |
| | | | | | | | normal | | | | | | |
| 267 | − | + | None | | 262 | ml | all | 65 | mg/dl | 752 | Kg | + | + |
| | | | | | | | normal | | | | | | |
| 269 | − | + | 60 | ml | 280 | ml | all | 339 | mg/dl | 2405 | Kg | + | + (2) |
| | | | | | | | normal | | | | | | |
| 271 | − | + | 149 | ml | 190 | ml | all | 73 | mg/dl | 2717 | Kg | + | + |
| | | | | | | | normal | | | | | | |
| 265 | − | + | None | | 80 | ml | all | 270 | mg/dl | 2189 | Kg | − | + |
| | | | | | | | normal | | | | | | |
| 312 | − | − | None | | 0 | ml | all | 140 | mg/dl | 81 | Kg | − | − (2) |
| | | | | | | | normal | | | | | | |
| 226 | − | − | None | | 26 | ml | all | 185 | mg/dl | 231 | Kg | − | − |
| | | | | | | | normal | | | | | | |
| 244 | − | − | None | | 18 | ml | all | 210 | mg/dl | 146 | kg | − | − (2) |
| | | | | | | | normal | | | | | | |
| 306 | − | − | None | | 0 | ml | all | 180 | mg/dl | 34 | Kg | − | − (4) |
| | | | | | | | normal | | | | | | |
| 144 | − | − | None | | 0 | ml | all | 120 | mg/dl | 28 | Kg | − | − |
| | | | | | | | normal | | | | | | |
| 206 | − | − | None | | 0 | ml | all | 200 | mg/dl | 14 | Kg | − | − |
| | | | | | | | normal | | | | | | |
| 310 | − | − | None | | 0 | ml | all | 131 | mg/dl | 10 | Kg | − | − (3) |
| | | | | | | | normal | | | | | | |

( ) = # of times blood drawn from subject

### Example 5

### Screening for Substances that Inhibit Ethanol-Induced Translocation of ε-PKC

NG 108-15 or CHO cells were pre-incubated in the presence or absence of the indicated inhibitor for 30 min. (except for GF which was pre-incubated for 1 hour), then further incubated in the absence or presence of 200 mM ethanol for 30 min.

TABLE 5
Inhibitors of Ethanol-Induced Translocation of ε-PKC.

| | | Predominant |
|---|---|---|
| | | ε-PKC |
| Compound | Class | Localization |
| None | | Perinucleus |
| Ethanol 200 mM | n/a | Cytoplasm |
| Ethanol + 15 ng/ml | Inhibitor of Gᵢ, Gₒ and G_{z} | Perinucleus |
| Pertussis toxin | mediated functions | |
| Ethanol + 2 × 10⁻⁴ | Adenylyl cyclase | Perinucleus |
| M MDL 12, 330A | inhibitor | |
| Hydrochloride | | |
| Ethanol + 20 μM Rₚ-cAMPS | PKA antagonist | Perinucleus |
| Ethanol + 10 μM Et-18-OCH₃ | PLC inhibitor | Perinucleus |
| Ethanol + 1 μM U 73122 | PLC inhibitor | Perinucleus |
| Ethanol + 100 nM MG_{F} | PKC inhibitor | Perinucleus |
| (Bisindoylmaleimide I) | | |
| Ethanol + 40 nM Gö | PKC inhibitor | Perinucleus |

### Example 6

### Screening for Substances that Alter Luciferase Expression in Transfected Cells Exposed to Ethanol

Luciferase is used as a reporter gene in a transient expression system to identify substances that alter the effects of ethanol on CRE-regulated gene expression. Cells are cultured as described in Dohrman et al., Proc Natl Acad Sci USA (1996) 93:10217-10221, and are transfected using Effectene (Qiagene) as described by the manufacturers. Twenty-four hours after transfection the cells are treated with 200 mM ethanol for 30 min. to 36 hr. Luciferase activity is measured using Luciferase Assay System (Promega) and normalized to β-galactosidase activity.

Such cells can be used as described above to screen substances for their ability to alter or mimic the cellular affects of ethanol.

Ethanol Dependent Luciferase Expression in NG108-15 Cells Transfected with GAL4-Luciferase Fusion Construct

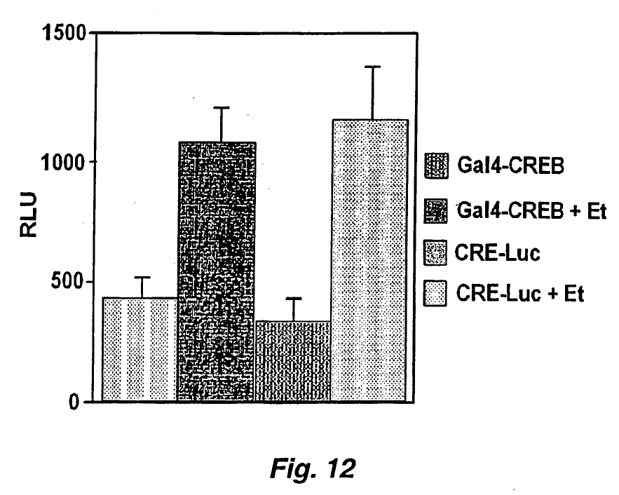

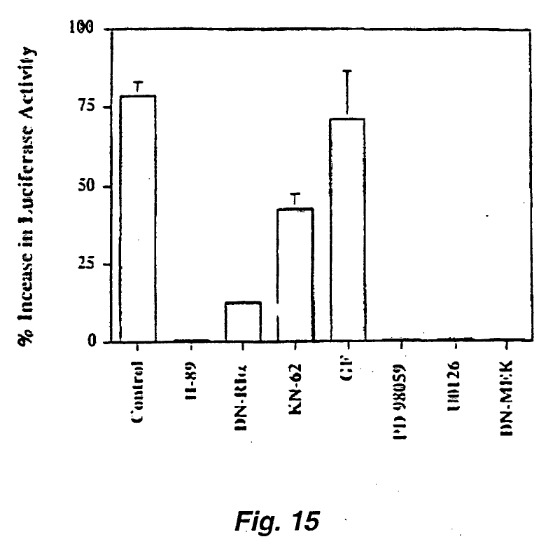

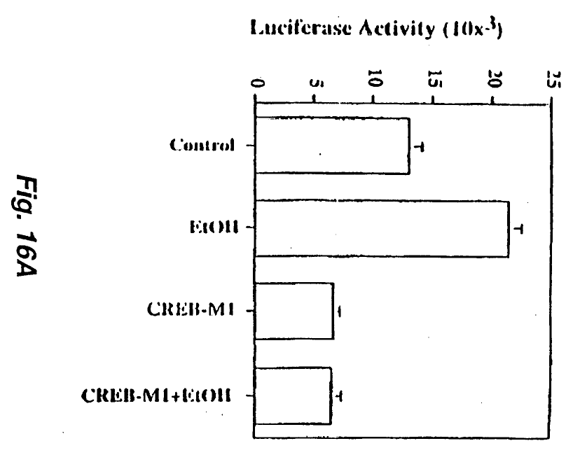

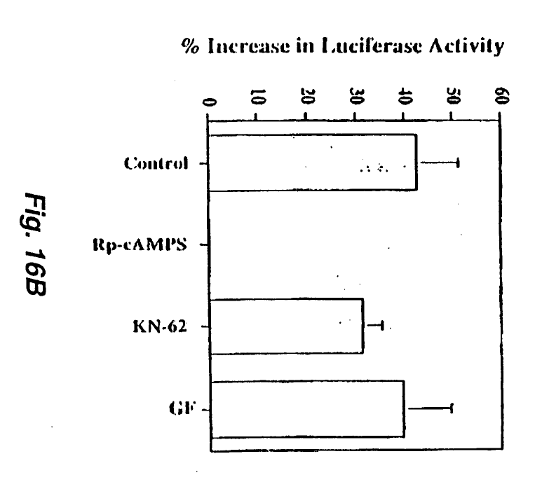

NG108-15 cells are co-transfected with a GAL4-CREB fusion construct and with a second construct containing the DNA binding site of GAL4 fused to luciferase as a reporter gene. As shown in FIG. 12, ethanol exposure increased the expression of luciferase approximately 2-fold. The ethanol-induced increase in luciferase activity induced was inhibited by Rp-cAMPS but not by the CaMK and PKC inhibitors KN-62 and GF, respectively (FIGS. 15 and 16B). These data demonstrate that phosphorylation and activation of CREB are required for ethanol-induced increases in CRE-mediated luciferase transcription.

Ethanol Dependent Luciferase Expression in NG108-15 Cells Transfected with CRE-Luciferase Fusion Construct

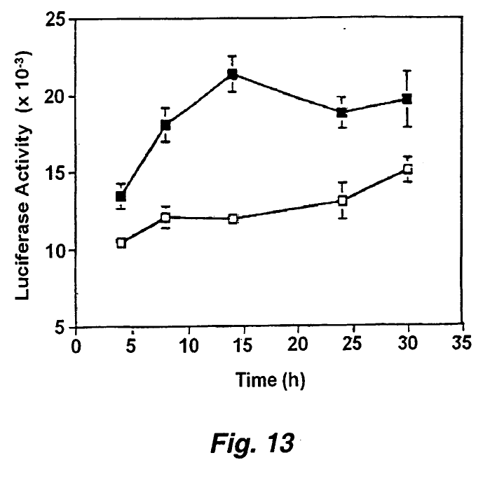

NG108-15 cells were co-transfected with a fusion construct containing the cAMP response element (CRE) fused to luciferase as a reporter gene (Stratagene). As shown in FIG. 12, ethanol exposure increased the expression of luciferase more than 2-fold. As shown in FIG. 13, an increase (29±6% above control) in luciferase activity was first apparent after a 4 hour exposure to 200 mM ethanol and a second increase (79±4% above control) occurred after 14 hours of exposure to 200 mM ethanol. A 14-hour exposure to 50 mM ethanol also resulted in an increase (about 25%) in luciferase activity.

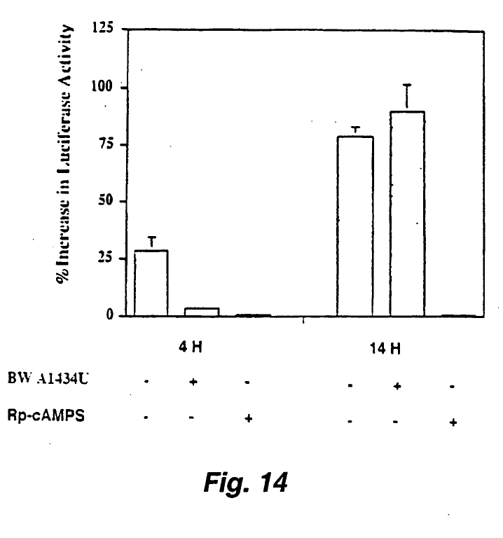

NG108-15 cells having the CRE-luciferase reporter gene were co-incubated with 200 mM ethanol and either an adenosine receptor antagonist (BW A1434U, 10 μM, Glaxo Wellcome) or a selective PKA inhibitor (Rp-cAMPS, 20 μM, Biolog Life Science Institute, La Jolla, Calif.). As shown in FIG. 14, the adenosine receptor antagonist blocked the peak in luciferase activity normally observed after 4 hours of ethanol exposure and the PKA inhibitor completely inhibited both the early and late phases of ethanol-induced increases in luciferase expression. The essential role of PKA in mediating ethanol-induced changes in luciferase expression was further demonstrated by the finding that H-89 (10 μM, Calbiochem, San Diego, Calif.), another selective PKA inhibitor, and DN-RIα (Clegg, C. H., Correll, L. A., Cadd, G. G., and McKnight, G. S. (1987) J Biol Chem 262(27), 13111-9)), a dominant negative form of PKA-RIα that has mutations at amino acid 200 in site A and amino acids 324 and 332 in site B and interferes with PKA activation, each inhibit the increase in luciferase expression that otherwise occurs after 14 hours of exposure to 200 mM ethanol (FIG. 15). CREB phosphorylation and activation are also essential for ethanol-induced increases in luciferase expression—a mutant form of CREB (CREB-M1, Dr. M. E. Greenberg) which, on account of the replacement of Ser-133 by alanine, can bind CRE but cannot be phosphorylated or activated, completely prevented ethanol-induced increases in luciferase activity at 14 hours (FIG. 16A). Since the ERK/MAPK pathway may regulate CREB phosphorylation and CRE-mediated gene expression, NG108-15 cells having the luciferase construct were co-incubated with 200 mM ethanol and a MEK inhibitor (PD98059 (2 nM, Calbiochem, San Diego, Calif.) or U0126 (1 μM, Calbiochem, San Diego, Calif.)) or a dominant negative MEK construct (DN-MEK, Dr. R. Seger) in which Lys-97 was replaced by alanine. As shown in FIG. 15, the MEK inhibitors and the dominant negative MEK construct all eliminated the increase in luciferase activity normally seen after 14 hours of ethanol exposure. Thus, long term changes in ethanol-induced, CRE-regulated gene expression are dependent upon ERK/MAPK signaling.

Ethanol Dependent Luciferase Expression in CHO Cells Transfected with CRE-Luciferase Fusion Construct

CHO cells are transiently transfected with Gal4-CREB and Gal4-Luciferase or with CRE-Luciferase (Stratagene) utilizing Effectene (Qiagene) as described the manufacturers. Twenty-four hours after transfection the cells are treated with 200 mM ethanol for 30 min. to 36 hr. Luciferase activity is then measured using Luciferase Assay System (Promega) and normalized to β-galactosidase activity.

Drug Dependent Luciferase Expression in NG108-15 Cells Transfected with CRE-Luciferase Fusion Construct

NG108-15 cells were co-transfected with a fusion construct containing the cAMP response element (CRE) fused to luciferase as a reporter gene (Stratagene). Incubation of these cells for four hours with an opioid agonist (10 nM enkephalin (D-ala2,D-leu5) (“DADLE”)) caused a forty percent (40%) increase in luciferase expression that closely resembled the ethanol-induced changes after four hours.

### Example 7

### Effects of Various Receptor Agonists and Antagonists on δ-PKC, δ-PKC or PKA Cα Translocation

To determine whether addictive drugs other than ethanol would cause translocation of some proteins, we examine the effects of various receptor agonists and antagonists on ε-PKC δ-PKC or PKA Cα translocation in NG108-15 and CHO cells expressing the applicable receptor. As shown below in Table 6, δ-opioid receptor agonists, cannabinoid receptor agonists, and dopamine receptor agonists do cause translocation in such NG108-15 cells, but muscarinic agonists and α2_{B} adrenergic receptor agonists do not, suggesting that addictive drugs other than ethanol also cause translocation. Pre-treatment with 10 μM spiperone, a dopamine receptor antagonist, blocked NPA-induced translocation of δ-PKC, ε-PKC and PKA Cα and had no effect upon ethanol-induced translocation of δ-PKC, ε-PKC and PKA Cα. Similar translocation of δ-PKC, ε-PKC and PKA Cα was observed in dopamine D2 receptor-expressing CHO cells after treatment with ethanol, dopamine receptor agonist NPA and/or dopamine receptor antagonist spiperone. Adenosine is also implicated in some behavioral effects of ethanol. Interestingly, adenosine receptor agonists cause translocation of ε-PKC and adenosine receptor antagonists prevent ethanol-induced translocation of PKA-Cα and ε-PKC but have no effect on ethanol-induced translocation of δ-PKC.

TABLE 6
Translocation of ε-PKC, δ-PKC, or PKA Cα by Gᵢ- or Gₛ-coupled receptors.

| | | Predominant |
|---|---|---|
| Treatment | Protein | Localization |
| None | ε-PKC | Perinucleus |
| 200 mM Ethanol | ε-PKC | Cytoplasm |
| 50 nM D2 Dopamine receptor agonist NPA | ε-PKC | Cytoplasm |
| 1 μM δ-Opiate receptor agonist D-ala-D-leu-enkephalin | ε-PKC | Cytoplasm |
| 2 μM Cannabinoid receptor agonist R(+) methanindamide | ε-PKC | Cytoplasm |
| 20 μM Muscarinic acetylcholine receptor agonist (Carbachol) | ε-PKC | Perinucleus |
| 10⁻⁵ M α2_{B} Adrenergic receptor agonist (UK14304) | ε-PKC | Perinucleus |
| 20 nM Adenosine A1 receptor agonist CCPA (2-chloro-N⁶- | ε-PKC | Cytoplasm |
| cyclopentyladenosine) | | |
| 100 nM Adenosine A2 receptor agonist CGS-21680 HCl | ε-PKC | Cytoplasm |
| 10 μM Dopamine receptor antagonist spiperone + 50 nM D2 | ε-PKC | Perinucleus |
| Dopamine receptor agonist NPA | | |
| 10 μM Dopamine receptor antagonist spiperone + 200 mM | ε-PKC | Cytoplasm |
| ethanol | | |
| 10 μM Adenosine receptor antagonist BW 1434 + 200 mM | ε-PKC | Perinucleus |
| ethanol | | |
| None | δ-PKC | Golgi |
| 200 mM Ethanol | δ-PKC | Perinucleus & |
| | | Nucleus |
| 50 nM D2 Dopamine receptor agonist NPA | δ-PKC | Perinucleus & |
| | | Nucleus |
| 10 μM Dopamine receptor antagonist spiperone + 50 nM D2 | δ-PKC | Golgi |
| Dopamine receptor agonist NPA | | |
| 10 μM Dopamine receptor antagonist spiperone + 200 mM | δ-PKC | Perinucleus & |
| ethanol | | Nucleus |
| 10 μM Adenosine receptor antagonist BW 1434 + 200 mM | δ-PKC | Perinucleus & |
| ethanol | | Nucleus |
| None | PKC Cα | Golgi |
| 200 mM Ethanol | PKC Cα | Nucleus & |
| | | Cytoplasm |
| 50 nM D2 Dopamine receptor agonist NPA | PKA Cα | Nucleus & |
| | | Cytoplasm |
| 10 μM Dopamine receptor antagonist spiperone + 50 nM D2 | PKA Cα | Golgi |
| Dopamine receptor agonist NPA | | |
| 10 μM Dopamine receptor antagonist spiperone + 200 mM | PKA Cα | Nucleus & |
| ethanol | | Cytoplasm |
| 10 μM Adenosine receptor antagonist BW 1434 + 200 mM | PKA Cα | Golgi |
| ethanol (10 min exposure) | | |
| 10 μM Adenosine receptor antagonist BW 1434 + 200 mM | PKA Cα | Nucleus |
| ethanol (48 hr exposure) | | |

### Example 8

### Mechanisms of Ethanol- and Dopamine-Mediated Translocation of PKC

The mechanisms underlying ethanol- and dopamine-mediated translocation of ε-PKC and δ-PKC were studied by altering the activity of various molecules that might be in the pathways leading to translocation.

When phospholipase C (PLC) activity was inhibited by the administration of pertussis toxin, both ethanol- and NPA-induced translocation of ε-PKC, δ-PKC and PKA Cα were inhibited in CHO and NG108-15 cells expressing the dopamine D2 receptor. Similarly, PLC inhibitors U-73122 and Et-18-OCH3 inhibited ethanol-induced translocation of ε-PKC by 87% in such CHO cells and by 84% and 96%, respectively, in such NG108-15 cells. This suggests that ethanol and dopamine cause translocation of ε-PKC and δ-PKC by activating PLC, which increases diacylglycerol levels.

Administration of PKA antagonist Rp-cAMPS blocked both ethanol- and NPA-induced translocation of ε PKC in CHO and NG 108-12 cells expressing the dopamine D2 receptor but did not affect ethanol- or NPA-induced δ PKC translocation in such cells. Activation of PKA directly by administration of PKA agonist Sp-cAMPS or indirectly by using forskolin or PGE1 to increase cAMP levels caused ε PKC to translocate to the cytoplasm, similar to ethanol- or NPA-induced translocation, but did not induce translocation of δ PKC. Thus, PKA activity is both necessary and sufficient to enable ethanol- and NPA-induced translocation of ε PKC to the cytoplasm but does not appear to have a role in ethanol- or NPA-induced translocation of δ PKC.

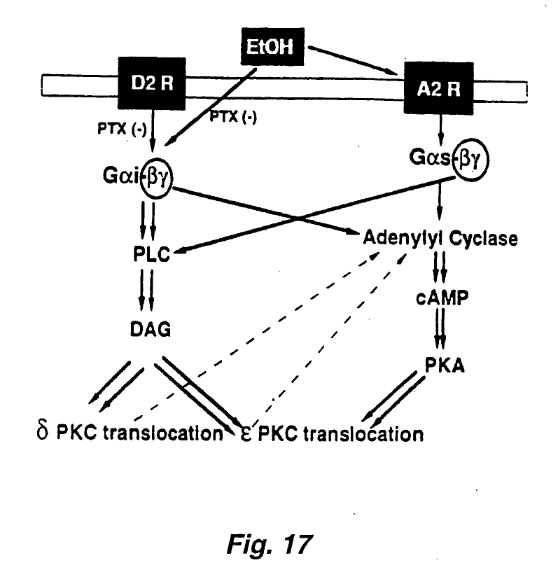

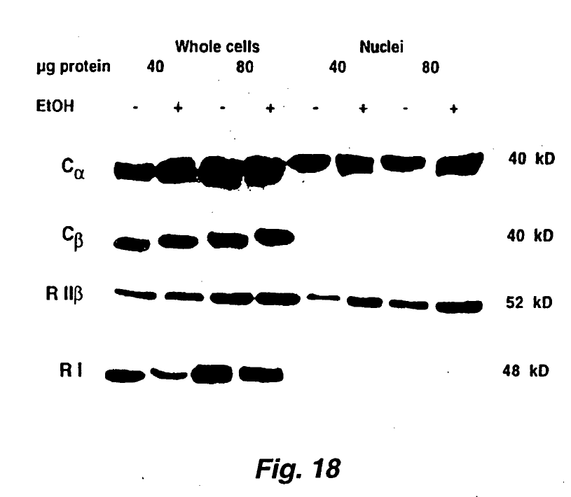

Without limitation to a particular theory, the inventors believe that the model shown in FIG. 17 explains the results presented in this Example 8 and the preceding Example 7. According r to this model, dopamine/NPA induces PKA activation by releasing βγ, which activates adenylyl cyclase, and ethanol induces PKA activation by stimulating adenosine A2 receptors and by releasing βγ from Gᵢ/Gₒ, which activate adenylyl cyclase. Thus, the ethanol- and dopamine-triggered pathways appear to intersect at adenylyl cyclase, which accounts for their shared translocation of 0PKA-Cα. These pathways also appear to Intersect at PLC, which accounts for their shared, PKA-independent, translocation of δ PKC. The translocation of ε PKC as a result of NPA or ethanol exposure appears to be due to activation of PLC and PKA.

The inventors believe that δ-opioids and cannabinoids activate their respective receptors which in turn activate the same pathways that interact with ethanol-induced translocation of PKA-Cα, δ PKC and ε PKC in a manner that is analogous to the activation of the dopamine receptor described above.

### Example 9

### Synergistic Interactions of Ethanol and Other Addictive Drugs

The common signaling pathways of ethanol and dopamine D2 receptors demonstrated in Examples 7 and 8 was further explored in dosage studies. Although 30 minute incubations of dopamine D2-receptor-expressing NG108-15 and CHO cells with either 25 mM ethanol or 5×10¹¹⁰ M NPA did not cause any perceptible translocation of PKA-Cα, 30-minute co-incubation of these concentrations of ethanol and NPA caused maximal translocation of PKA-Cα. Translocation of δ PKC and δ PKC in such NG108-15 and CHO cells was induced by co-incubation with 10⁻⁹ M NPA and 25 mM ethanol, but neither agent alone caused translocation at these concentrations. When levels of CREB phosphorylation were measured using an antibody specific for phosphorylated CREB, co-incubation of 25 mM ethanol and 1 nM NPA for 10-180 minutes also showed synergistic interactions. These results demonstrate that ethanol and dopamine act synergistically with respect to translocation of PKA and PKC and CREB phosphorylation. They also suggest that cells that express the dopamine D2 receptor are much more sensitive to low levels of ethanol than cells that do not express this receptor.

Synergistic translocation of PKA-Cα was also demonstrated after 10 minute co-incubations of: δ-opioid (10⁻¹¹M DADLE) and ethanol (25 mM); cannabinoid (2×10⁻¹⁰M methanindamide) and ethanol (25 mM); δ-opioid (10⁻¹¹M DADLE) and dopamine (10⁻¹⁰M NPA); and cannabinoid (2×10⁻¹⁰M methanindamide) and dopamine (10⁻¹⁰M NPA). However, a 10 minute coincubation of δ-opioid (10⁻¹¹M DADLE) and cannabinoid (2×10⁻¹⁰M methanindamide) did not result in synergistic translocation of PKA-Cα.

### Example 10

### Imaging of GFP-tagged Fusion Proteins in Cells

The Green Fluorescent Protein (GFP) is employed as a tag to facilitate the imaging of certain proteins in mammalian tissue cell culture. For the current experiments we have constructed the GFP-tagged fusion proteins GFP-Cα and GFP-RIIβ. The fusion proteins PKCδ-GFP and PKCε-GFP were obtained from Christer Larsson, Lund University, Sweden. The methodology of the experiments is to introduce the DNA encoding these proteins into the cell of interest and utilize the cell's capacity to synthesize the foreign protein to image the movement of the protein under controlled conditions. Briefly, cDNA encoding the protein of interest were cloned into a pGFP vector (available from Clontech Laboratories, Inc.) under the control of the immediate early promoter of human cytomegalovirus (CMV). NG108-15 cells are cultured in a defined medium as described above, split onto plastic chamber slides, and allowed to establish for a further 24 hours. The cells are then transfected with 400 ng of supercoiled plasmid in separate transfection experiments. The transfection is mediated by the commercially available Qiagen Effectene™ system. The efficiency of DNA transfer is about 30% of NG108-15 cells. These cells are allowed to express the protein for 20 hours prior to stimulation with the drug of interest. This reaction is terminated by washing and fixing the cells with 4% Paraformaldehyde.

Primary cell cultures of rat hippocampus include bipolar include bipolar and pyramidal neurons and glial cells. These cells are transfected with the same DNA concentration as above and with the same reagents, but the medium is maintained before and after transfection as these cells condition their own medium.

Imaging of the proteins is carried out on a BioRad laser scanning confocal microscope and a Leica upright microscope. These images are processed in Adobe Photoshop™ software and Spot™ software respectively.

The fusion constructs were made by linking, in frame, a polynucleotide encoding a 20 Kd GFP and a polynucleotide encoding the protein of interest. Table 7 describes the primary structure of the resultant GFP-fusion proteins.

TABLE 7
GFP Fusion Proteins.

| Fusion | N-terminus | Spacer | C-terminus |
|---|---|---|---|
| GFP-PKA Cα | 20 kd GFP | his tag | ~40 kd PKA Cα |
| | (aa 1-238) | (20 aa) | (aa 1-351) |
| GFP-PKA RIIβ | 20 kd GFP | — | ~52 kd PKA RIIβ |
| | (aa 1-238) | | (aa 1-418) |
| δPKC-GFP | ~76 Kd δPKC | — | 20 kd GFP |
| | (aa 1-676) | | (aa 1-238) |
| εPKC-GFP | ~94 Kd εPKC | — | 20 kd GFP |
| | (aa 1-737) | | (aa 1-238) |

Tables 8-10 indicate the translocation of several GFP fusion proteins. GFP-Cα translocated to the nucleus in ethanol exposed cells and cells exposed to NPA, as does PKA Cα. PKCδ-GFP translocated from the Golgi to the perinucleus, as does δ-PKC. GFP-RIIβ did not exhibit the translocation pattern observed for δ-PKC or RIIβ. This is likely due to overly high levels of expression, which may have saturated other cellular factors involved in the transaction. Lowering the expression levels should lead to the expected translocation pattern.

TABLE 8
Subcellular localization of GFP-Cα.

| | Control | +100 mM | 50 nM |
|---|---|---|---|
| Cell Line | Unstimulated | EtOH/30 min | NPA/30 min |
| NG108-15/D2R | Golgi Apparatus | Nucleus | Nucleus |
| | | [>50% cells] | [>50% cells] |
| 1° Neurons | Golgi Apparatus | Nucleus | ND* |
| | | [>50% cells] | |
| 1° Glia | Golgi Apparatus | Nucleus | Nucleus |
| | | [<20% cells] | [>50% cells] |

TABLE 9
Subcellular Localization of PKCδ-GFP.

| | Control | +100 mM | 50 nM |
|---|---|---|---|
| Cell Line | Unstimulated | EtOH/30 min | NPA/30 min |
| NG108-15/D2R | Golgi Apparatus | Perinucleus/ | ND |
| | | membranes | |
| | | [>50% cells] | |
| 1° Neurons | Golgi Apparatus | ND | ND |
| 1° Glia | Golgi Apparatus | Golgi Apparatus | ND |

TABLE 10
Subcellular Localization of GFP-RIIβ.

| | Control/ | +100 mM | 50 nM |
|---|---|---|---|
| Cell Line | Unstimulated | EtOH 30 min | NPA 30 min |
| NG108-15/D2R | Golgi Apparatus | Golgi Apparatus | ND |
| 1° Neurons | Golgi Apparatus | Golgi Apparatus | ND |
| 1° Glia | Golgi Apparatus | Golgi Apparatus | ND |

* = Not Determined

### Example 11

### Nuclear Localization of PKA RIIβ in Cells Exposed to Ethanol

NG-108 cells were exposed to 200 mM ethanol for 48 hours. Whole cells homogenates and homogenates from isolated nuclei were subjected to SDS-PAGE. Western analysis was performed with antibodies which recognize PKA Cα, PKA CB, PKA RIIβ, and PKA RI. As shown in FIG. 13, PKA Cα and PKA RIIβ translocated to the nucleus and PKA CB and PKA RI did not translocate.

### Example 12

### Synergy of Dopamine D2 and Adenosine A2 Receptors Activates PKA Signaling Via βγ Dimers: βγ Regulation of Sustained Ethanol Consumption

### Summary

Dopamine release is activated by ethanol and addicting drugs, but molecular mechanisms linking dopaminergic signaling to neuronal responses and drinking behavior are poorly understood. We report that dopamine-D2 receptors induce PKA Cα translocation and increase CRE-regulated gene expression. Ethanol also activates PKA signaling. Sub-threshold concentrations of the D2 agonist NPA and ethanol, without effect alone, together cause synergistic PKA translocation and CRE-mediated gene transcription. D2 and adenosine receptor blockade, pertussis toxin, RpcAMPS or overexpression of dominant-negative peptides which sequester βγ dimers prevents synergy. Importantly, overexpression of a βγ scavenger peptide in the nucleus accumbens strikingly reduces sustained ethanol consumption. We propose that synergy of D2 and A2 confers ethanol hypersensitivity and that βγ dimers are required for ethanol drinking behavior.

### Introduction.

Dopaminergic signaling in the nucleus accumbens (NAc) is activated by all addicting drugs (Robbins and Everitt (1999) Nature 398:567-570; Wise and Bozarth (1987) Psychol. Rev. 94: 469-492). Release of dopamine occurs in anticipation of drinking or during exposure to ethanol and is thought to contribute to incentive and reinforcement of ethanol consumption (Hodge et al. (1997) Alcohol Clin. Exp. Res. 21: 1083-1091; Imperato and Di Chiara (1986) J. Pharmacol. Exp. Ther. 239: 219-228; Weiss et al. (1993) J. Pharmacol. Exp. Ther. 267: 250-258). Furthermore, ethanol preference and sensitivity are markedly reduced in mice lacking D2 receptors (Phillips et al. (1998) Nature Neurosci. 1:610-615). In contrast, overexpression of D2′ receptors (D2) has also been reported to reduce ethanol preference and intake (Thanos et al. (2001) J. Neurochem. 78: 1094-1103). D2 activation inhibits adenylyl cyclase via the GTP-binding protein Gi in many tissues, cell lines, and brain (Obadiah et al. (1999) Cell Mol. Neurobiol. 19: 653-664). D2 activation also produces other intracellular events (Lledo et al. (1992) Neuron 8:455-463; Waszczak et al. (1998) Proc. Natl. Acad. Sci., USA, 95: 11440-11444; Vallar and Meldolesi (1989) Trends Pharmacol. Sci. 10: 74-77; Piomelli et al. (1991) Nature 353: 164-167; Gordon et al. (2001) Mol. Pharm. 58: 153-160). Despite the importance of dopamine in responding to ethanol, the molecular mechanism linking D2 signaling to neuronal activation and drinking behavior is not clear.

We have developed an NG108-15/D2 cell line expressing D2 (Asai et al. (1998). Alcohol Clin. Exp. Res. 22: 163-166) to study D2 responses to ethanol. We previously used wild type NG108-15 cells to demonstrate short term ethanol-induced increases in cAMP (Gordon et al. (1986) Proc. Natl. Acad. Sci., USA, 83: 2105-2108), PKA translocation and CRE-mediated gene expression. These changes occur via ethanol-mediated increases in extracellular adenosine and consequent adenosine A2 receptor (A2) activation. Because there is evidence in the literature that D2 activation can increase cAMP (Watts and Neve (1996) Mol. Pharmacol. 50: 966-976), we asked whether D2 can activate the PKA pathway. We find the D2 agonist NPA increases cAMP, causes translocation of the catalytic subunit of PKA (Cα) and increases CRE-mediated gene expression. Moreover, we have discovered that (a) subthreshold concentrations of NPA and ethanol synergistically induce PKA Cα translocation and increase CRE-mediated gene expression, (b) free βγ subunits released from G-proteins mediate these synergistic responses; (c) dopamine and adenosine receptor activation is required for synergy; and (d) overexpression of a dominant negative βγ scavenger peptide, which sequesters free βγ dimers in the NAc strikingly reduces ethanol consumption by rats.

### Results

D2 Activation Induces PKA Translocation.

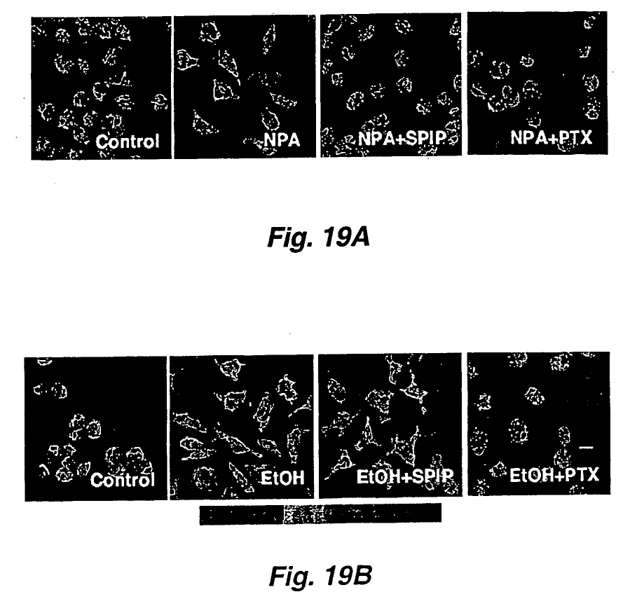

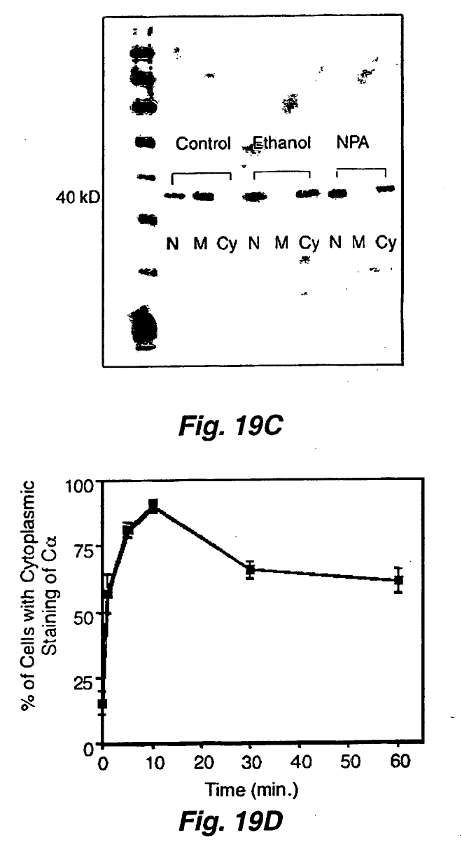

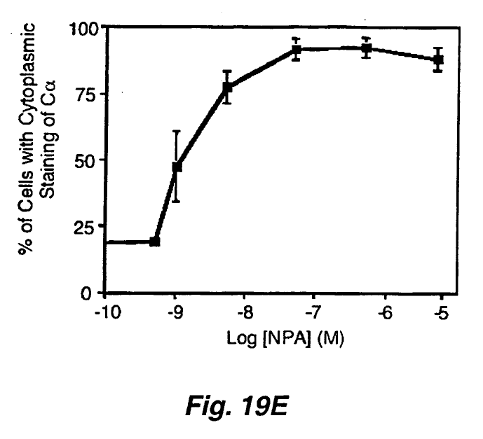

NG108-15 cells that express D2 (NG108-15/D2) were incubated for 10 min with the D2 agonist NPA; anti-PKA Cα antibodies were used to follow PKA Cα localization. FIGS. 19A and 19B show that PKA Cα is localized primarily to the Golgi area in untreated cells. D2 activation by 50 nM NPA causes translocation of PKA Cα from the Golgi into the cytoplasm and nucleus in 10 minutes (FIG. 19A). Ethanol (100 mM) mimics NPA-induced translocation of PKA-(FIG. 19B). Western blot analyses of subcellular fractions shows that NPA, or ethanol, each cause translocation of PKA Cα from the membrane fraction to the nucleus and cytoplasm after a 10-min incubation (FIG. 19C).

Specificity of the NPA response is indicated in FIG. 19A. NPA-induced translocation of PICA Cα is blocked by spiperone, a D2 antagonist (FIG. 19A). Spiperone has no effect on ethanol induced PKA Cα; translocation. (FIG. 19B). NPA-induced PKA Cα translocation occurs within minutes, reaching a maximum at 10 min (FIG. 19D); the Km is 1.6± 0.5×10⁻⁹ M (FIG. 1E).

NPA-Induced Translocation of PKA Cα is Mediated Via Increases in cAMP.

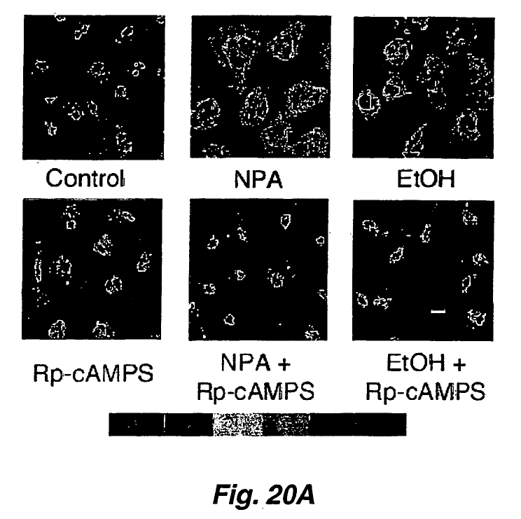

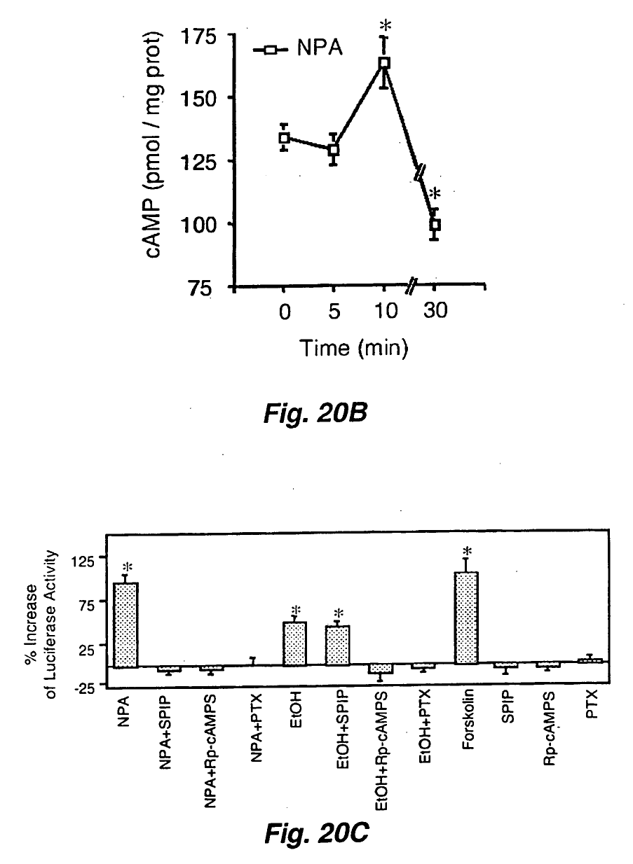

When cyclic AMP binds to the regulatory subunit of PKA, Cα is released from the holoenzyme and translocates to different subcellular compartments. Ethanol increases cAMP production (Nagy et al. (1989) Mol. Pharm. 36: 744-748; Sapru et al. (1994) J. Pharmacol. Exp. Ther. 271: 542-548) and causes translocation of PKA Cα to the cytoplasm and nucleus (FIG. 19B). Rp-cAMPS, which prevents binding of cAMP, blocks ethanol-induced translocation of Cα (FIG. 20A). Rp-cAMPS also blocks NPA-induced translocation (FIG. 20A). This suggested that NPA might increase cAMP production. FIG. 20B shows that NPA increases cAMP levels at 10 minutes; decreased levels are observed at 30 min.

NPA Induces CRE-Mediated Gene Expression.

Ethanol-induced translocation of PKA into the nucleus in NG108-15 cells leads to CREB phosphorylation (Constantinescu et al. (1999) J. Biol. Chem. 274: 26985-26991) and activation of CRE-mediated gene expression. We determined whether NPA also increases CRE-mediated gene expression. Cells were transiently transfected with a CRE-luciferase reporter construct to measure gene expression via endogenous CRE-binding protein (CREB). Transfected cells were exposed to 50 nM NPA and other agents for 10 min and assayed for luciferase activity 5 hours later (FIG. 20C). NPA increases luciferase activity to the same extent as forskolin. Spiperone blocks NPA-dependent increases in luciferase activity but has no effect on ethanol-induced luciferase expression; both are blocked by Rp-cAMPS. Spiperone and Rp-cAMPS alone were without effect. These data suggest that D2-mediated increases in cAMP and PKA Cα translocation to the nucleus activates CRE-mediated gene expression.

Synergy Between NPA and Ethanol-Induced Translocation of PKA Cα and CRE-Mediated Gene Expression

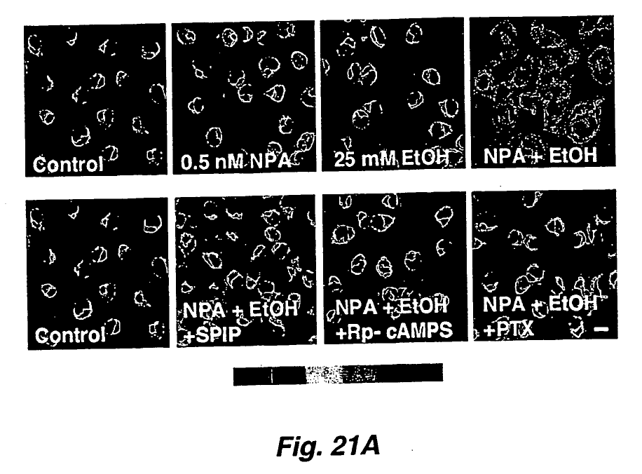

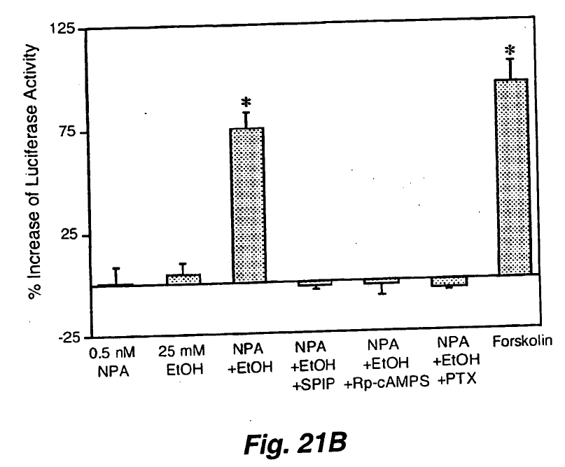

Since NPA or ethanol activates the PKA pathway from cAMP through CRE-mediated increases in gene expression, we searched for synergy between NPA and ethanol. NPA (0.5 nM) or ethanol (25 mM) alone did not cause PKA translocation (FIG. 21A). However, these subthreshold concentrations of NPA and ethanol together produce a striking synergistic increase in PKA C□translocation and CRE-mediated luciferase activity (FIGS. 21A, 21B). Most importantly, the synergistic increase in gene expression is as great as that produced by forskolin (FIG. 21B). Synergy depends on D2 activation and increased cAMP, because it is blocked by the D2 antagonist spiperone and Rp-cAMPS (FIGS. 21A and 21B).

NPA and Ethanol-Induced Synergy; PKA Cα Translocation and Gene Expression is Due to Activation of Adenylyl Cyclase by βγ Dimers Released from Gi/o

D2 couples to the Gi/o family of trimeric GTP-binding proteins (Watts and Neve (1996) Mol. Pharmacol. 50: 966-976). Gi/o activation is blocked by pertussis toxin (PTX). Preincubation with PTX prevents NPA-induced PKA Cα translocation and CRE-mediated gene expression (FIGS. 19A and 20C). PTX also blocks Cα translocation and gene expression induced by 100 mM ethanol (FIGS. 19B and 20C). PTX alone had no effect on cell morphology, PKA Cα localization (data not shown), or gene expression (FIG. 20C). PTX also inhibits synergy of PKA Cα translocation (FIG. 21A) and CRE-mediated gene expression (FIG. 21B). These results suggest that Gi/o is required for NPA and ethanol-induced Cα translocation, gene expression and synergy. βγ dimers derived from Gi/o activate adenylyl cyclase (AC) isozymes II and IV (Baker et al. (1999) J. Neurosci. 19: 180-192; Federman et al. (1992) Nature 356: 159-161; Inglese et al. (1994). Proc. Natl. Acad. Sci. USA, 91: 3637-3641; Tang and Gilman (1991) Science 254: 1500-1503). NG108-15/D2 cells express AC II and TV (FIG. 4A). Because PTX prevents release of βγ dimers from Gi/o (Watts and Neve (1997) Mol. Pharm. 52: 181-186), we studied the role of βγ dimers in PKA Cα translocation, gene expression and synergy.

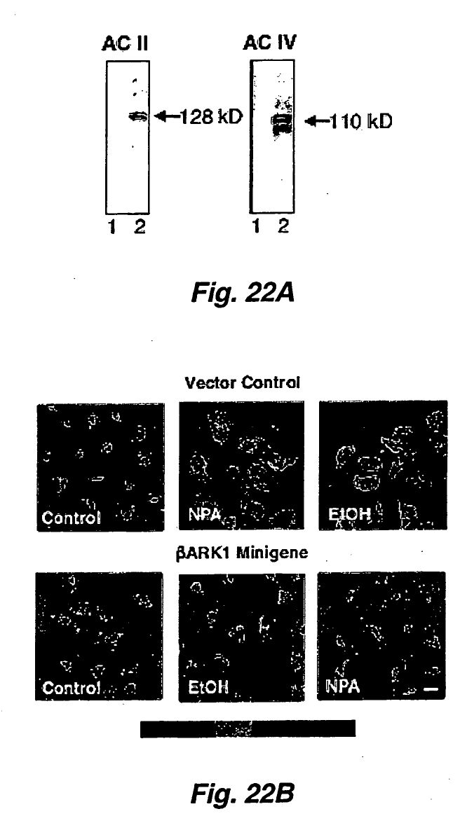

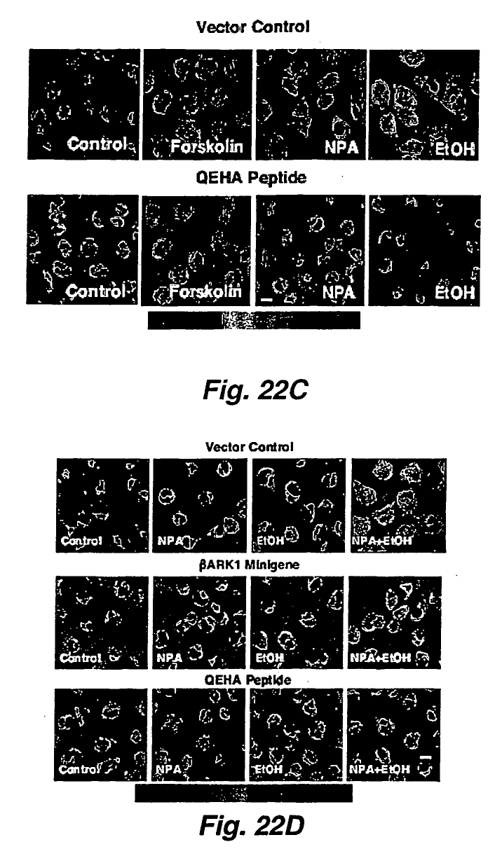

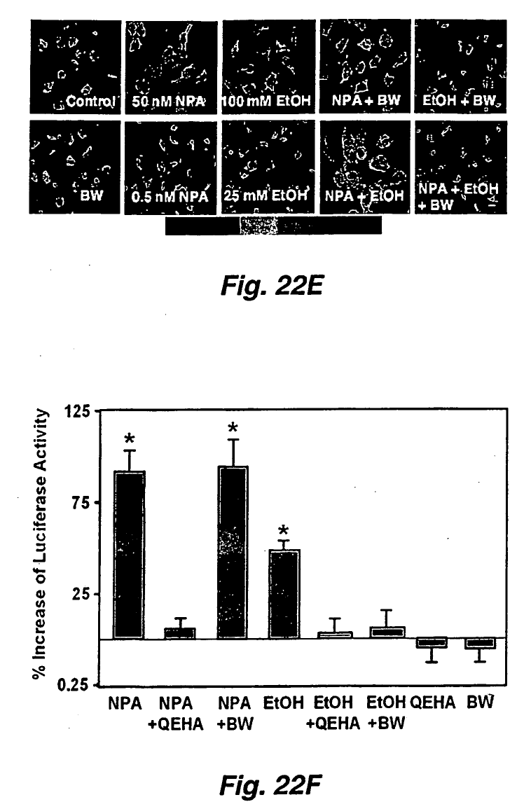

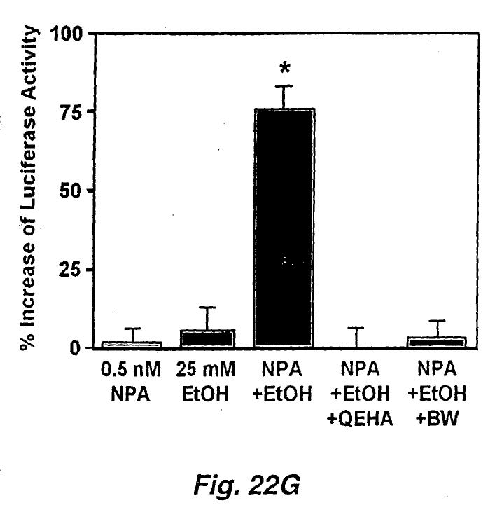

Free βγ dimers of trimeric G proteins bind to the β-adrenergic receptor kinase (PARK) on activation of β-adrenergic receptors (Ruiz-Gomez and Mayor (1997) J. Biol. Chem. 272: 9601-9604). Expression of the carboxyl terminus of βARK1 (βARK1 minigene) binds free βγ dimers and inhibits βγ signaling (Koch et al. (1994) J. Biol. Chem. 269: 6193-6197). PKA Cα translocation induced by NPA (50 nM) or ethanol (100 mM) is markedly inhibited by overexpression of the dominant negative βARK1 minigene (FIG. 22B). Moreover, this scavenger peptide also blocks synergy of translocation (FIG. 22D). Control cells transfected with the adenoviral vector expressing LacZ without the βARK1 mini-gene did not show inhibition of translocation (FIGS. 22B, 22C and 22D). These data suggest that NPA and ethanol-induced release of βγ dimers are required for PKA Cα translocation.

The βARK1 minigene could affect other proteins because a variety of βγ subunits bind to different amino acid sequences (Hamm (1998) J. Biol. Chem. 273: 669-672). Therefore, we transfected cells with a second dominant negative βγ scavenger peptide, QEHA, which is derived directly from ACII and is the corresponding binding site for βγ subunits on AC II (Chakrabarti et al. (1998) Mol. Pharm. 54: 655-662; Chen et al. (1995) Science, 268: 1166-1169; Weng et al (1996) J. Biol. Chem. 271: 26445-26448). QEHA inhibited translocation of PKA Cα induced by NPA (50 nM) or ethanol (100 mM) (FIG. 22C) and synergy of translocation (FIG. 22D). QEHA also prevented NPA- and ethanol-induced increases in CRE-mediated gene expression (FIG. 22F) and synergy produced by subthreshold concentrations of NPA and ethanol (FIG. 22G). QEHA did not inhibit forskolin-induced PKA Cα translocation (FIG. 22C), presumably because forskolin does not require βγ subunits to activate AC isozymes and (Yan et al. (1998) Mol. Pharm. 53: 182-187). Overexpression of the inactive peptide SKEE from an analogous region of ACIII (Chen et al. (1995) Science, 268: 1166-1169) did not affect NPA or ethanol-induced PKA Cα translocation (data not shown). Taken together, these data suggest that release of βγ dimers from Gi/o is required for NPA or ethanol-induced translocation of PKA Cα to the nucleus, subsequent activation of CRE-mediated gene expression, and synergy.

NPA and Ethanol-Induced Synergy: Role of Adenosine Receptors

We have shown that ethanol-induced increases in cAMP require A2 activation (Sapru et al. (1994) J. Pharmacol. Exp. Ther. 271: 542-548). BW A1434U, an adenosine receptor antagonist, blocks PKA Cα translocation (FIG. 22E) and CRE-mediated gene expression (FIG. 22F) induced by 100 mM ethanol. In contrast, Cα translocation (FIG. 4E) and CRE-mediated gene expression (FIG. 22F) induced by 50 nM NPA is not affected by BW A1434U.

BW A1434U blocks NPA and ethanol-induced synergy of Cα translocation (FIG. 22E) and CRE-mediated gene expression (FIG. 22G). Inhibition by BW A1434U is most likely due to A2 receptors, since we find no evidence for A1 receptors in NG108-15 cells (see Discussion). A schematic model illustrating the interaction of D2 and ethanol/A2 receptor pathways and the central role of βγ dimers in mediating ethanol-induced PKA Cα translocation and gene expression is presented in FIG. 25.

NPA and Ethanol Cause PKA Cα Translocation and Exhibit Synergy in Primary Hippocampal Neurons.

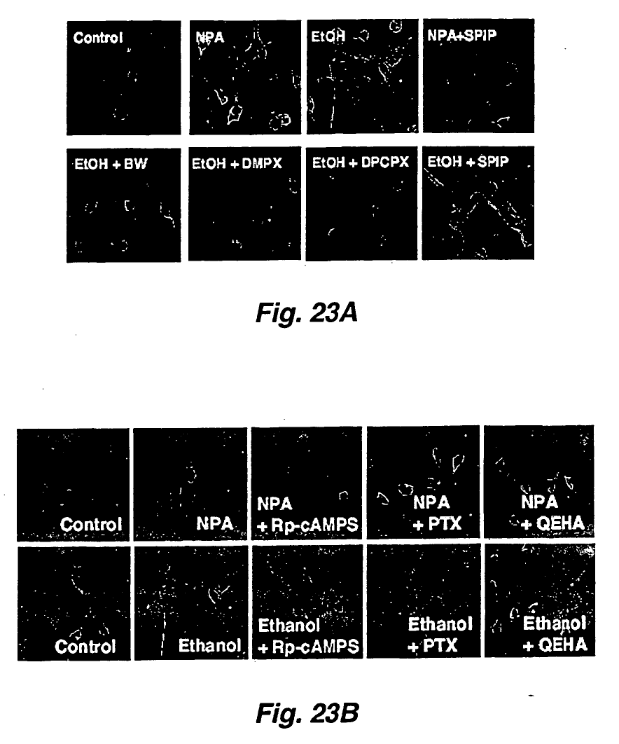

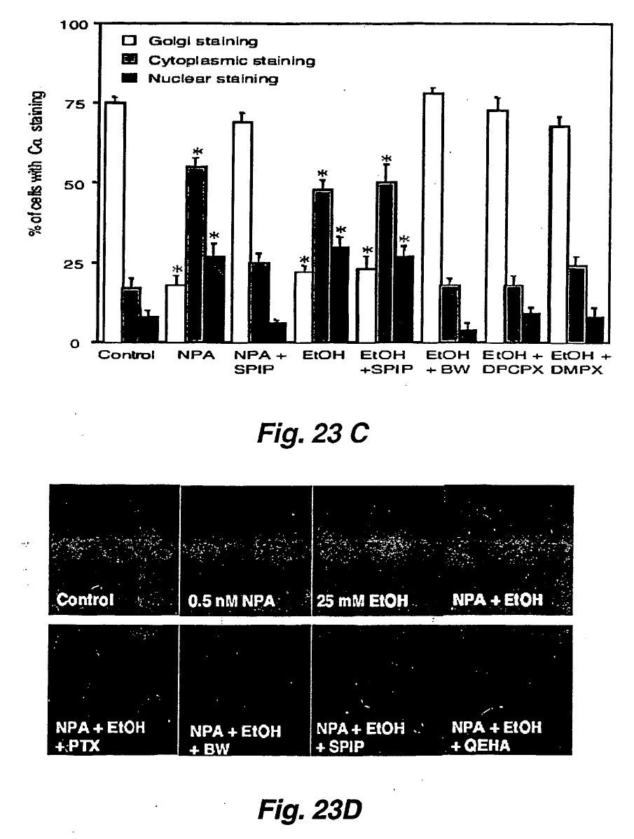

Primary hippocampal neurons in culture express dopamine and adenosine receptors coupled to second messenger signaling pathways (Goldsmith and Joyce (1994) Hippocampus 4: 354-373; O'Kane and Stone (1998) Eur. J. Pharmacology 362: 17-25). We used these cells to extend our findings to non-transformed neurons. FIG. 23A shows that NPA (50 nM) or ethanol (100 mM) induce PKA Cα translocation to the cytoplasm, neurites and nucleus. Spiperone blocks NPA-induced Cα translocation, but has no effect on ethanol-induced translocation. The adenosine receptor antagonist BW A1434U blocks ethanol-induced translocation of Cα (FIG. 23A). Unlike NG108-15/D2 cells, hippocampal neurons express A1 and A2 receptors (O'Kane and Stone (1998) Eur. J. Pharmacology 362: 17-25). Specific blockade of either A1 with DPCPX or A2 with DMPX prevents ethanol-induced Cα translocation (FIG. 23A); they do not affect NPA-induced translocation. Rp-cAMPS, PTX and overexpression of QEHA peptide prevent ethanol or NPA-induced PKA Cα translocation (FIG. 23B). FIG. 5C shows quantitation of this data. BW A1434U, DPCPX, DMPX, spiperone, QEHA, PTX, or Rp-cAMPS alone have no effect on PKA Cα localization (data not shown). Preincubation with 1 μM TTX for 2 hours was without effect (data not shown), indicating that presynaptic neurotransmitter release was not involved.

Synergy of PKA Cα translocation occurs in primary hippocampal neurons at subthreshold doses of NPA (0.5 nM) and ethanol (25 mM) added together (FIG. 23D). Spiperone, PTX, BW A1434U, and QEHA all prevent synergy (FIG. 23D). These data suggest that non-transformed neurons also exhibit similar D2- and ethanol-induced changes in PKA translocation and gene expression via free βγ dimers from Gi/o.

Overexpression of βARK1. a Dominant Negative βγ Scavenger Peptide, Decreases Ethanol Consumption

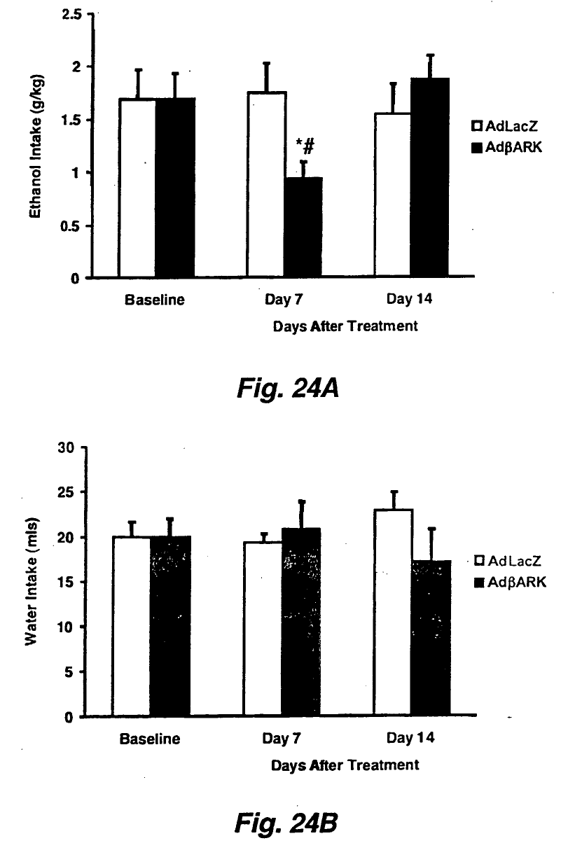

NAc neurons express D2 and A2 in the same cells (Fink et al. (1992) Molecular Brain Research 14: 186-195) and would be expected to release βγ dimers in response to ethanol. The potential role of βγ dimers in ethanol drinking was studied in a continuous voluntary two-bottle choice paradigm in rats. Ethanol consumption was examined for three weeks following bilateral microinjection of the Ad5βARK1 and control Ad5lacZ viral vectors into the medial NAc. Ethanol intake progressively decreased in rats receiving Ad5βARK, with a maximal reduction at 7 days and recovery 14 days after injection (FIG. 24A). Statistical analyses revealed a significant decrease in ethanol intake (g/kg) on Day 7 as compared to Baseline and Day 14 in experimental subjects, and as compared to control Ad5lacZ-injected subjects on Day 7 [ANOVA, Treatment (Ad5lacZ vs. Ad5βARK) by Day (Baseline, Day 7, and Day 14) interaction: F(2,24)=5.39, p<0.015. Ad5βARK Day 7 vs. Baseline, p<0.008; Ad5βARK Day 7 vs. Day 14, p<0.002; Ad5lacZ Day 7 vs. Ad5βARK Day 7, p<004.]. Water intake by Ad5βARK 1- and Ad5lacZ-treated rats was not affected (FIG. 24B). The small amount of microgliosis expected by injections did not differ in control and treated rats (data not shown).

### Discussion.

In this example we show that a D2 agonist activates PKA signaling from increases in cAMP to CRE-mediated gene expression in a neural cell line. The mechanism involves D2 coupling to Gi/o, release of βγ dimers, activation of AC II and/or IV, translocation of PKA Cα to the nucleus and subsequent PKA-dependent increases in gene expression. Ethanol also activates PKA Cα translocation and gene expression in these cells. We find a remarkable synergy between D2 and ethanol-induced activation. Sub-threshold concentrations of NPA or ethanol, which have no effect alone, when added together induce translocation of PKA and activation of CRE-mediated gene expression. Synergy appears to be due to βγ stimulation of AC II and/or IV concomitant with ethanol/A2 activation of AC. The functional significance of PKA translocation induced by subthreshold levels of NPA and ethanol is suggested directly by synergistic increases in CRE mediated gene expression and indirectly by decreases in ethanol consumption caused by expression of a dominant negative βγ scavenger peptide in the NAc.

There is extensive evidence that dopaminergic mechanisms contribute to ethanol consumption Phillips et al. (1998) Nature Neurosc. 1: 610-615). In addition, the PKA pathway has also been implicated in ethanol drinking behavior. Mice heterozygous for Gαs with reduced adenylyl cyclase activity and mice expressing a dominant negative inhibitor of a PKA regulatory subunit with reduced PKA activity (Abel et al. (1997) Cell 88: 615-626) both exhibit increased sensitivity to ethanol sedation and reduced ethanol preference and consumption (Wand et al. (2001) J. Neurosci. 21: 5297-5303). However, mice lacking the RIIβ regulatory subunit of PKA, with reduced cAMP-dependent PKA activity, exhibit resistance to ethanol sedation and increased ethanol preference and consumption (Thiele et al. (2000) J. Neurosci. 20: RC75 (1-6)). It appears, therefore, that relationship between PKA activity and drinking behavior is complex and not well understood. Here we report that overexpression of dominant negative βγ scavenger peptides block D2 receptor and ethanol-induced PKA Cα translocation, gene expression and synergy, probably by preventing βγ activation of AC activity. We also report that expression of the βARK1 minigene βγ scavenger peptide in rat medial NAc reduces voluntary ethanol intake in a two-bottle choice assay, while water consumption is unaffected. Our data provide the first evidence to suggest that 07 dimers in a specific brain reward location are required to sustain voluntary ethanol consumption. Moreover, this finding depends on direct intervention not affected by developmental compensatory changes, which limit interpretation of data derived from genetically engineered mice.

FIG. 25 illustrates a proposed interrelationship between D2 (blue arrows) and ethanol (red arrows) activation of PKA signaling. D2 and A2 receptors activate AC via βγ and Gαs, respectively. Ethanol also promotes the release of 67 dimers from Gi/o while activating the A2 Gs-coupled pathway. Increased cAMP levels lead to release of the catalytic subunit(s) from the regulatory subunit(s) of the PKA holoenzyme and translocation of Cα to the nucleus where it phosphorylates CREB and initiates gene transcription from CRE.

Because D2 and ethanol separately activate the same PKA signaling pathway, we determined whether NPA and ethanol might act synergistically at subthreshold concentrations of each agent. Indeed, we find that NPA-induced PKA Cα translocation and CRE-mediated gene 1 transcription is synergistic with that produced by ethanol. D2-dependent release of free βγ subunits from Gi/o appears to be required for synergy. This is suggested by our observations that the D2 receptor antagonist, spiperone, or the Gi/o inhibitor PTX each blocks synergy of PKA Cα translocation and gene expression, and that overexpression of dominant negative peptides that prevent βγ activation of AC II and IV also blocks synergy of PKA Cα translocation and gene expression.

We have previously reported that ethanol increases extracellular adenosine and activates adenosine receptors in NG108-15 cells (Nagy et al. (1990) J. Biol. Chem., 265: 1946-1951). This increase in extracellular adenosine is required for ethanol-induced increases in cAMP production (Gordon et al. (1986) Proc. Natl. Acad. Sci., USA. 83: 2105-2108; Sapru et al. (1994) J. Pharmacol. Exp. Ther. 271: 542-548), ethanol-induced PKA Cα translocation and CRE-mediated gene expression. A2 is required for synergy of NPA and ethanol-induced PKA signaling in NG108-15/D2 cells. We have never found evidence for A1 receptors in any of our NG108-15 cell lines either by binding of radiolabeled A1 receptor antagonists or changes in AC activity in the presence of A1 receptor agonists (Gordon and Diamond, unpublished observations). Primary hippocampal neurons, however, express functional A1 receptors (Swanson et al. (1995) J. Comp. Neurol. 363: 517-531) and it is likely ethanol-induced increases in extracellular adenosine in hippocampal neurons activate A1 as well as A2 receptors. Indeed, both receptors appear to contribute to ethanol-induced PKA Cα translocation because A1 and A2 antagonists block this enzyme. A1 receptors probably increase cAMP levels via βγ release since they have been shown to couple to Gi/o (Wise et al. (1999) Biochemistry, 38: 2272-2278) just like D2 receptors. Taken together, our data suggest that adenosine and adenosine receptors play a critical role in synergy of PKA signaling induced by subthreshold concentrations of dopamine agonists and ethanol. This is consistent with other studies in which A1 and A2 mediate ethanol induced behavioral responses. A1 agonists and antagonists increase and decrease acute incoordinating effects of ethanol, respectively (Dar (2001) Brain Res. Bull. 55: 513-520). Moreover, ethanol-induced release of β-endorphins from hypothalamic neurons is associated with increases in cAMP, apparently as a result of A2 activation (Boyadjieva and Sarkar 1999) Brain Research 824:112-118).

The results in this example raise the possibility that synergy produced by subthreshold concentrations of dopamine and ethanol may be a pathophysiologic mechanism that not only alters intracellular localization of PKA Cα, but also cAMP-dependent gene expression. Indeed, Thibault et al. (2000) Mol. Pharm. 58: 1593-1600, have demonstrated a subfamily of ethanol-regulated genes that are also regulated by cAMP. Concurrent expression of D2 and A2 is a characteristic of medium spiny neurons in the NAc (Fink et al. (1992) Molecular Brain Research 14: 186-195), a brain region implicated in reward and reinforcement of excessive drinking. Our results suggest that such neurons which express D2 and A2 on the same cells will show a synergistic response at subthreshold concentrations of NPA and ethanol. Our data also suggest that βγ dimers released in NAc neurons might play a role in sustaining drinking behavior. This hypothesis is supported by our experiments designed to determine whether release of βγ dimers is required for continual ethanol consumption. In a two-bottle choice paradigm where rats are allowed continuous access to ethanol, we found that bilateral microinjection of the Ad5βARK minigene into the medial NAc decreased voluntary intake of ethanol. Expression of this dominant negative βγ scavenger peptide selectively reduces ethanol intake 7 days after bilateral microinjection. Injection of a control adenovirus vector (Ad5LacZ) did not affect ethanol intake during the same period, indicating that reduction in drinking behavior is likely due to the expression of the βARK1 minigene, rather than non-specific effects of the adenovirus itself. Rats recovered their pre-injection levels of ethanol intake by day 14 after injection, in agreement with the time course for results obtained with other adenoviral vectors (Thanos et al. (2001) J. Neurochem. 78: 1094-1103). Water intake was not reduced by injection of the Ad5βARK minigene, strengthening the interpretation that the decreases observed are specific to ethanol intake and do not reflect a general attenuation of consummatory behaviors. These results strongly suggest that expression of a βγ scavenger peptide in the medial NAc reduces voluntary consumption of ethanol. The adenoviral vectors we used are known to infect both glial and neuronal cell populations, but the exact mechanism whereby the βARK1 minigene inhibits ethanol consumption is not known and requires further study. However, we found in cell culture that both D2 and ethanol (via A2 and Gi) trigger the PKA-signaling pathway individually and synergistically. Because activation requires free βγ subunits, we propose that the decrease in ethanol consumption observed after inhibiting βγ function in vivo may reflect a disruption of dopamine- and ethanol-induced PKA activation.

The model we present in FIG. 25 suggests that neurons expressing both D2 and A2 receptors are uniquely sensitive to low concentrations of ethanol in the presence of endogenous dopamine. Synergy between dopamine and ethanol requires activation of D2 with release of βγ subunits concomitantly with activation of A2 by adenosine. The NAc is one of the few areas of the brain that expresses both receptors on the same cell (Fink et al. (1992) Molecular Brain Research 14: 186-195). It is tempting to speculate, therefore, that the simultaneous expression of D2 and A2 receptors in neurons in the NAc may account for the central role of this brain region in regulating ethanol intake. It is possible that drugs that target βγ and/or adenosine and dopamine receptors might prevent, attenuate or reverse excessive drinking. Studies are underway to test this possibility.

### Experimental Procedures

All reagents were purchased from Sigma (St. Louis, Mo.) except where indicated. Ham's F-12 medium was purchased from Gibco (Grand Island, N.Y.), NPA and spiperone from Sigma/RBI (Natick, Mass.), Rp-cAMPS from BioLog (La Jolla, Calif.). p-CRE-luciferase was from Stratagene (La Jolla, Calif.) and pCMV-β-galactosidase from QIAGEN (Hilden, Germany).

Cell Culture

NG108-15/D2 cell culture was carried out as described (Gordon et al. (2001) Mol. Pharm. 58: 153-160). Briefly, NG108-15 cells stably expressing the rat D2L receptor (Asai et al. (1998). Alcohol Clin. Exp. Res. 22: 163-166) were grown on single well slides in defined media for 3 days (Dohrman et al. (1996) Proc. Natl. Acad. Sci. USA 93: 10217-10221). 48 hr after plating, media were replaced daily with defined media. On day 4, the cells were treated as described in the Figure Legends and fixed as described below (Gordon et al. (1997) Mol. Pharm. 52: 554-559). Media were replaced daily.

Rat Hippocampal Primary Culture

Hippocampi of newborn (postnatal day 0) Sprague Dawley rat pups were removed and the dentate gyri and fimbria dissected. Cells were prepared and plated at 5-7×10⁴ cells/slide as described previously (Lissin et al. (1999) J. Neurosci. 19: 1263-1272). One-half of the growth medium was changed 1 day after plating and weekly thereafter. On day 10, cells were treated as described in the Figure Legends and fixed as described below.

Cell Culture Immunocytochemistry

NG108-15/D2 cells were fixed with cold methanol (Dohrman et al. (1996) Proc. Natl. Acad. Sci. USA 93: 10217-10221). Primary hippocampal neurons were fixed in 4% formaldehyde containing 120 mM sucrose for 10 min. All preparations were rinsed with phosphate-buffered saline (PBS) and pre-incubated with blocking buffer followed by incubation with primary antibodies specific for PKA Cα (a gift from Dr. Susan Taylor, University of California, San Diego, Calif.), AC type II and type IV (Santa Cruz Biotechnology, Inc., Santa Cruz, Calif.), and neuron-specific nuclear protein (NeuN) (Chemicon International, Inc., Temecula, Calif.), as described (Dohrman et al. (1996) Proc. Natl. Acad. Sci. USA 93: 10217-10221). The cells were then washed, incubated with FITC or TR-conjugated anti-rabbit or anti-mouse secondary antibody (Cappel, Aurora, Ohio) (diluted 1:1000), washed again and coverslipped as described (Dohrman et al, supra.). No staining was observed when primary antibodies were pre-incubated with excess of peptide antigen (Dohrman et al., supra.). Similarly, no staining was observed for any of the primary antibodies in the absence of FITC or TR-conjugated anti-rabbit or anti-mouse secondary antibody. Specificity of the antibodies was further confirmed by western blot analysis of NG108-15/D2 cells. No bands were observed when the primary antibodies were pre-absorbed with Cα protein (data not shown).

Confocal Microscopy

Cells were imaged using a Bio-Rad 1024 scanning laser confocal microscope equipped with a krypton-argon laser attached to a Nikon Optiphot microscope, as described (Gordon et al. (2001) Mol. Pharm. 58: 153-160). Collected data were processed using NIH Image and Adobe Photoshop software. Images are shown as a single plane near the center of the cell. Quantitative data were obtained by two independent observers who were blind to the experimental conditions.

Cell Fractionation

NG 108-15/D2 cells in 100 mm dishes (2×106 cells/dish) were exposed to reagents for 10 min, washed with cold PBS and lysed on ice in 0.5 ml lysis buffer containing 50 mM Tris-HCL, pH 7.4, 2.5 mM MgCL2, 1 mM EDTA, 1 mM DTT, 10% glycerol and protease inhibitors (0.1 mM phenylmethyl sulfonyl fluoride, 20 μg/ml soybean trypsin inhibitor, 25 μg/ml aprotinin, 25 μg/ml leupeptin, and 1 mM sodium orthovanadate). Cells were then homogenized with 10 passes through a 1 ml syringe with a 26-gauge needle and centrifuged at 3000 rpm for 5 min at 40 C. The nuclear pellet was further washed in lysis buffer. The supernatant was centrifuged for 20 min at 150,000 g to separate the membrane fraction (pellet) from the cytosol fraction (supernatant).

Western Blotting

Proteins were subjected to SDS/PAGE and then transferred to polyvinylidene difluoride membranes. Blots were probed using antibodies against PKA C□(1:1000) or AC II or IV (Santa Cruz). The secondary antibody was horseradish peroxidase-linked goat anti-rabbit (1:1000) (NEN BioLabs, Beverly, Mass.). Proteins were detected using LumiGLO chemiluminescence substrate (NEN BioLabs) followed by exposure to Kodak Biomax film.

Construction and Production of Recombinant Adenoviral Vectors and Transfection of Cells

Recombinant Ad5QEHA, Ad5SKEE, Ad5 βARK1, Ad5LacZ and Ad5GFP vectors were created by replacing the E1A gene of the adenovirus with a peptide (QEHA) encoding residues 956 to 982 of AC2, a control peptide (SKEE) corresponding to the cognate region of AC3, the carboxyl terminus (Gly495 to Leu689) of bovine βARK1, LacZ or the GFP gene, respectively. All are under control of a CMV promoter. To make the recombinant adenoviruses, the cosmids (Stratagene, La Jolla, Calif.) containing recombinant adenovirus DNA were transfected into 50%-80% confluent HEK 293 cells. Two weeks later, cells were harvested by brisk pipeting, resuspended in PBS and lysed by three cycles of rapid freezing in an ethanol/dry ice bath and thawing at 37 ¢^{a}C. The lysate was then centrifuged at 600× g to remove debris and the supernatant saved. For large-scale production, HEK293 cells were reinfected with the virus stock and harvested 3 days later. Viruses were purified by CsCl banding and dialyzed three times against PBS. To determine viral titers, HEK293 cells were seeded in six-well plates. Twenty-four hours after plating, they were infected with serial dilutions of the virus stocks. Two hours after infection, the medium was removed and cells were overlaid with a medium containing 2% fetal bovine serum and 1% agarose. Two weeks later, the number of virus plaques was recorded. To transfect NG108-15/D2 cells and primary neurons from hippocampus, culture media were replaced with media containing Ad5QEHA, Ad5SKEE, Ad5βARK1 or Ad5GFP at a MOI (multiplicity of infection) of 100 and the cells were cultured 20-24 hr prior to experiments.

CRE-Luciferase Reporter Assay

NG108-15/D2 cells were plated at 1×10⁶ cells/100 mm plate, grown in defined medium for 3 days, and then transfected with 10 μg of CRE-luciferase construct using the LipofectAMINE reagent (GIBCOL) as recommended by the manufacturer. Cells were cultured for 24 h post transfection before being seeded into 12-well plates. Following a further 2 day culture, cells were treated for 10 min with or without 50 nM NPA or 100 mM ethanol; with or without 0.5 nM NPA or 25 mM ethanol alone or in combination after preincubation with or without 10 μM BW A1434U, 10 μM spiperone or 20 μM Rp-cAMPS for 30 min, 50 ng/ml PTX or QEHA overnight. Cells were then washed with PBS and cultured for a total of 5 h. Cell extracts were prepared and luciferase activity measured with a luciferase assay system (Promega, Madison, Wis.), using a Rosys Anthos Lucy2 microplate luminometer (Anthos Labtec Instruments, Austria). Results are expressed as percent increase over control. Data represent three or more separate experiments from triplet wells. Luciferase activities were normalized for transfection efficiency determined in cells transfected in parallel with pCMVβgalactosidase. β-galactosidase activity was measured using a kit from Strategene (La Jolla, Calif.).

Two Bottle Choice Paradigm

Voluntary ethanol intake was measured in individually-housed Male Long Evans rats, 250-300 g at time of arrival, using a standard continuous two-bottle choice paradigm. Briefly, following a 4-day forced exposure to 10% ethanol as die only liquid source to overcome their innate neophobia, rats were allowed access to 2 50-ml glass bottles in their home cages, one containing 10% ethanol in tap water and the other tap water. Rats were weighed and their fluid intake was measured every day at the same time. Forty-five days later, rats underwent surgery to receive permanent bilateral cannulae in the NAc, as described below. After surgery, rats continued 12-15 days with the two bottle choice paradigm until their drinking behavior varied < 10% from their pre-surgery baseline. At this time, rats received microinjections of either Ad5□ARK1 or Ad5LacZ (β-galactosidase control). Fluid intake was recorded for an additional 20 days. This procedure and all others in this report that were performed with rats were approved by the Gallo Center Institutional Animal Care and Use Committee.

Stereotaxic Surgery. Cannulae Implantation and Viral Vector Microinjection

Rats were anesthetized with isoflurane (Aerrane, Baxter, Deerfield, Ill.) and implanted with bilateral cannulae (26 gauge; Plastics One, Inc., Roanoke, Va.) aimed to the NAc: +1.7 mm anteroposterior (AP), ±1.0 mm mediolateral (ML), −6.4 mm dorsoventral (DV) (relative to bregma; Paxinos and Watson, 1998). Twelve to fifteen days post-surgery, animals were anesthetized with isoflurane and received 2 μl (10¹² pfu/ml)/side of Ad5βARK1 or Ad5LacZ viral vector constructs over 10 min through 33 gauge injection cannulae (Plastics One, Inc.) via an infusion pump (model II, Harvard Apparatus, Inc., Holliston, Mass.). Infusion cannulae were left in place an additional min to ensure diffusion of the drug.

Histologic Verification of Cannula Placement

Twenty days post-injection, rats were sacrificed with isoflurane and perfused with 0.9% saline followed by 4% paraformaldehyde in PBS. Cannula placement was evaluated in thionin-stained 50 μl coronal brain slices using the atlas of Paxinos and Watson (1998) The rat brain in stereotaxic coordinates. 4th Ed., Academic Press, San Diego. Only those rats with bilateral placements in the NAc were included in the groups for statistical analysis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of mitigating one or more symptoms associated with chronic consumption of a substance of abuse by a mammal, said method comprising:
administering to said mammal an effective amount of an adenosine receptor antagonist; and
an effective amount of a dopamine receptor antagonist;
wherein the effective amount of the adenosine receptor antagonist is lower than the effective amount of an adenosine receptor antagonist administered without said dopamine receptor antagonist.

2. The method of claim 1, wherein the effective amount of the dopamine receptor antagonist is lower than the effective amount of a dopamine receptor antagonist administered without said adenosine receptor antagonist.

3. The method of claim 1, wherein the combined dosage of dopamine receptor antagonist and adenosine receptor antagonist is just sufficient to produce minimum activation of PKA in response to consumption of a substance of abuse.

4. The method of claim 1, wherein the dopamine receptor antagonist is administered at a standard therapeutic dosage.

5. The method of claim 1, wherein the dopamine receptor antagonist is administered at about a threshold dosage.

6. The method of claim 1, wherein the dopamine receptor antagonist is administered at a sub-threshold dosage.

7. The method of claim 1, wherein the adenosine receptor antagonist is administered at a standard therapeutic dosage.

8. The method of claim 1, wherein the adenosine receptor antagonist is administered at about a threshold dosage.

9. The method of claim 1, wherein the adenosine receptor antagonist is administered at a sub-threshold dosage.

10. The method of claim 1, wherein said substance of abuse is selected from the group consisting of an opioid, a barbiturate, a cannabinoid, cocaine, an amphetamine, alcohol, and nicotine.

11. The method of claim 1, wherein said dopamine receptor antagonist is selected from the group consisting of butaclamol, chlorpromazine, domperidone, fluphenazine, haloperidol, heteroaryl piperidines, metoclopramide, olanzapine, perospirone hydrochloride hydrate, phenothiazine, pimozide, quetiapine, risperidone, sertindole, sulpiride, ziprasidone, and zotepine.

12. The method of claim 1, wherein the effective dosage of the dopamine receptor antagonist is low enough to avoid causing an adverse symptom characteristically produced by administration of a dopamine receptor antagonist.

13. The method of claim 12, where said adverse symptom is selected from the group consisting of tardive dyskensia, dystonia, and neuroendocrine (hormonal) disturbances.

14. The method of claim 1, wherein said adenosine receptor antagonist is selected from the group consisting of PD115,199; ZM 241385, quinazoline, 3-(3-hydroxyphenyl)-5H-thiazolo[2,3b]-quinazoline, 1,3-diethyl-8-phenylxanthine, and other substituted phenylxanthines.

15. The method of claim 1, wherein the effective dosage of the adenosine receptor antagonist is low enough to avoid causing an adverse symptom characteristically produced by administration of an adenosine receptor antagonist.

16. The method of claim 15, where said adverse symptom is selected from the group consisting of sleep disorders, elevated heart rate, and arrhythmia.

17. The method of claim 1, wherein the dopamine receptor antagonist and the adenosine receptor antagonist are administered sequentially.

18. The method of claim 1, wherein the dopamine receptor antagonist and the adenosine receptor antagonist are administered simultaneously.

19. The method of claim 1, wherein the dopamine receptor antagonist and the adenosine receptor antagonist are administered in a single unit dosage formulation.

20. The method of claim 1, wherein said symptom is a chronic consumptive behavior.

21. A composition comprising an effective amount of an adenosine receptor antagonist; and an effective amount of a dopamine receptor antagonist, wherein the effective amount of the adenosine receptor antagonist is lower than the effective amount of an adenosine receptor antagonist administered without said dopamine receptor antagonist.

22. The composition of claim 21, wherein the effective amount of the dopamine receptor antagonist is lower than the effective amount of a dopamine receptor antagonist administered without said adenosine receptor antagonist.

23. The composition of claim 21, wherein the combined dosage of dopamine receptor antagonist and adenosine receptor antagonist is just sufficient to-inhibit activation of PKA in response to consumption of a substance of abuse.

24. The composition of claim 21, wherein the dopamine receptor antagonist is at a standard therapeutic dosage.

25. The composition of claim 21, wherein the dopamine receptor antagonist is at about a threshold dosage.

26. The composition of claim 21, wherein the dopamine receptor antagonist is at a sub-threshold dosage.

27. The composition of claim 21, wherein the adenosine receptor antagonist dosage at a standard therapeutic dosage.

28. The composition of claim 21, wherein the adenosine receptor antagonist is at about a threshold dosage.

29. The composition of claim 21, wherein the adenosine receptor antagonist is at a sub-threshold dosage.

30. The composition of claim 21, wherein said dopamine receptor antagonist is selected from the group consisting of butaclamol, chlorpromazine, domperidone, fluphenazine, haloperidol, heteroaryl piperidines, metoclopramide, olanzapine, perospirone hydrochloride hydrate, phenothiazine, pimozide, quetiapine, risperidone, sertindole, sulpiride, ziprasidone, and zotepine.

31. The composition of claim 21, wherein the effective dosage of the dopamine receptor antagonist is low enough to avoid causing an adverse symptom characteristically produced by administration of a dopamine receptor antagonist.

32. The composition of claim 31, where said adverse symptom is selected from the group consisting of tardive dyskensia, dystonia, and neuroendocrine (hormonal) disturbances.

33. The composition of claim 21, wherein said adenosine receptor antagonist is selected from the group consisting of f PD115,199; ZM 241385, quinazoline, 3-(3-hydroxyphenyl)-5H-thiazolo[2,3b]-quinazoline, 1,3-diethyl-8-phenylxanthine, and other substituted phenylxanthines.

34. The composition of claim 21, wherein the effective dosage of the adenosine receptor antagonist is low enough to avoid causing an adverse symptom characteristically produced by administration of an adenosine receptor antagonist.

35. The composition of claim 21, wherein the dopamine receptor antagonist and the adenosine receptor antagonist in a single unit dosage formulation.

36. A method of mitigating one or more symptoms associated withdrawal associated with cessation of consumption of a substance of abuse by a mammal, said method comprising:
administering to said mammal an effective amount of an adenosine receptor agonist; and
an effective amount of a dopamine receptor agonist;
wherein the effective amount of the adenosine receptor agonist is lower than the effective amount of an adenosine receptor agonist administered without said dopamine receptor agonist.

37. The method of claim 36, wherein the combined amount of dopamine receptor agonist and adenosine receptor agonist is sufficient to maintain changes in gene expression associated with chronic consumption of a substance of abuse.

38. The method of claim 36, wherein the combined amount of dopamine receptor agonist and adenosine receptor agonist is just sufficient to activate a PKA pathway.

39. The method of claim 36, wherein the combined amount of dopamine receptor agonist and adenosine receptor agonist is sufficient to reduce a symptom of withdrawal from a substance of abuse.

40. The method of claim 36, wherein the effective amount of the dopamine receptor agonist is lower than the effective amount of a dopamine receptor agonist administered without said adenosine receptor agonist.

41. The method of claim 36, wherein the dopamine receptor agonist is administered at a standard therapeutic dosage.

42. The method of claim 36, wherein the dopamine receptor agonist is administered at about a threshold dosage.

43. The method of claim 36, wherein the dopamine receptor agonist is administered at a sub-threshold dosage.

44. The method of claim 36 wherein the adenosine receptor agonist is administered at a standard therapeutic dosage.

45. The method of claim 36, wherein the adenosine receptor agonist is administered at about a threshold dosage.

46. The method of claim 36, wherein the adenosine receptor agonist is administered at a sub-threshold dosage.

47. The method of claim 36, wherein said substance of abuse is selected from the group consisting of an opioid, a barbiturate, cocaine, an amphetamine, a cannabinoid, alcohol, and nicotine.

48. The method of claim 36, wherein said dopamine receptor agonist is selected from the group consisting of Bromocriptine, CY 208-243, SKF 83959, ABT-431, SKF 38393, SKF 81297, LY 171555, R(−)-10, and 11-dihydroxy-N-n-propylnorapomorphine (NPA).

49. The method of claim 36, wherein the effective dosage of the dopamine receptor agonist is low enough to avoid causing an adverse symptom characteristically produced by administration of a dopamine receptor agonist.

50. The method of claim 49, where said adverse symptom is orthostatic hypotension.

51. The method of claim 36, wherein said adenosine receptor agonist is selected from the group consisting of: CGS21680 (Ciba-Geigy), 2-phenylaminoadenosine (CV1808), N,6-cyclohexyl-adenosine, and N,6-cyclopentyladenosine.

52. The method of claim 36, wherein the effective dosage of the adenosine receptor agonist is low enough to avoid causing an adverse symptom characteristically produced by administration of an adenosine receptor agonist.

53. The method of claim 52, where said adverse symptom is selected from the group consisting of drowsiness, inability to concentrate, uncoordination, and sedation,

54. The method of claim 36, wherein the dopamine receptor agonist and the adenosine receptor agonist are administered sequentially.

55. The method of claim 36, wherein the dopamine receptor agonist and the adenosine receptor agonist are administered simultaneously.

56. The method of claim 36, wherein the dopamine receptor agonist and the adenosine receptor agonist are administered in a single unit dosage formulation.

57. The method of claim 36, wherein said symptom is selected from the group consisting of craving for a substance of abuse, delerium tremens, irritability, frustration, anger or anxiety, difficulty in concentrating, restlessness, increased appetite, insomnia, depression, seizures, and relapse.

58. A composition for mitigating a symptom of withdrawal from a drug of abuse, said composition comprising an effective amount of an adenosine receptor agonist; and an effective amount of a dopamine receptor agonist, wherein the effective amount of the adenosine receptor agonist is lower than the effective amount of an adenosine receptor agonist administered without said dopamine receptor agonist.

59. The composition of claim 58, wherein the effective amount of the dopamine receptor agonist is lower than the effective amount of a dopamine receptor agonist administered without said adenosine receptor agonist.

60. The composition of claim 58, wherein the combined amount of dopamine receptor agonist and adenosine receptor agonist is sufficient to maintain changes in gene expression associated with chronic consumption of a substance of abuse.

61. The composition of claim 58, wherein the combined amount of dopamine receptor agonist and adenosine receptor agonist is just sufficient to activate a PKA pathway.

62. The composition of claim 58, wherein the combined amount of dopamine receptor agonist and adenosine receptor agonist is sufficient to reduce a symptom of withdrawal from a substance of abuse.

63. The composition of claim 58, wherein the dopamine receptor agonist is in a dosage range that is a standard therapeutic dosage.

64. The composition of claim 58, wherein the dopamine receptor agonist is at about a threshold dosage.

65. The composition of claim 58, wherein the dopamine receptor agonist is a sub-threshold dosage.

66. The composition of claim 58, wherein the adenosine receptor agonist is a dosage range that is a standard therapeutic dosage.

67. The composition of claim 58, wherein the adenosine receptor agonist is at about a threshold dosage.

68. The composition of claim 58, wherein the adenosine receptor agonist is a sub-threshold dosage.

69. The composition of claim 58, wherein said substance of abuse is selected from the group consisting of an opioid, a barbiturate, a cannabinoid, cocaine, an amphetamine, alcohol, and nicotine.

70. The composition of claim 58, wherein said dopamine receptor agonist is selected from the group consisting of Bromocriptine, CY 208-243, SKF 83959, ABT-431, SKF 38393, SKF 81297, LY 171555, R(−)-10, and 11-dihydroxy-N-n-propylnorapomorphine (NPA).

71. The composition of claim 58, wherein the effective dosage of the dopamine receptor agonist is low enough to avoid causing an adverse symptom characteristically produced by administration of a dopamine receptor agonist.

72. The composition of claim 71, where said adverse symptom is orthostatic hypotension.

73. The composition of claim 58, wherein said adenosine receptor agonist is selected from the group consisting of: CGS21680 (Ciba-Geigy), 2-phenylaminoadenosine (CV1808), N,6-cyclohexyl-adenosine, and N,6-cyclopentyladenosine.

74. The composition of claim 58, wherein the effective dosage of the adenosine receptor agonist is low enough to avoid or reduce an adverse symptom characteristically produced by administration of an adenosine receptor agonist.

75. The composition of claim 74, where said adverse symptom is selected from the group consisting of drowsiness, inability to concentrate, uncoordination, and sedation,

76. The composition of claim 58, wherein the dopamine receptor agonist and the adenosine receptor agonist comprise a single unit dosage formulation.

77. The composition of claim 58, wherein said symptom is selected from the group consisting of craving for a substance of abuse, delerium tremens, irritability, frustration, anger or anxiety, difficulty in concentrating, restlessness, increased appetite, insomnia, depression, relapse, and seizure.

78. A method of mitigating one or more symptoms associated with chronic consumption of a substance of abuse by a mammal, said method comprising inhibiting expression or activity of a beta/gamma dimer.

79. The method of claim 78, wherein said substance of abuse is alcohol.

80. The method of claim 78, wherein the symptom is consumptive behavior.

81. The method of claim 78, wherein said inhibiting expression or activity comprises contacting said beta/gamma dimer with a moiety that specifically binds to said beta/gamma dimer.

82. The method of claim 81, wherein said beta/gamma dimer is a beta-1/gamma 2 dimer.

83. The method of claim 81, wherein said moiety is a beta/gamma dimer scavenger peptide.

84. The method of claim 81, wherein said moiety is a beta/gamma dimer specific antibody.

85. The method of claim 81, wherein said moiety is a beta/gamma dimer specific intrabody.

86. A method of mitigating consumptive behavior or craving after withdrawal of a substance of abuse, said method comprising:
administering to a mammal an agent that increases effective adenosine levels or activity of an adenosine receptor in a concentration sufficient to mitigate said consumptive behavior or craving.

87. The method of claim 86, wherein said agent is an agent that inhibits adenosine reuptake.

88. The method of claim 86, wherein said agent is dipyrrimidole.

89. The method of claim 86, wherein said agent is an adenosine receptor agonist.

90. The method of claim 86, wherein said agent is selected from the group consisting of PD115,199; ZM 241385, quinazoline, 3-(3-hydroxyphenyl)-5H-thiazolo[2,3b]-quinazoline, 1,3-diethyl-8-phenylxanthine, and other substituted phenylxanthines.

91. The method of claim 86, wherein said agent is an adenosine deaminase inhibitor or an adenosine kinase inhibitor.

92. The method of claim 86, wherein said agent is selected from the group consisting of erythro-9[2-hydroxyl-3-nonyl]adenine (EHNA), 2′-deoxycoformycin (DCF), 2′-deoxycoformin, 5′amino-5′-deoxyadenosine, 5′-deoxy-5-iodotubercidin, 5′-iodotubericidin, iodotubericidin (16), iodotubericidin, GP515 (17), 4-(N-phenylamino)-5-phenyl-7-(5′-deoxyribofuranosyl)pyrrolo[2,3-d]pyrimidine (GP683).

93. A method of mitigating consumptive behavior or craving during chronic consumption of a substance of abuse, said method comprising:
administering to a mammal engaging in said chronic consumption of a substance of abuse, an adenosine receptor antagonist in a concentration sufficient to mitigate said consumptive behavior or craving.

94. The method of claim 93 wherein said adenosine receptor antagonist is selected from the group consisting of PD115.199; ZM 241385, quinazoline, 3-(3-hydroxyphenyl)-5H-thiazolo[2,3b]-quinazoline, 1,3-diethyl-8-phenylxanthine, and other substituted phenylxanthines.

95. A method of screening for an agent that modulates the effect of a substance of abuse on PKA activation in a mammalian cell, said method comprising:
contacting a mammalian test cell with a test agent; and
detecting the expression or activity of a beta/gamma dimer of said test cell wherein a difference in beta/gamma dimer expression or activity in said test cell as compared to beta/gamma dimer expression or activity in a control cell indicates that said test agent modulates the effect of a substance of abuse on PKA activation.

96. The method of claim 95, wherein said beta/gamma dimer is a beta-1/gamma-2 dimer.

97. The method of claim 95, wherein a decrease in the expression or activity of a beta/gamma dimmer indicates that said test agent decreases the activation of PKA associated with said substance of abuse.

98. The method of claim 95, wherein said substance of abuse is selected from the group consisting of alcohol, an opiate, a barbituate, a cannabinoid, and nicotine.

99. The method of claim 95, wherein said method further comprises contacting said test cell with said substance of abuse.

100. The method of claim 95, wherein an adenosine receptor pathway in said test cell is activated.

101. The method of claim 95, wherein said test cell comprises αS.

102. The method of claim 95, wherein said test cell comprises PKCα.

103. The method of claim 95, wherein said test cell is cultured ex vivo.

104. The method of claim 95, wherein said test agent is administered to a non-human mammal.

105. The method of claim 95, wherein said control cell is a positive control contacted with said test agent at a higher concentration than said test cell.

106. The method of claim 95, wherein said control cell is a negative control contacted with said test agent at a lower concentration than said test cell.

107. The method of claim 106, wherein said control cell is not contacted with said test agent.

108. The method of claim 95, wherein said test cell is a nerve cell.

109. The method of claim 95, wherein the expression of a beta/gamma dimer is detected by detecting a mRNA encoding a polypeptide that comprises a beta/gamma dimer.

110. The method of claim 109, wherein the level of said mRNA is measured by hybridizing said mRNA to a probe that specifically hybridizes to a nucleic acid that encodes a polypeptide that comprises a beta/gamma dimer.

111. The method of claim 110, wherein said hybridizing is according to a method selected from the group consisting of a Northern blot, a Southern blot using DNA derived from the RNA encoding a polypeptide comprising a beta/gamma dimer, an array hybridization, an affinity chromatography, and an in situ hybridization.

112. The method of claim 110, wherein said probe is a member of a plurality of probes that forms an array of probes.

113. The method of claim 109, wherein the level of mRNA encoding a polypeptide that comprises a beta/gamma dimer is measured using a nucleic acid amplification reaction.

114. The method of claim 95, wherein the expression or activity of a beta/gamma dimer is detected by detecting the level of a beta/gamma dimer in said cell.

115. The method of claim 114, wherein said detecting is via a method selected from the group consisting of capillary electrophoresis, a Western blot, mass spectroscopy, ELISA, immunochromatography, and immunohistochernistry.

116. The method of claim 95, wherein the expression or activity of a beta/gamma dimer is detected by detecting activity of a PCA pathway.

117. The method of claim 116, wherein said activity is detected by detecting translocation of a component of a PKA pathway.

118. A method of screening for an agent that decouples dopamine receptor activity from an adenosine receptor pathway, said method comprising:
contacting a test cell comprising a dopamine receptor with a test agent;
detecting the expression or activity of a beta-gamma dimer wherein a decrease in beta/gamma dimer expression or activity in said cell as compared to beta/gamma dimer expression or activity in a control cell indicates that said test agent decouples dopamine receptor activity from an adenosine receptor pathway

119. A method of prescreening for an agent that modulates the effect of a substance of abuse on PKA activation in a mammalian cell, said method comprising:
i) contacting a beta/gamma dimmer or a nucleic acid that encodes a polypeptide comprising a beta/gamma dimmer with a test agent; and
ii) detecting specific binding of said test agent to a beta/gamma dimmer or to a nucleic acid that encodes a polypeptide comprising a beta/gamma dimer wherein specific binding indicates that said agent is a candidate agent modulates the effect of a substance of abuse on PKA activation in a mammalian cell.

120. The method of claim 119, further comprising recording test agents that specifically bind to a beta/gamma dimmer or to a nucleic acid that encodes a polypeptide comprising a beta/gamma dimer in a database of candidate agents that modulates the effect of a substance of abuse on PKA activation in a mammalian cell.

121. The method of claim 119, wherein said test agent is not an antibody.

122. The method of claim 119, wherein said test agent is a beta/gamma scavenger peptide.

123. The method of claim 119, wherein said test agent is not a protein.

124. The method of claim 119, wherein said test agent is not a nucleic acid.

125. The method of claim 119, wherein said test agent is a small organic molecule.

126. The method of claim 119, wherein said detecting comprises detecting specific binding of said test agent to a nucleic acid that encodes a polypeptide comprising a beta/gamma dimer.

127. The method of claim 126, wherein said binding is detected using a method selected from the group consisting of a Northern blot, a Southern blot using DNA derived from the RNA encoding a polypeptide comprising a beta/gamma dimer, an array hybridization, an affinity chromatography, and an in situ hybridization.

128. The method of claim 119, wherein said detecting comprises detecting specific binding of said test agent to a beta/gamma dimer.

129. The method of claim 128, wherein said detecting is via a method selected from the group consisting of capillary electrophoresis, a Western blot, mass spectroscopy, ELISA, immunochromatography, and immunohistochemistry.

130. The method of claim 119, wherein said test agent is contacted directly to the a beta/gamma dimmer or to a nucleic acid that encodes a polypeptide comprising a beta/gamma dimer.

131. The method of claim 119, wherein said test agent is contacted to a mammalian cell.

132. The method of claim 131, wherein said cell is cultured ex vivo.

133. The method of claim 119, wherein said test agent is administered to a non-human mammal.

134. A composition comprising an adenosine receptor antagonist and a dopamine receptor antagonist in a pharmacologically acceptable excipient.

135. The composition of claim 134, wherein the combination of an adenosine receptor antagonist and dopamine receptor antagonist are sufficient to mitigate a symptom associated with chronic consumption of ethanol.

136. The composition of claim 134, wherein said composition comprises a unit dosage formulation.

137. A kit comprising:
a container containing an adenosine receptor antagonist; and
a container containing a dopamine receptor antagonist.

138. The kit of claim 137, wherein said container containing an adenosine receptor antagonist; and said a container containing a dopamine receptor antagonist are the same container.

139. The kit of claim 137, wherein said adenosine receptor antagonist is in a pharmacologically acceptable excipient.

140. The kit of claim 137, wherein said dopamine receptor antagonist is in a pharmacologically acceptable excipient.

141. The kit of claim 137, further comprising instructional materials teaching the use of a combination of an adenosine receptor antagonist and a dopamine receptor antagonist to mitigate a symptom associated with chronic consumption of a substance of abuse.

142. A composition comprising an adenosine receptor agonist and a dopamine receptor agonist in a pharmacologically acceptable excipient.

143. The composition of claim 142, wherein the combination of an adenosine receptor agonist and dopamine receptor agonist are sufficient to mitigate a symptom associated with withdrawal from chronic consumption of ethanol.

144. The composition of claim 142, wherein said composition comprises a unit dosage formulation.

145. A kit comprising:
a container containing an adenosine receptor agonist; and
a container containing a dopamine receptor agonist.

146. The kit of claim 145, wherein said container containing an adenosine receptor agonist; and said a container containing a dopamine receptor agonist are the same container.

147. The kit of claim 145, wherein said adenosine receptor agonist is in a pharmacologically acceptable excipient.

148. The kit of claim 145, wherein said dopamine receptor agonist is in a pharmacologically acceptable excipient.

149. The kit of claim 145, further comprising instructional materials teaching the use of a combination of an adenosine receptor agonist and a dopamine receptor agonist to mitigate a symptom associated withdrawal from chronic consumption of a substance of abuse.
